(19) United States
(12) Patent Application Publication
(10) Pub. No.: US 2024/0393688 A1
(43) Pub. Date: Nov. 28, 2024

(54) RADIATION-SENSITIVE RESIN COMPOSITION AND PATTERN FORMATION METHOD

(71) Applicant: JSR CORPORATION, Tokyo (JP)

(72) Inventors: Ryuichi NEMOTO, Tokyo (JP); Taiichi FURUKAWA, Tokyo (JP); Tsuyoshi FURUKAWA, Tokyo (JP); Hajime INAMI, Tokyo (JP)

(73) Assignee: JSR CORPORATION, Tokyo (JP)

(21) Appl. No.: 18/796,665

(22) Filed: Aug. 7, 2024

Related U.S. Application Data
(63) Continuation-in-part of application No. PCT/JP2023/003312, filed on Feb. 2, 2023.

(30) Foreign Application Priority Data
Feb. 8, 2022 (JP) 2022-017994

(51) Int. Cl. G03F 7/039; G03F 7/004

(52) U.S. Cl. CPC G03F 7/0392; G03F 7/0045

(57) ABSTRACT

A radiation-sensitive resin composition includes: a polymer comprising a structural unit (I) represented by formula (1) and a structural unit different from the structural unit (I); an onium salt represented by formula (i); and a solvent. In formula (1), R^{K1} is a hydrogen atom, a fluorine atom, a methyl group, or a trifluoromethyl group, L¹ is an alkanediyl group having 1 to 5 carbon atoms, and R^{f1} is a fluorinated hydrocarbon group having 2 to 10 carbon atoms and 5 to 7 fluorine atoms. In formula (i), R^{a1} is a substituted or unsubstituted monovalent organic group having 1 to 40 carbon atoms with no fluorine atom or fluorinated hydrocarbon group attached to an atom adjacent to the sulfur atom, and X⁺ is a monovalent onium cation.

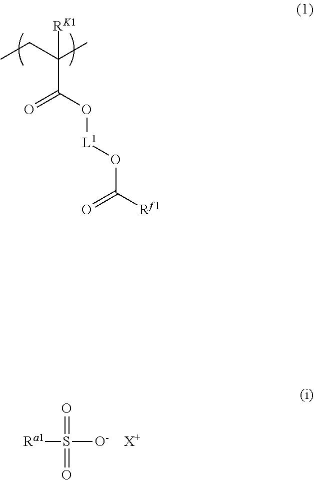

### CROSS-REFERENCE TO RELATED APPLICATIONS

The present application is a continuation-in-part application of International Patent Application No. PCT/JP2023/003312 filed Feb. 2, 2023, which claims priority to Japanese Patent Application No. 2022-017994 filed Feb. 8, 2022. The contents of these applications are incorporated herein by reference in their entirety.

### BACKGROUND OF THE DISCLOSURE

### Technical Field

The present disclosure relates to a radiation-sensitive resin composition and a method for forming a pattern.

### Background Art

A photolithography technology using a resist composition has been used for the fine circuit formation in a semiconductor device. As the representative procedure, for example, a resist pattern is formed on a substrate by generating an acid by irradiating the coating of the resist composition with a radioactive ray through a mask pattern, and then reacting in the presence of the acid as a catalyst to generate the difference of solubility of a resin into an alkaline or organic developer between an exposed part and a non-exposed part.

In the photolithography technique, pattern miniaturization is promoted by using a short-wavelength radiation such as ArF excimer laser or by combining this ArF exposure with an immersion exposure method (liquid immersion lithography).

As regards a resist composition used in the immersion exposure method, addition of a water-repellent compound to the resist composition is being tested for the purpose of improving process efficiency by modifying the surface of the resist film. Proposed is, for example, a technique of adding a water-repellent compound that maintains hydrophobicity in the resist composition but has solubility in an alkaline developer to improve resist performance and defect prevention (see JP-B-6774214).

### SUMMARY

According to an aspect of the present disclosure, a radiation-sensitive resin composition includes: a polymer comprising a structural unit (I) represented by formula (1) and a structural unit different from the structural unit (I); an onium salt represented by formula (i) (hereinafter also referred to as “onium salt (i)”; and a solvent.

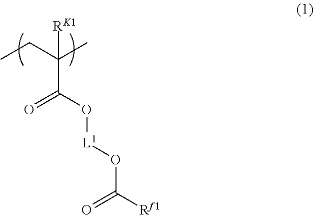

In formula (1), R^{K1} is a hydrogen atom, a fluorine atom, a methyl group, or a trifluoromethyl group, L is an alkanediyl group having 1 to 5 carbon atoms, and R^{f1} is a fluorinated hydrocarbon group having 2 to 10 carbon atoms and 5 to 7 fluorine atoms.

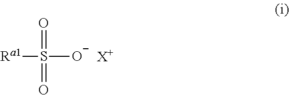

In formula (i), R^{a1} is a substituted or unsubstituted monovalent organic group having 1 to 40 carbon atoms with no fluorine atom or fluorinated hydrocarbon group attached to an atom adjacent to the sulfur atom, and X⁺ is a monovalent onium cation.

According to another aspect of the present disclosure, a method for forming a pattern, includes: directly or indirectly applying the above-described radiation-sensitive resin composition to a substrate to form a resist film; exposing the resist film; and developing the exposed resist film with a developer.

### DESCRIPTION OF THE EMBODIMENTS

As used herein, the words “a” and “an” and the like carry the meaning of “one or more.” When an amount, concentration, or other value or parameter is given as a range, and/or its description includes a list of upper and lower values, this is to be understood as specifically disclosing all integers and fractions within the given range, and all ranges formed from any pair of any upper and lower values, regardless of whether subranges are separately disclosed. Where a range of numerical values is recited herein, unless otherwise stated, the range is intended to include the endpoints thereof, as well as all integers and fractions within the range. As an example, a stated range of 1-10 fully describes and includes the independent subrange 3.4-7.2 as does the following list of values: 1, 4, 6, 10.

In this specification, “organic group” is a group having at least one carbon atom. Unless otherwise indicated, “hydrocarbon group” includes chain hydrocarbon groups, alicyclic hydrocarbon groups and aromatic hydrocarbon groups. The above “hydrocarbon group” includes both saturated and unsaturated hydrocarbon groups. The above “chain hydrocarbon group” refers to a hydrocarbon group that does not contain a cyclic structure and consists only of a chain structure, and includes both linear and branched-chain hydrocarbon groups. The above “alicyclic hydrocarbon group” refers to a hydrocarbon group that contains only an alicyclic structure as a ring structure and no aromatic ring structure, and includes both monocyclic alicyclic hydrocarbon groups and polycyclic alicyclic hydrocarbon groups. However, it is not necessary to consist only of an alicyclic structure, and may include a chain structure in part of the alicyclic structure. The above “aromatic hydrocarbon group” includes hydrocarbon groups that contain an aromatic ring structure as a ring structure. However, it is not necessary that it is composed solely of an aromatic ring structure, and it may contain a chain structure or an alicyclic structure in part.

As the next-generation technology following ArF exposure, the use of even shorter wavelength radiation such as electron beams, X-rays, and extreme ultraviolet (EUV) rays is being considered. Resist compositions to which water-repellent compounds are added are required to have LWR (Line Width Roughness) performance, which indicates sensitivity and line width variation of resist patterns, water repellency, and development defect suppression properties, even in the next-generation technologies mentioned above.

However, if the solubility of the water-repellent compound in the developing solution is increased to improve development defect suppression, it may not be suitable for long-term storage due to its reactivity. There is a trade-off between storage stability and development defect suppression, and both are required.

According to the radiation-sensitive resin composition of an embodiment of the present disclosure, it is possible to construct a resist film that has good storage stability and satisfies sensitivity, LWR performance, water repellency, as well as development defect suppression. Although the reason for this is not certain and not limited and wishing not to be bound by a theory, it is inferred as follows. The polymer can exhibit high water repellency due to the fluorine atom contained in the structural unit (I). In addition, during the development process, a dissociation reaction occurs in the structural unit (I) in the polymer, and the solubility of the polymer in the developing solution can be improved, and as a result, the development defect suppression property can be improved. On the other hand, the relatively bulky structure introduced around the portion (mainly the ester bond at the end of the side chain) in the structural unit (I) where the dissociation reaction in the developing solution occurs acts as a reaction barrier, and an unintentional dissociation reaction due to moisture during storage of the radiation-sensitive resin composition can be suppressed. It is presumed that these combined effects can achieve both the required performance of storage stability and development defect suppression, which are in conflict with each other. In addition, it is also considered that the coexistence of the polymer and the onium salt (i) can ensure the sensitivity and LWR performance.

According to the patterning method of an embodiment of the present disclosure, a resist film with excellent sensitivity, LWR performance, water repellency, and development defect suppression can be formed, and since the above radiation-sensitive resin composition with good storage stability is used, high-grade resist patterns can be formed efficiently.

Hereinafter, embodiments of the present invention will be described in detail, but the present invention is not limited to these embodiments. Combinations of suitable embodiments are also preferable.

### <<Radiation-Sensitive Resin Composition>>

A radiation-sensitive resin composition (hereinafter, also simply referred to as a “composition”) according to the present embodiment contains a polymer, an onium salt (i), and a solvent. The composition preferably contains, in addition to the polymer, a resin (hereinafter, also referred to as a “base resin”) that contains a structural unit having an acid-dissociable group, and that has a lower mass content of fluorine atoms than the mass content of fluorine atoms of the polymer. The composition preferably further contains a radiation-sensitive acid generator. The composition may further contain another optional component as long as the effects of the present disclosure are not impaired.

### <Polymer>

The polymer contains a structural unit (I) represented by a formula (1) and a structural unit different from the structural unit (I). Hereinafter, each of the structural units will be described.

### (Structural Unit (I))

The structural unit (I) is represented by a formula (1).

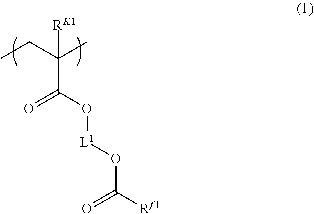

wherein, in formula (1),
R^{K1} is a hydrogen atom, a fluorine atom, a methyl group, or a trifluoromethyl group.
L¹ is an alkanediyl group having 1 to 5 carbon atoms.
R^{f1} is a monovalent fluorinated hydrocarbon group having 2 to 10 carbon atoms and 5 to 7 fluorine atoms.

R^{K1} is preferably a hydrogen atom or a methyl group in terms of copolymerizability of a monomer that gives the structural unit (I).

Examples of the alkanediyl group having 1 to 5 carbon atoms represented by L¹ include a group obtained by removing two hydrogen atoms from a chain or branched alkane with a carbon atom(s) as many as the corresponding number. The two hydrogen atoms may be removed from an identical carbon atom or different carbon atoms. Specific examples of the alkanediyl group having 1 to 5 carbon atoms include a methanediyl group, a 1,1-ethanediyl group, a 1,2-ethanediyl group, a 1,1-dimethyl-1,2-ethanediyl group, a 1,1-propanediyl group, a 1,2-propanediyl group, a 1,3-propanediyl group, a 2,2-propanediyl group, a 1,1-butanediyl group, a 2,2-butanediyl group, a 1,2-butanediyl group, a 1,3-butanediyl group, a 1,4-butanediyl group, and a 2,3-butanediyl group. Among these examples, L¹ is preferably a methanediyl group or an ethanediyl group (a 1,1-ethanediyl group or a 1,2-ethanediyl group).

Examples of the monovalent fluorinated hydrocarbon group with 5 to 7 fluorine atoms represented by R^{f1} include a monovalent fluorinated chain hydrocarbon group having 2 to 10 carbon atoms and 5 to 7 fluorine atoms, and a monovalent fluorinated alicyclic hydrocarbon group having 3 to 10 carbon atoms and 5 to 7 fluorine atoms.

Examples of the monovalent fluorinated chain hydrocarbon group having 2 to 10 carbon atoms and 5 to 7 fluorine atoms include:
fluorinated alkyl groups such as a pentafluoroethyl group, a 2,2,3,3,3-pentafluoroproyl group, a heptafluoropropyl group, a 1,1,1,3,3,3-hexafluoro-2-propyl group, a 1,1,1,3,3,3-hexafluoro-2-fluoropropyl group, and a heptafluoro-n-propyl group;
fluorinated alkenyl groups such as a pentafluoropropenyl group; and
fluorinated alkynyl groups such as a pentafluorobutynyl group.

Examples of the monovalent fluorinated alicyclic hydrocarbon group having 3 to 10 carbon atoms and 5 to 7 fluorine atoms include:
fluorinated cycloalkyl groups such as a pentafluorocyclobutyl group, a hexafluorocyclobutyl group, a pentafluorocyclopentyl group, a hexafluorocyclopentyl group, a heptafluorocyclopentyl group, a pentafluorocyclohexyl group, a hexafluorocyclohexyl group, a pentafluorocyclohexylmethyl group, a pentafluoronorbornyl group, a pentafluoroadamantyl group, a pentafluorobornyl group, and a pentafluoroisobornyl group; and
fluorinated cycloalkenyl groups such as a pentafluorocyclopentenyl group and a pentafluorocyclohexenyl group.

R^{f1} is preferably a monovalent fluorinated chain hydrocarbon group having 2 to 8 carbon atoms and 5 to 7 fluorine atoms, more preferably a monovalent fluorinated chain hydrocarbon group having 2 to 6 carbon atoms and 5 to 7 fluorine atoms, still more preferably a monovalent linear hydrocarbon group having 2 to 4 carbon atoms and 5 to 7 fluorine atoms, further preferably a monovalent linear hydrocarbon group having 2 to 4 carbon atoms and 5 fluorine atoms, and particularly preferably a monovalent linear saturated hydrocarbon group having 2 to 4 carbon atoms and 5 fluorine atoms.

Examples of the monomer that gives the structural unit (I) include compounds represented by formulas (1-1) to (1-18).

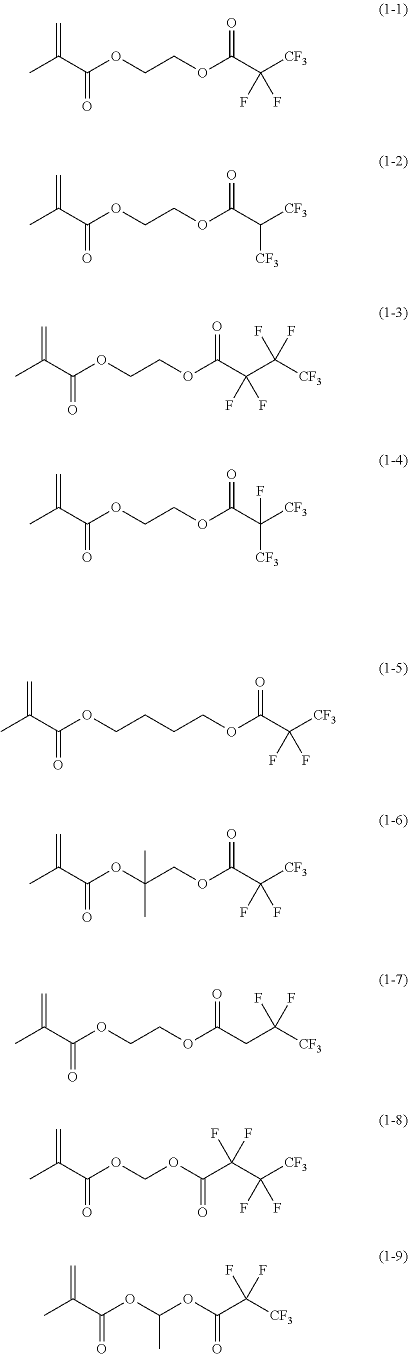

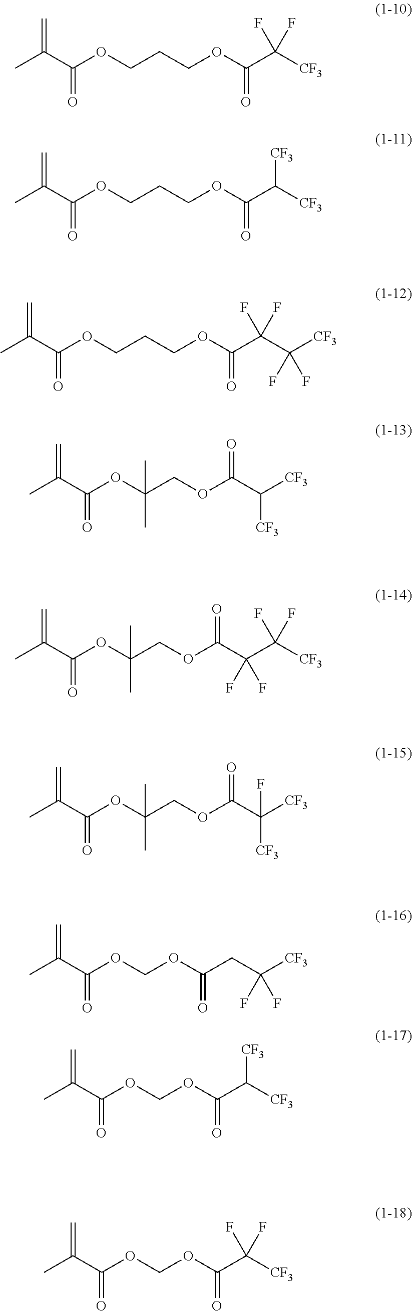

The lower limit of the content of the structural unit (I) (the total when a plurality of structural units (I) are present) in all the structural units constituting the polymer is preferably 5 mol %, more preferably 10 mol %, still more preferably 15 mol %, and particularly preferably 20 mol %. The upper limit of the content is preferably 95 mol %, more preferably 80 mol %, still more preferably 65 mol %, and particularly preferably 50 mol %. By setting the content of the structural unit (I) to the above range, the radiation-sensitive resin composition can further improve the water repellency, the storage stability, and the properties of suppressing development defects.

(Method for Synthesizing Monomer that Gives Structural Unit (I))

A monomer (α) that gives the structural unit (I) can, as illustrated by the following scheme, be synthesized through a condensation reaction between a polymerizable group-containing alcohol and a fluorine-containing carboxylic acid. Other structures can also be synthesized by changing the structure of the alcohol or the carboxylic acid.

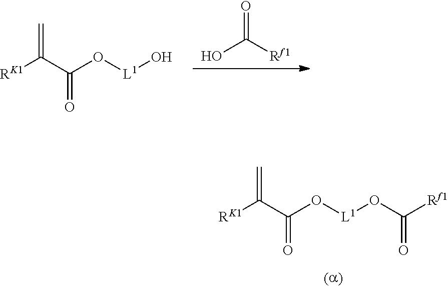

In the scheme, R^{K1}, L¹, and R^{f1} have the same definition as in formula (1).

### (Structural Unit (II))

The polymer preferably further contains, as the structural unit different from the structural unit (I), a structural unit (II) (except for the structure corresponding to the structural unit (I)) represented by a formula (2).

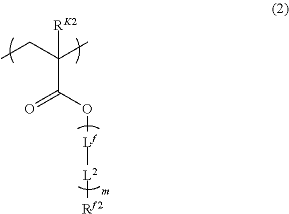

wherein, in formula (2),
R^{K2} is a hydrogen atom, a fluorine atom, a methyl group, or a trifluoromethyl group.
L^{f} is a fluorine-substituted or unsubstituted divalent organic group having 1 to 20 carbon atoms.
L² is *—COO— or *—OCO—. * is a bond on an L^{f} side.
m is an integer of 0 to 2. when a plurality of L^{f}s and Les are present, the plurality of L^{f}s and L²s are identical or different from each other.
R^{f2} is a fluorine-substituted or unsubstituted monovalent organic group having 1 to 20 carbon atoms.
L^{f} and R^{f2} contain one or more fluorine atoms in total.
R^{K2} is preferably a hydrogen atom or a methyl group in terms of copolymerizability of a monomer that gives the structural unit (II).

L^{f} is a divalent organic group having 1 to 20 carbon atoms, in which a part or all of the hydrogen atoms may be substituted with a fluorine atom or may not be substituted with a fluorine atom (may be unsubstituted). As the fluorine-substituted or unsubstituted divalent organic group having 1 to 20 carbon atoms represented by L^{f}, a group obtained by removing one hydrogen atom from the fluorine-substituted or unsubstituted monovalent organic group having 1 to 20 carbon atoms represented by R^{f2} in formula (2) can suitably be employed, and therefore R^{f2} will be described first.

R^{f2} is a monovalent organic group having 1 to 20 carbon atoms, in which a part or all of the hydrogen atoms may be substituted with a fluorine atom or may not be substituted with a fluorine atom (may be unsubstituted). The monovalent organic group having 1 to 20 carbon atoms is not particularly limited, and may have a chain structure, a cyclic structure, or a combination thereof. Examples of the chain structure include a chain hydrocarbon group that may either be saturated or unsaturated and linear or branched. Examples of the cyclic structure include a cyclic hydrocarbon group that may be alicyclic, aromatic, or heterocyclic. Among these examples, the monovalent organic group is preferably a substituted or unsubstituted monovalent chain hydrocarbon group having 1 to 20 carbon atoms, a substituted or unsubstituted monovalent alicyclic hydrocarbon group having 3 to 20 carbon atoms, a substituted or unsubstituted monovalent aromatic hydrocarbon group having 6 to 20 carbon atoms, or a combination thereof. Other examples include a group obtained by substituting, with a substituent, a part of all of the hydrogen atoms contained in a group having a chain structure or a group having a cyclic structure, and such a group but further containing, between carbon atoms or at a carbon-chain end, —CO—, —CS—, —O—, —S—, —SO₂—, —NR′—, or a combination of two or more thereof. R′ is a hydrogen atom or a C1-10 monovalent hydrocarbon group.

Examples of the substituent that substitutes a part or all of the hydrogen atoms contained in the organic group include halogen atoms such as a fluorine atom, a chlorine atom, a bromine atom, and an iodine atom, a hydroxy group, a carboxy group, a cyano group, a nitro group, an alkyl group, an alkoxy group, an alkoxycarbonyl group, an alkoxycarbonyloxy group, an acyl group, an acyloxy group, groups obtained by substituting a hydrogen atom of these groups with a halogen atom, and an oxo group (═O).

Examples of the monovalent chain hydrocarbon group having 1 to 20 carbon atoms include a linear or branched saturated hydrocarbon group having 1 to 20 carbon atoms, or a linear or branched unsaturated hydrocarbon group having 1 to 20 carbon atoms.

Examples of the monovalent alicyclic hydrocarbon group having 3 to 20 carbon atoms include a monocyclic or polycyclic saturated hydrocarbon group, or a monocyclic or polycyclic unsaturated hydrocarbon group. Preferred examples of the monocyclic saturated hydrocarbon groups include a cyclopentyl group, a cyclohexyl group, a cycloheptyl group, and a cyclooctyl group. The polycyclic cycloalkyl group is preferably a bridged alicyclic hydrocarbon group such as a norbornyl group, an adamantyl group, a tricyclodecyl group, or a tetracyclododecyl group. Examples of the monocyclic unsaturated hydrocarbon group include monocyclic cycloalkenyl groups such as a cyclopropenyl group, a cyclobutenyl group, a cyclopentenyl group, and a cyclohexenyl group. Examples of the polycyclic unsaturated hydrocarbon group include polycyclic cycloalkenyl groups such as a norbornenyl group, a tricyclodecenyl group, and a tetracyclododecenyl group. It is to be noted that the bridged alicyclic hydrocarbon group refers to a polycyclic alicyclic hydrocarbon group in which two carbon atoms that constitute an alicyclic ring and are not adjacent to each other are bonded by a bonding chain containing one or more carbon atoms.

Examples of the monovalent aromatic hydrocarbon group having 6 to 20 carbon atoms include aryl groups such as a phenyl group, a tolyl group, a xylyl group, a naphthyl group, and an anthryl group; and aralkyl groups such as a benzyl group, a phenethyl group, and a naphthylmethyl group.

Examples of the heterocyclic cyclic hydrocarbon group include a group obtained by removing one hydrogen atom from an aromatic heterocyclic structure, and a group obtained by removing one hydrogen atom from an alicyclic heterocyclic structure. A 5-membered aromatic structure having aromaticity is also included in the heterocyclic structure when having a heteroatom introduced therein. Examples of the heteroatom include an oxygen atom, a nitrogen atom, and a sulfur atom.

Examples of the aromatic heterocyclic structure include:
oxygen atom-containing aromatic heterocyclic structures such as furan, pyran, benzofuran, and benzopyran;
nitrogen atom-containing aromatic heterocyclic structures such as pyrrole, imidazole, pyridine, pyrimidine, pyrazine, indole, quinoline, isoquinoline, acridine, phenazine, and carbazole;
sulfur atom-containing aromatic heterocyclic structure such as thiophene; and
aromatic heterocyclic structures containing a plurality of heteroatoms, such as thiazole, benzothiazole, thiazine, and oxazine.

Examples of the aliphatic heterocyclic structure include:
oxygen atom-containing alicyclic heterocyclic structures such as oxirane, tetrahydrofuran, tetrahydropyran, dioxolane, and dioxane;
nitrogen atom-containing alicyclic heterocyclic structures such as aziridine, pyrrolidine, piperidine, and piperazine;
sulfur atom-containing alicyclic heterocyclic structures such as thietane, thiolane, and thiane; and
alicyclic heterocyclic structures containing a plurality of heteroatoms, such as morpholine, 1,2-oxathiolane, and 1,3-oxathiolane;
a lactone structure, a cyclic carbonate structure, and a sultone structure;
a spiro heterocyclic structure in which a plurality of the aliphatic heterocyclic structures are bonded by sharing a quaternary carbon atom.

Examples of the cyclic structure also include a lactone structure, a cyclic carbonate structure, a sultone structure, and a structure containing a cyclic acetal.

As the fluorine-substituted or unsubstituted divalent organic group having 1 to 20 carbon atoms represented by L^{f}, a group obtained by removing one hydrogen atom from the fluorine-substituted or unsubstituted monovalent organic group having 1 to 20 carbon atoms represented by R^{f2} can suitably be employed as described above.

m is preferably 1 or 2, and more preferably 1.

L^{f} and R^{f2} contain a total of one or more fluorine atoms. The lower limit of the total number of fluorine atoms contained in L^{f} and R^{f2} may be 2 or 3. The upper limit of the total number may be 10, 8, or 6.

In formula (2), one or two fluorine atoms, or a trifluoromethyl group is preferably bonded to at least one carbon atom adjacent to the carbonyl group of L².

Examples of the monomer that gives the structural unit (II) include compounds represented by formulas (2-1) to (2-42).

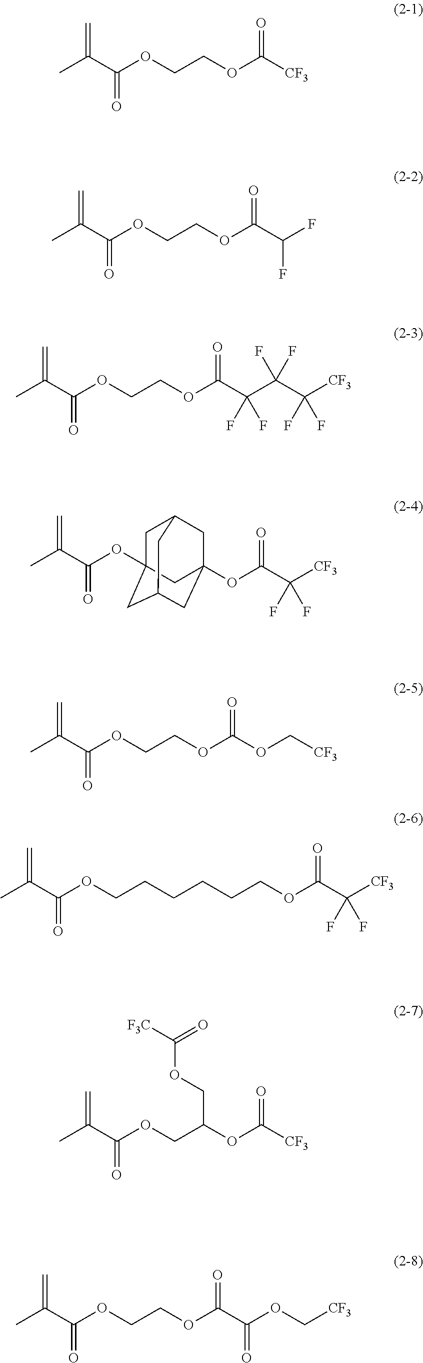

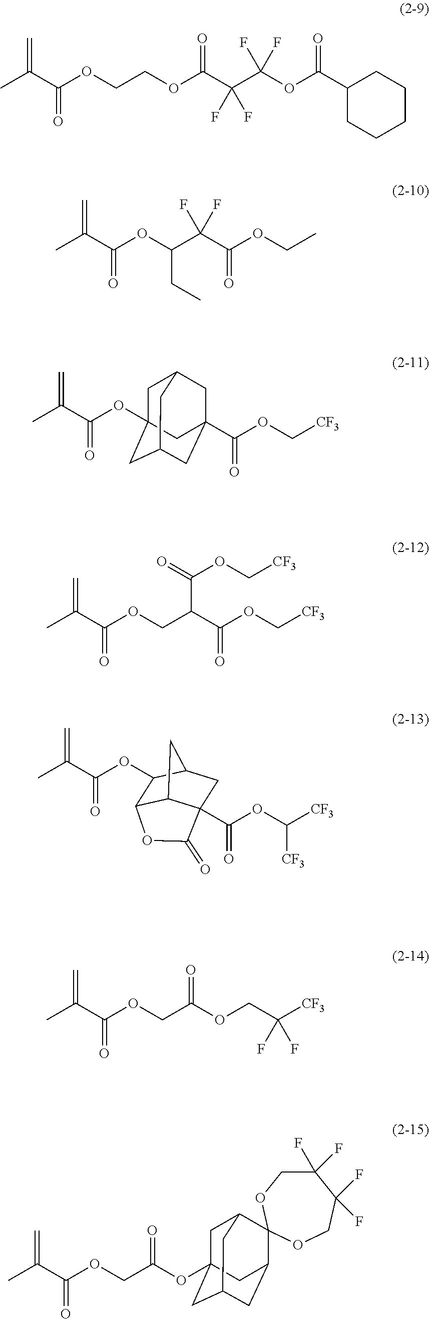

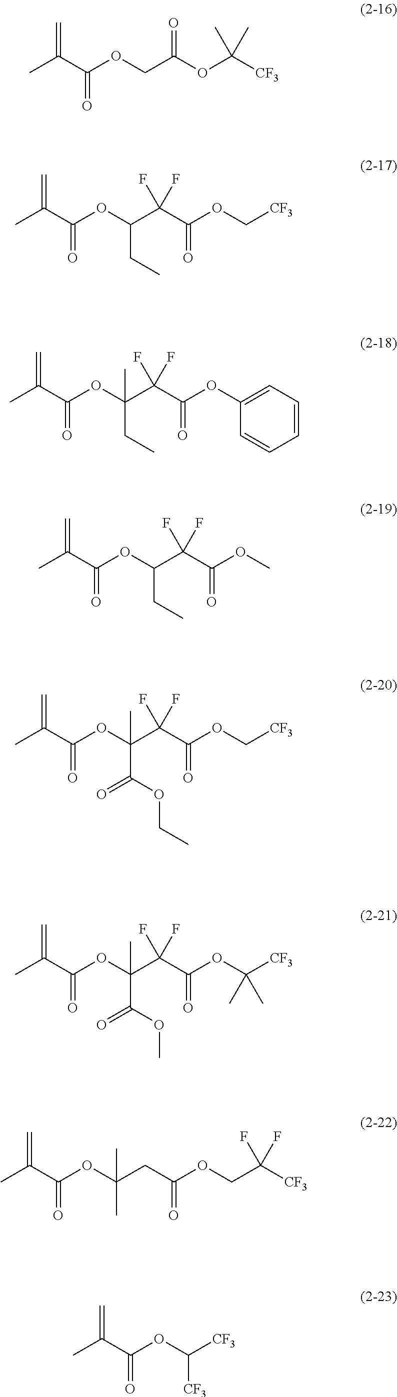

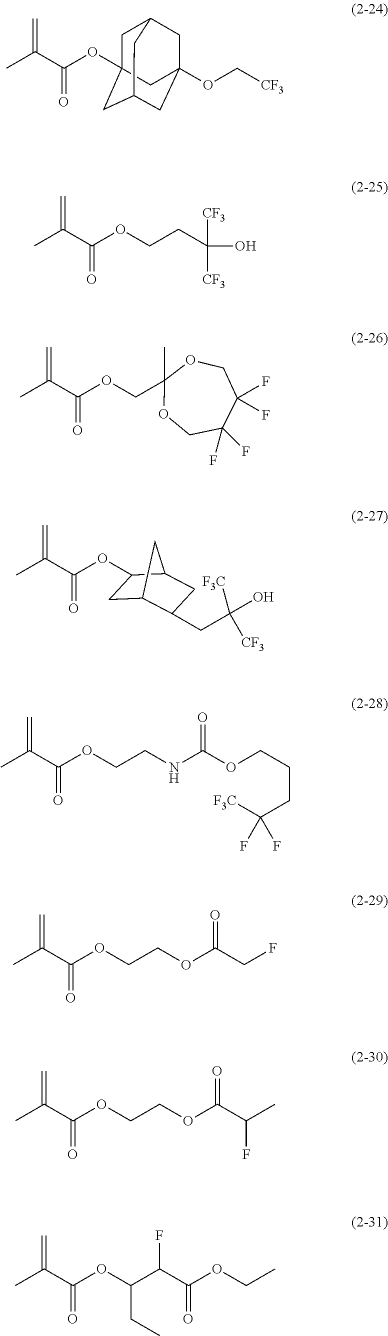

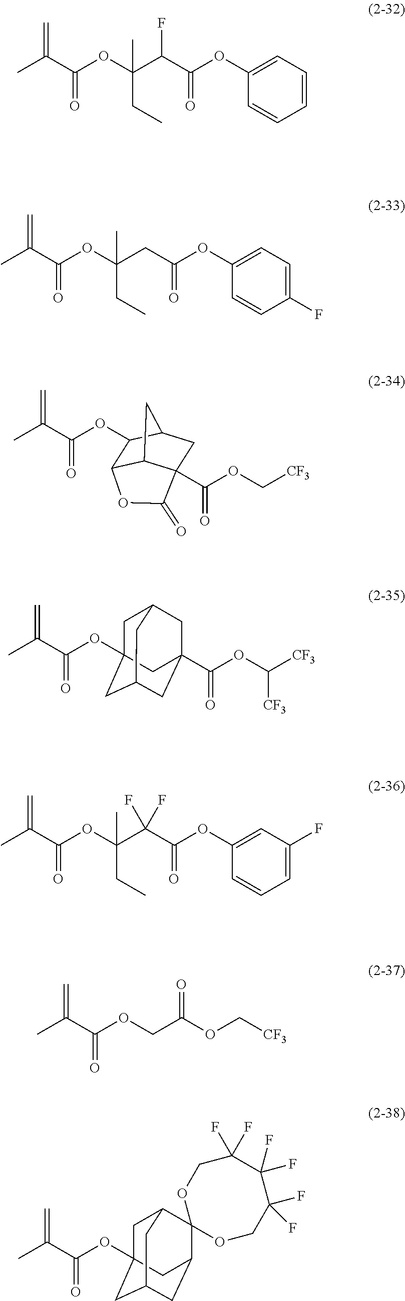

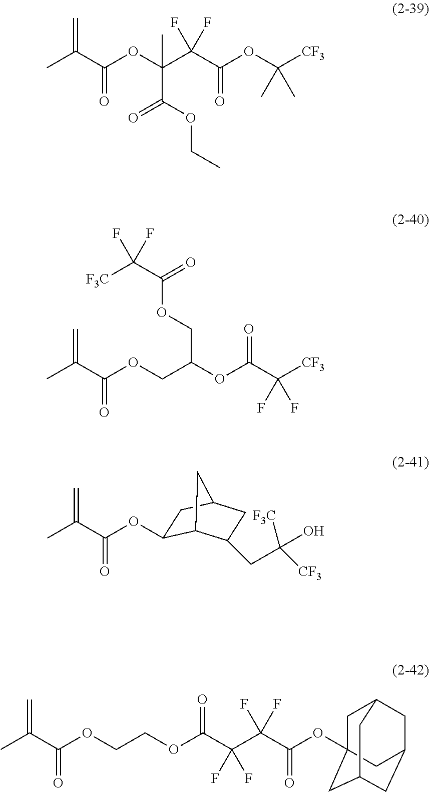

The lower limit of the content of the structural unit (II) (the total when a plurality of structural units (II) are present) in all the structural units constituting the polymer is preferably 5 mol, more preferably 10 mol, still more preferably 15 mol %, and particularly preferably 20 mol %. The upper limit of the content is preferably 80 mol %, more preferably 70 mol %, still more preferably 60 mol %, and particularly preferably 50 mol %. By setting the content of the structural unit (II) to the above range, the radiation-sensitive resin composition can further improve the water repellency, the storage stability, and the properties of suppressing development defects.

### (Structural Unit (III))

The polymer preferably further contains, as the structural unit different from the structural unit (I), a structural unit (III) represented by a formula (3).

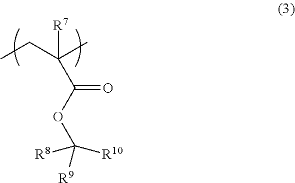

In formula (3),
R⁷ is a hydrogen atom, a fluorine atom, a methyl group, or a trifluoromethyl group.
R⁸ is a monovalent hydrocarbon group having 1 to 20 carbon atoms.
R⁹ and R¹⁰ each independently represent a monovalent chain hydrocarbon group having 1 to 10 carbon atoms or a monovalent alicyclic hydrocarbon group having 3 to 20 carbon atoms, or R⁹ and R¹⁰ taken together represent a divalent alicyclic group having 3 to 20 carbon atoms together with the carbon atom to which R⁹ and R¹⁰ are bonded.

From the viewpoint of copolymerizability of a monomer that gives the structural unit (III), R⁷ is preferably a hydrogen atom or a methyl group, and more preferably a methyl group.

Examples of the monovalent hydrocarbon group having 1 to 20 carbon atoms represented by R⁸ include a monovalent chain hydrocarbon group having 1 to 10 carbon atoms, a monovalent alicyclic hydrocarbon group having 3 to 20 carbon atoms, and a monovalent aromatic hydrocarbon group having 6 to 20 carbon atoms.

As the monovalent chain hydrocarbon groups having 1 to 10 carbon atoms represented by R⁸ to R¹⁰, the groups having 1 to 10 carbon atoms among the monovalent chain hydrocarbon groups having 1 to 20 carbon atoms of R^{f2} in formula (2) can suitably be employed.

As the monovalent alicyclic hydrocarbon groups having 3 to 20 carbon atoms represented by R⁸ to R¹⁰, the monovalent alicyclic hydrocarbon groups having 3 to 20 carbon atoms of R^{f2} in formula (2) can suitably be employed.

As the monovalent aromatic hydrocarbon group having 6 to 20 carbon atoms represented by R⁸, the monovalent aromatic hydrocarbon groups having 6 to 20 carbon atoms of R^{f2} in formula (2) can suitably be employed.

R⁸ is preferably a monovalent linear or branched saturated hydrocarbon group having 1 to 10 carbon atoms or a monovalent aromatic hydrocarbon group having 6 to 20 carbon atoms.

The divalent alicyclic group having 3 to 20 carbon atoms formed by R⁹ and R¹⁰ taken together with the carbon atom to which R⁹ and R¹⁰ are bonded is not particularly limited as long as the divalent alicyclic group having 3 to 20 carbon atoms is a group obtained by removing two hydrogen atoms from an identical carbon atom constituting a carbon ring of a monocyclic or polycyclic alicyclic hydrocarbon with carbon atoms as many as the aforementioned number. The divalent alicyclic group having 3 to 20 carbon atoms may be either a monocyclic hydrocarbon group or a polycyclic hydrocarbon group, may be either a bridged alicyclic hydrocarbon group or a condensed alicyclic hydrocarbon group when being a polycyclic hydrocarbon group, and may be either a saturated hydrocarbon group or an unsaturated hydrocarbon group. The condensed alicyclic hydrocarbon group refers to a polycyclic alicyclic hydrocarbon group that contains a plurality of alicyclic rings sharing a side (bond between two adjacent carbon atoms).

Among the monocyclic alicyclic hydrocarbon groups, the saturated hydrocarbon group is preferably a cyclopentanediyl group, a cyclohexanediyl group, a cycloheptanediyl group, a cyclooctanediyl group, or the like, and the unsaturated hydrocarbon group is preferably a cyclopentenediyl group, a cyclohexenediyl group, a cycloheptenediyl group, a cyclooctenediyl group, a cyclodecenediyl group, or the like. The polycyclic alicyclic hydrocarbon group is preferably a bridged alicyclic saturated hydrocarbon group, and preferred examples thereof include a bicyclo[2.2.1]heptane-2,2-diyl group (norbornane-2,2-diyl group), a bicyclo[2.2.2]octane-2,2-diyl group, and a tricyclo[3.3.1.1^{3,7}]decane-2,2-diyl group (adamantane-2,2-diyl group).

Among these examples, it is preferable that R⁸ is a alkyl group having 1 to 4 carbon atoms or a phenyl group, and the alicyclic structure containing R⁹ and R¹⁰ combined with each other, and a carbon atom to which these are bonded is a polycyclic or monocyclic cycloalkane structure.

Examples of the structural unit (III) include structural units represented by formulas (3-1) to (3-7) (hereinafter, also referred to as “structural units (III-1) to (III-7)”).

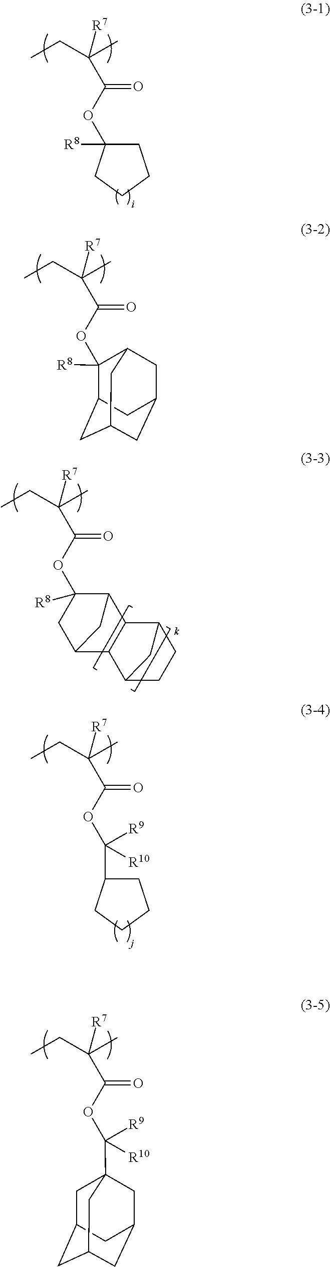

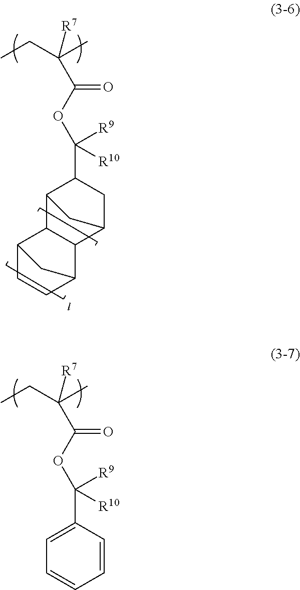

In formulas (3-1) to (3-7), R⁷ to R¹⁰ have the same definition as in formula (3). i and j are each independently an integer of 1 to 4. k and l are 0 or 1.

i and j are preferably 1. R⁸ is preferably a methyl group, an ethyl group, an isopropyl group, or a phenyl group. R⁹ and R¹⁰ are preferably a methyl group or an ethyl group.

The polymer may contain one structural unit (III) or two or more structural units (III) in combination.

The lower limit of the content of the structural unit (III) (the total when a plurality of structural units (III) are present) in all the structural units constituting the polymer is preferably 5 mol %, more preferably 8 mol %, still more preferably 10 mol %, and particularly preferably 15 mol %. The upper limit of the content is preferably 80 mol %, more preferably 75 mol %, still more preferably 70 mol %, and particularly preferably 65 mol %. By setting the content of the structural unit (III) to the above range, the radiation-sensitive resin composition can further improve the sensitivity, the LWR performance, and the properties of suppressing development defects.

### (Structural Unit (IV))

The polymer may contain, as the structural unit different from the structural unit (I), a structural unit (IV) (except for the structure corresponding to the structural unit (II)) containing at least one selected from the group consisting of a lactone structure, a cyclic carbonate structure, and a sultone structure. When the polymer further contain the structural unit (IV), the solubility of the polymer in a developer can be adjusted, and as a result, the radiation-sensitive resin composition can improve the lithographic performance such as resolution.

Examples of the structural unit (IV) include structural units represented by formulas (T-1) to (T-10).

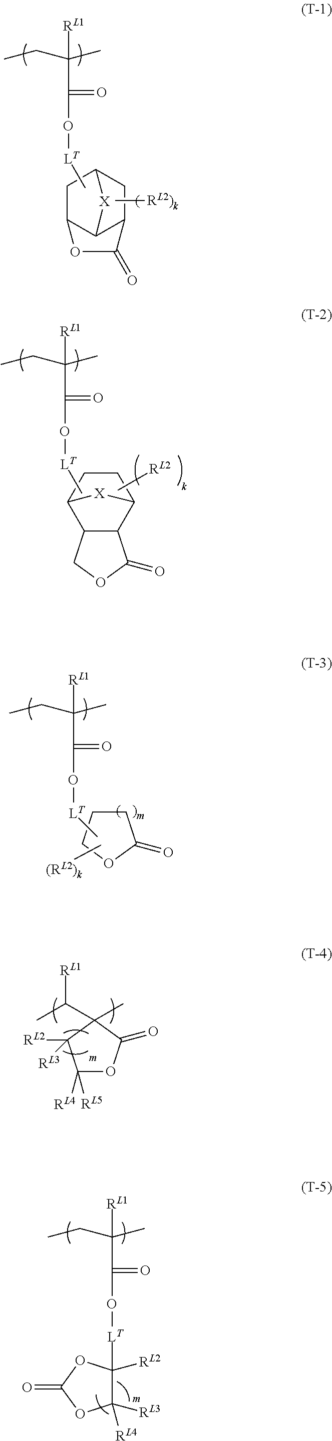

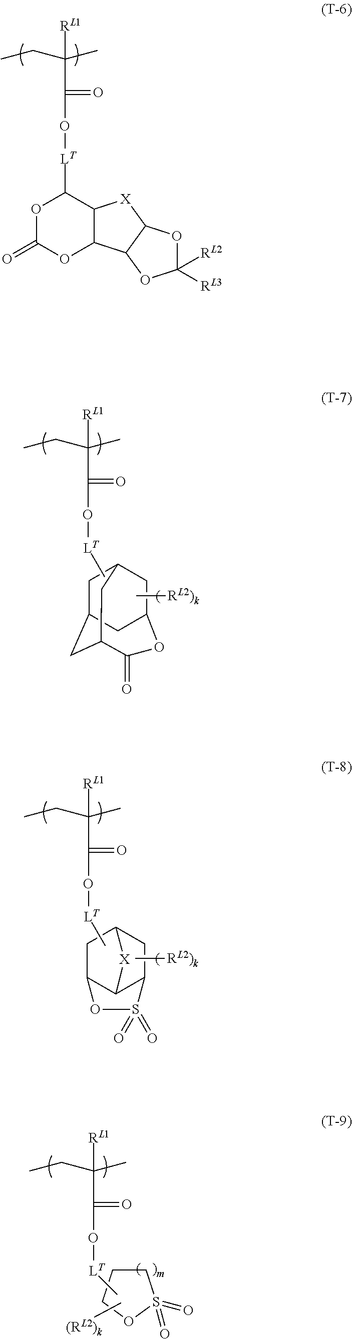

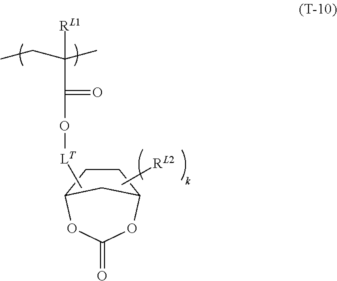

In formulas, R^{L1} is a hydrogen atom, a fluorine atom, a methyl group, or a trifluoromethyl group. R^{L2} to R^{L5} are each independently a hydrogen atom, a alkyl group having 1 to 4 carbon atoms, a cyano group, a trifluoromethyl group, a methoxy group, a methoxycarbonyl group, a hydroxy group, a hydroxymethyl group, or a dimethylamino group. R^{L4} and R^{L5} may be a having 1 to 10 carbon atoms divalent alicyclic group having 3 to 8 carbon atoms formed by R^{L4} and R^{L5} taken together with the carbon atom to which R^{L4} and R^{L5} are bonded. L^{T} is a single bond or a divalent linking group. X is an oxygen atom or a methylene group. k is an integer of 0 to 3. m is an integer of 1 to 3.

Examples of the divalent alicyclic group having 3 to 8 carbon atoms formed by R^{L4} and R^{L5} taken together with the carbon atom to which R^{L4} and R^{L5} are bonded include groups having 3 to 8 carbon atoms among the divalent alicyclic groups having 3 to 20 carbon atoms formed by R⁹ and R¹⁰ taken together with the carbon atom to which R⁹ and R¹⁰ are bonded in formula (3). One or more hydrogen atoms on this alicyclic group may be substituted with a hydroxy group.

Examples of the divalent linking group represented by L^{T} include a divalent linear or branched hydrocarbon group having 1 to 10 carbon atoms, a divalent alicyclic hydrocarbon group having 4 to 12 carbon atoms, and a group containing one or more of these hydrocarbon groups and at least one group of —CO—, —O—, —NH—, and —S—.

Among these examples, the structural unit (IV) is preferably a structural unit containing a lactone structure, more preferably a structural unit containing a norbornane lactone structure, and still more preferably a structural unit derived from norbornane lactone-yl (meth)acrylate.

The lower limit of the content of the structural unit (IV) (the total when a plurality of structural units (IV) are present) is preferably 5 mol %, more preferably 8 mol %, and still more preferably 10 mol %, with respect to all the structural units constituting the polymer. The upper limit of the content is preferably 50 mol %, more preferably 40 mol %, and still more preferably 35 mol %. By setting the content of the structural unit (IV) to the above range, the radiation-sensitive resin composition can further improve the lithographic performance such as resolution.

### (Structural Unit (V))

The polymer may contain, as the structural unit different from the structural unit (I), for example, a structural unit (V) (except for the structures corresponding to the structural units (II) and (IV)) containing a polar group. When the polymer further contains the structural unit (V), the solubility of the polymer in a developer can be adjusted, and as a result, the lithographic performance, such as resolution, of the radiation-sensitive resin composition can be improved. Examples of the polar group include a hydroxy group, a carboxy group, a cyano group, a nitro group, and a sulfonamide group. Among these examples, a hydroxy group and a carboxy group are preferable, and a hydroxy group is more preferable.

Examples of the structural unit (V) include structural units represented by formulas below.

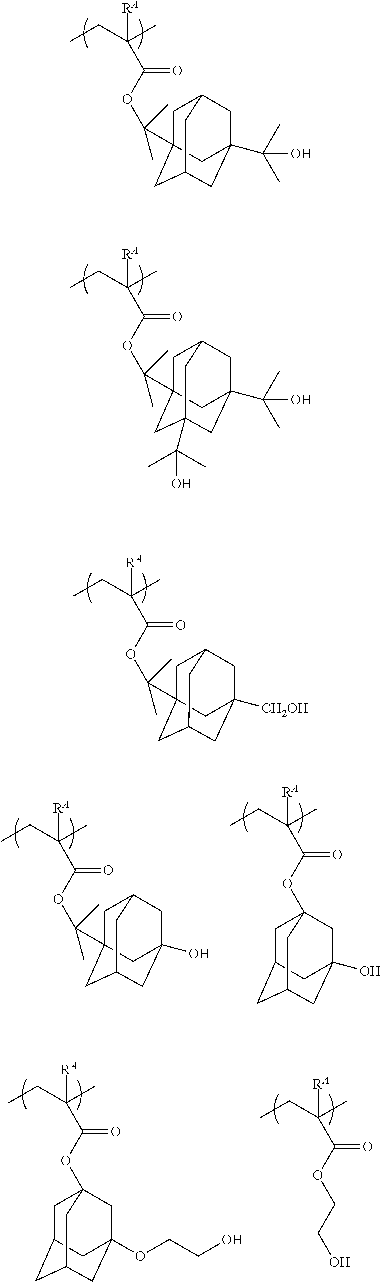

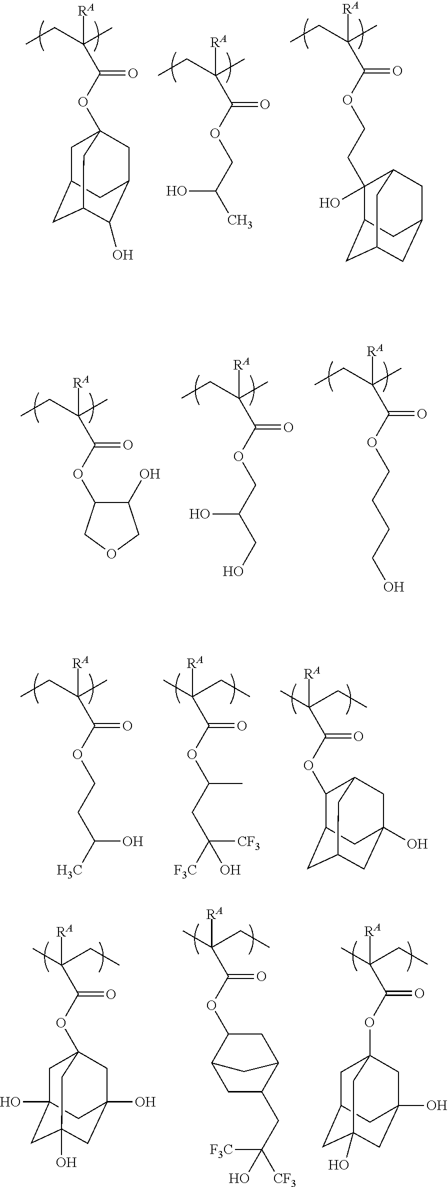

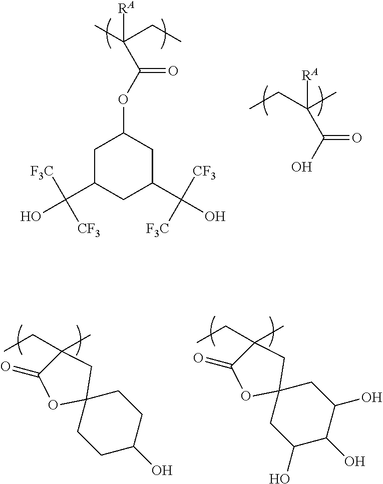

In formulas, R^{A} is a hydrogen atom, a fluorine atom, a methyl group, or a trifluoromethyl group.

When the polymer contains the structural unit (V) having a polar group, the lower limit of the content of the structural unit (V) (the total when a plurality of structural units (V) are present) is preferably 5 mol %, more preferably 8 mol %, and still more preferably 10 mol %, with respect to all the structural units constituting the polymer. The upper limit of the content is preferably 60 mol %, more preferably 50 mol %, and still more preferably 45 mol %. By setting the content of the structural unit (V) to the above range, the lithographic performance, such as resolution, of the radiation-sensitive resin composition can further be improved.

### (Other Structural Unit)

The polymer may contain, as a structural unit other than the structural units listed above, a structural unit represented by a formula (6) and containing an alicyclic structure.

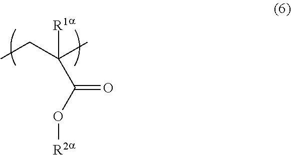

(In formula (6), R^{1α} is a hydrogen atom, a fluorine atom, a methyl group, or a trifluoromethyl group. R^{2α} is a monovalent alicyclic hydrocarbon group having 3 to 20 carbon atoms.)

As the monovalent alicyclic hydrocarbon group having 3 to 20 carbon atoms represented by R^{2α} in formula (6), the monovalent alicyclic hydrocarbon group having 3 to 20 carbon atoms represented by R⁸ to R¹⁰ in formula (3) can suitably be employed.

When the polymer contains the structural unit having an alicyclic structure, the lower limit of the content of the structural unit having an alicyclic structure is preferably 5 mol %, more preferably 10 mol %, and still more preferably 15 mol %, with respect to all the structural units constituting the polymer. The upper limit of the content is preferably 40 mol %, more preferably 30 mol %, and still more preferably 20 mol %.

### [Method for Synthesizing Polymer]

The polymer can be synthesized, for example, by subjecting the monomers, which give the structural units, to a polymerization reaction in an appropriate solvent, using a radical polymerization initiator or the like.

Examples of the radical polymerization initiator include: azo radical initiators such as azobisisobutyronitrile (AIBN), 2,2′-azobis(4-methoxy-2,4-dimethylvaleronitrile), 2,2′-azobis(2-cyclopropylpropionitrile), 2,2′-azobis(2,4-dimethylvaleronitrile), and dimethyl 2,2′-azobisisobutyrate; and peroxide radical initiators such as benzoyl peroxide, t-butyl hydroperoxide, and cumene hydroperoxide. Among these examples, AIBN and dimethyl 2,2′-azobisisobutyrate are preferable, and AIBN is more preferable. These radical initiators can be used singly or in mixture of two or more thereof.

Examples of the solvent used in the polymerization reaction include: alkanes such as n-pentane, n-hexane, n-heptane, n-octane, n-nonane, and n-decane; cycloalkanes such as cyclohexane, cycloheptane, cyclooctane, decalin, and norbornane; aromatic hydrocarbons such as benzene, toluene, xylene, ethylbenzene, and cumene; halogenated hydrocarbons such as chlorobutanes, bromohexanes, dichloroethanes, hexamethylene dibromide, and chlorobenzene; saturated carboxylic acid esters such as ethyl acetate, n-butyl acetate, i-butyl acetate, and methyl propionate; ketones such as acetone, methyl ethyl ketone, 4-methyl-2-pentanone, and 2-heptanone; ethers such as tetrahydrofuran, dimethoxyethanes, and diethoxyethanes; and alcohols such as methanol, ethanol, 1-propanol, 2-propanol, and 4-methyl-2-pentanol. These solvents used in the polymerization reaction may be used singly or in combination of two or more thereof.

The reaction temperature in the polymerization reaction is usually 40° C. to 150° C., and preferably 50° C. to 120° C. The reaction time is usually 1 hour to 48 hours, and preferably 1 hour to 24 hours.

The molecular weight of the polymer is not particularly limited, but the lower limit of the weight-average molecular weight (Mw) as determined by gel permeation chromatography (GPC) relative to standard polystyrene is preferably 3,000, more preferably 5,000, still more preferably 6,000, and particularly preferably 7,000. The upper limit of the Mw is preferably 30,000, more preferably 20,000, still more preferably 15,000, and particularly preferably 12,000. By setting the Mw of the polymer to the above range, the storage stability and the properties of suppressing development defects of the radiation-sensitive resin composition can be improved.

The ratio (Mw/Mn) of the Mw to the number-average molecular weight (Mn) of the polymer as determined by GPC relative to standard polystyrene is usually 1 or more and 5 or less, preferably 1 or more and 3 or less, and still more preferably 1 or more and 2 or less.

The method for measuring the Mw and the Mn of the polymer and a base resin described later is as described in Examples.

The lower limit of the content amount of the polymer is preferably 0.1 parts by mass, more preferably 0.5 parts by mass, still more preferably 1 part by mass, and particularly preferably 2 parts by mass, with respect to 100 parts by mass of the base resin described later. The upper limit of the content amount is preferably 40 parts by mass, more preferably 30 parts by mass, still more preferably 20 parts by mass, and particularly preferably 15 parts by mass.

### <Base Resin>

The base resin is a resin that contains a structural unit having an acid-dissociable group, and that has a smaller mass content of fluorine atoms than the mass content of fluorine atoms of the polymer. As the structural unit, in the base resin, having an acid-dissociable group, the structural unit (III) contained in the polymer can suitably be employed. (Hereinafter, referred to as a “structural unit (III)” also in the base resin. The same applies to the other structural units.) When the base resin contains the structural unit (III), the radiation-sensitive resin composition has excellent patternability.

The lower limit of the content of the structural unit (III) (the total when a plurality of structural units (III) are present) in all the structural units constituting the base resin is preferably 15 mol %, more preferably 25 mol %, still more preferably 30 mol %, and particularly preferably 35 mol %. The upper limit of the content is preferably 80 mol %, more preferably 75 mol %, still more preferably 70 mol %, and particularly preferably 65 mol %. By setting the content of the structural unit (III) in the base resin to the above range, the patternability of the radiation-sensitive resin composition can further be improved.

The base resin may also contain the structural unit (IV) or (V) of the polymer in addition to the structural unit (III) having an acid-dissociable group. Further, the base resin may also contain a structural unit derived from hydroxystyrene or a structural unit containing a phenolic hydroxy group (hereinafter, also referred to as a “structural unit (VI)”, described later).

When the base resin contains the structural unit (IV), the lower limit of the content of the structural unit (IV) is preferably 20 mol %, more preferably 30 mol %, and still more preferably 35 mol %, with respect to all the structural units constituting the base resin. The upper limit of the content is preferably 80 mol %, more preferably 70 mol %, and still more preferably 65 mol %. By setting the content of the structural unit (IV) to the above range, the radiation-sensitive resin composition can further improve the lithographic performance, such as resolution, and the adhesion between the resist pattern formed and the substrate.

When the base resin contains the structural unit (V), the lower limit of the content of the structural unit (V) is preferably 5 mol %, more preferably 8 mol %, and still more preferably 10 mol %, with respect to all the structural units constituting the base resin. The upper limit of the content is preferably 40 mol %, more preferably 30 mol %, and still more preferably 20 mol %. By setting the content of the structural unit (V) to the above range, the lithographic performance, such as resolution, of the radiation-sensitive resin composition can further be improved.

### (Structural Unit (VI))

The structural unit (VI) is a structural unit derived from hydroxystyrene or a structural unit containing a phenolic hydroxy group. The structural unit (VI) contributes to improvement of etching resistance and improvement of a difference in solubility to a developer between an exposed area and an unexposed area (dissolution contrast). In particular, the structural unit (VI) can suitably be applied to pattern formation using exposure with a radiation having a wavelength of 50 nm or less, such as an electron beam and EUV. For the exposure with a radiation having a wavelength of 50 nm or less, the base resin preferably contains the structural unit (VI) as well as the structural unit (III), and a structural unit (V) as desired.

The structural unit derived from hydroxystyrene is represented by, for example, formulas (4-1) and (4-2), and the structural unit containing a phenolic hydroxy group is represented by, for example, formulas (4-3) and (4-4).

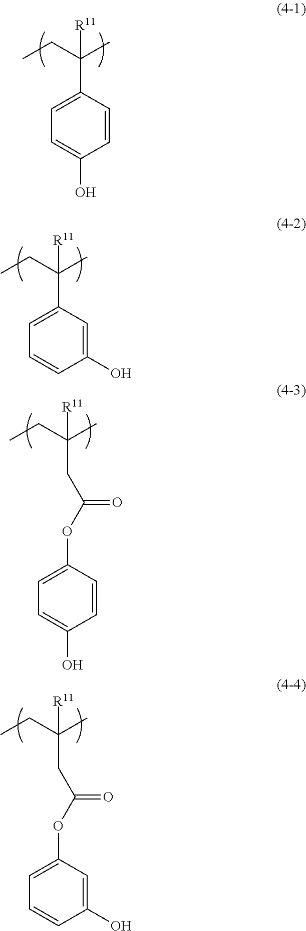

In formulas (4-1) to (4-4), R¹¹ is a hydrogen atom, a fluorine atom, a methyl group, or a trifluoromethyl group.

When the structural unit (VI) is obtained, it is preferable to polymerize a monomer with the phenolic hydroxy group protected by a protecting group such as an alkali-dissociable group (e.g., an acyl group) during the polymerization, and then deprotect the polymerized product by hydrolysis to obtain the structural unit (VI).

When the base resin is for the exposure with a radiation having a wavelength of 50 nm or less, the lower limit of the content of the structural unit (VI) is preferably 10 mol %, and more preferably 20 mol %, with respect to all the structural units constituting the base resin. The upper limit of the content is preferably 70 mol %, and more preferably 60 mol %.

The molecular weight of the base resin is not particularly limited, and the lower limit of the weight-average molecular weight (Mw) as determined by gel permeation chromatography (GPC) relative to standard polystyrene is preferably 1,000, more preferably 2,000, still more preferably 3,000, and particularly preferably 4,000. The upper limit of the Mw is preferably 30,000, more preferably 20,000, still more preferably 12,000, and particularly preferably 8,000. By setting the Mw of the base resin to the above range, the resist film obtained can have good heat resistance and developability.

The ratio (Mw/Mn) of the Mw to the number-average molecular weight (Mn) of the base resin as determined by GPC relative to standard polystyrene is usually 1 or more and 5 or less, preferably 1 or more and 3 or less, and more preferably 1 or more and 2 or less.

### (Method for Synthesizing Base Resin)

The base resin can be synthesized by the same method as the method, described above, for synthesizing the polymer.

<Onium Salt (i)>

The onium salt (i) is represented by a formula (i). By employing the onium salt (i), the radiation-sensitive resin composition has excellent storage stability. The radiation-sensitive resin composition contains the onium salt (i) as a quencher (acid diffusion controlling agent) and can therefore exhibit excellent LWR performance and properties of suppressing development defects in the pattern formation.

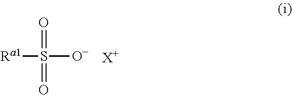

wherein, in formula (i),
R^{a1} is a substituted or unsubstituted monovalent organic group having 1 to 40 carbon atoms with no fluorine atom or fluorinated hydrocarbon group attached to an atom adjacent to the sulfur atom.
X⁺ is a monovalent onium cation.)

As the monovalent organic group having 1 to 40 carbon atoms represented by R^{a1}, the monovalent organic groups of R^{f2} in formula (2) having, instead of 1 to 20 carbon atoms, an expanded range of carbon atoms of up to 40 can suitably be employed. As a substituent of the organic group of R^{a1}, the substituents that can be contained in the organic group of R^{f2} in formula (2) can suitably be employed. However, the atom of R^{a1} adjacent to the sulfur atom in formula (i) needs to have no fluorine atom and no fluorinated hydrocarbon group bonded thereto.

R^{a1} preferably contains a cyclic structure. By the cyclic structure, the radiation-sensitive resin composition can exhibit excellent sensitivity, LWR performance, and properties of suppressing development defects in the pattern formation. As the cyclic structure, the cyclic structures that can be contained in the organic group of R^{f2} in formula (2) can suitably be employed. Especially, the cyclic structure is preferably a C5-15 monocyclic or polycyclic alicyclic hydrocarbon structure, an aromatic hydrocarbon structure having 6 to 10 carbon atoms, a lactone structure, a sultone structure, or a combination thereof.

An anion moiety of the onium salt (i) represented by the formula (i) is not particularly limited, and examples thereof include structures represented by formulas (i-1-1) to (i-1-27).

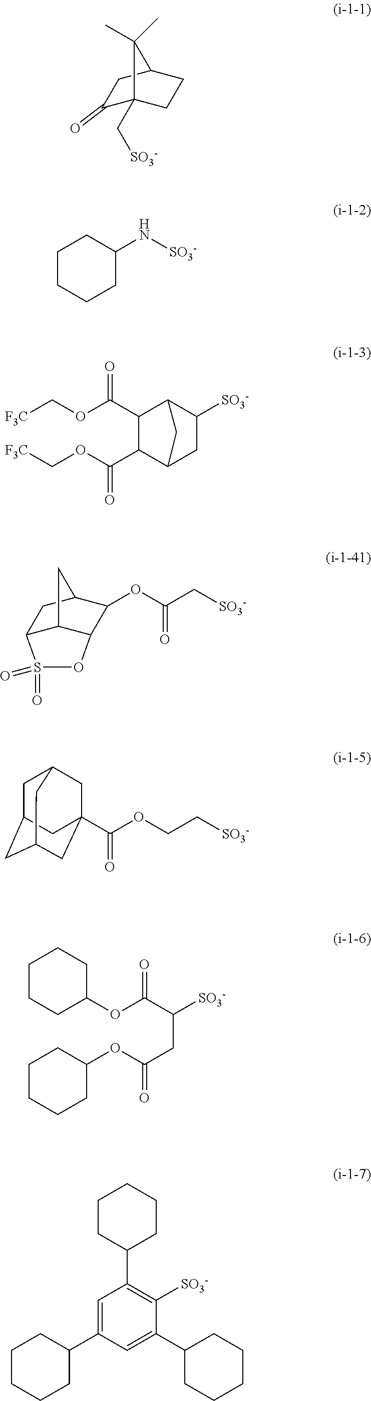

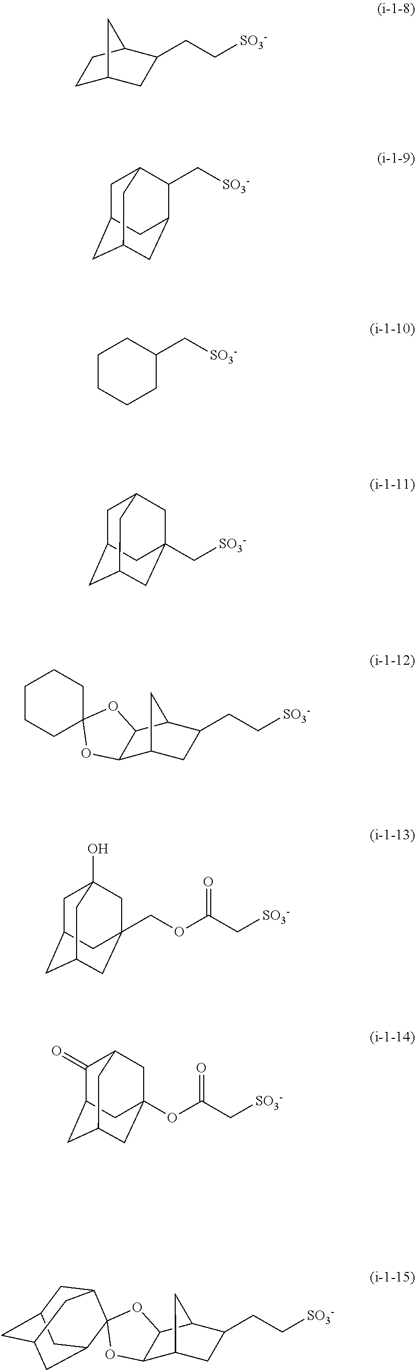

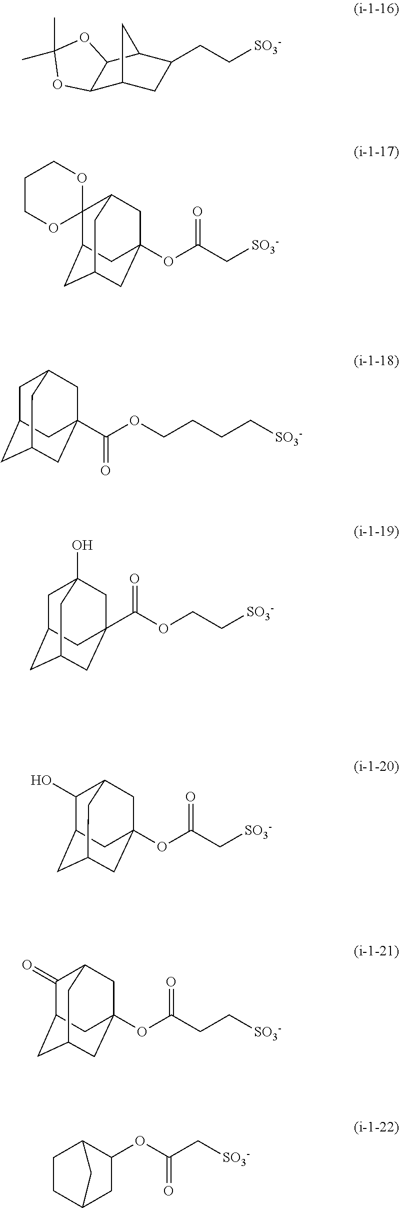

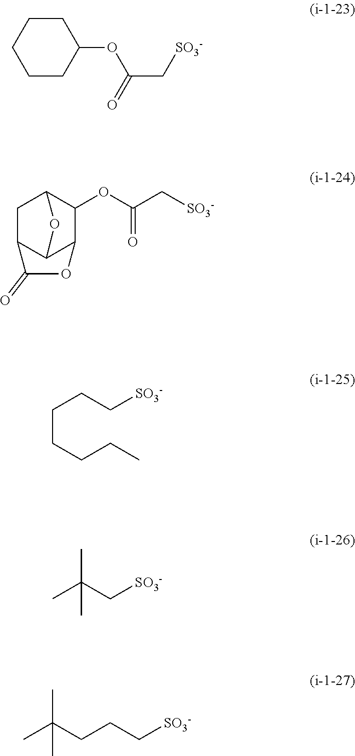

Examples of the monovalent onium cation represented by X⁺ in formula (i) include an onium cation containing an element such as S, I, O, N, P, Cl, Br, F, As, Se, Sn, Sb, Te, and Bi. Examples of the onium cation include a sulfonium cation, a tetrahydrothiophenium cation, an iodonium cation, a phosphonium cation, a diazonium cation, a pyridinium cation, and an ammonium cation. Among these examples, a sulfonium cation or an iodonium cation is preferable. The sulfonium cation and the iodonium cation are preferably represented by formulas (X-1) to (X-6).

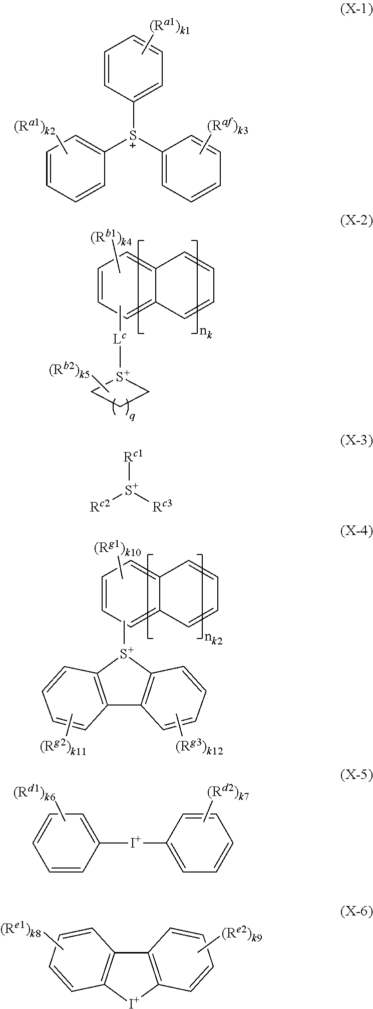

In the above formula (X-1), R^{a1}, R^{a2} and R^{a3} are each independently a substituted or unsubstituted, straight or branched chain alkyl group, alkoxy group or alkoxycarbonyloxy group having a carbon number of 1 to 12; a substituted or unsubstituted, monocyclic or polycyclic cycloalkyl group having a carbon number of 3 to 12; a substituted or unsubstituted aromatic hydrocarbon group having a carbon number of 6 to 12; a hydroxy group, a halogen atom, —OSO₂—R^{P}, —SO₂—R^{Q} or —S—R^{T}; or a ring structure obtained by combining two or more of these groups. The ring structure may contain heteroatoms such as O and S between the carbon-carbon bonds forming the skeleton. R^{P}, R^{Q} and R^{T} are each independently a substituted or unsubstituted, straight or branched chain alkyl group having a carbon number of 1 to 12; a substituted or unsubstituted alicyclic hydrocarbon group having a carbon number of 5 to 25; and a substituted or unsubstituted aromatic hydrocarbon group having a carbon number of 6 to 12. k1, k2 and k3 are each independently an integer of 0 to 5. When there are a plurality of R^{a1} to R^{a3} and a plurality of R^{P}, R^{Q} and R^{T}, a plurality of R^{a1} to R^{a3} and a plurality of R^{P}, R^{Q} and R^{T} may be each identical or different.

In the above formula (X-2), R^{b1} is a substituted or unsubstituted, straight chain or branched alkyl group or alkoxy group having a carbon number of 1 to 20; a substituted or unsubstituted acyl group having a carbon number of 2 to 8; or a substituted or unsubstituted aromatic hydrocarbon group having a carbon number of 6 to 8; or a hydroxy group. nₖ is 0 or 1. When nₖ is 0, k4 is an integer of 0 to 4. When nₖ is 1, k4 is an integer of 0 to 7. When there are a plurality of R^{b1}, a plurality of R^{b1} may be each identical or different. A plurality of R^{b1} may represent a ring structure obtained by combining them. R^{b2} is a substituted or unsubstituted, straight chain or branched alkyl group having a carbon number of 1 to 7; or a substituted or unsubstituted aromatic hydrocarbon group having a carbon number of 6 or 7. L^{C} is a single bond or divalent linking group. k5 is an integer of 0 to 4. When there are a plurality of R^{b2}, a plurality of R^{b2} may be each identical or different. A plurality of R^{b2} may represent a ring structure obtained by combining them. q is an integer of 0 to 3. In formula, the ring structure containing S⁺ may contain a heteroatom such as O or S between the carbon-carbon bonds forming the skeleton.

In the above formula (X-3), R^{c1}, R^{c2} and R^{c3} are each independently a substituted or unsubstituted, straight or branched chain alkyl group having a carbon number of 1 to 12.

In the above formula (X-4), R^{g1} is a substituted or unsubstituted linear or branched alkyl or alkoxy group having 1 to 20 carbon atoms, a substituted or unsubstituted acyl group having 2 to 8 carbon atoms, a substituted or unsubstituted aromatic hydrocarbon group having 6 to 8 carbon atoms, or a hydroxy group. nₖ is 0 or 1. When nₖ₂ is 0, k10 is an integer of 0 to 4, and when nₖ₂ is 1, k10 is an integer of 0 to 7. When there are two or more R^{g1}s, the two or more R^{g1}s are the same or different from each other, and may represent a cyclic structure formed by combining them together. R^{g2} and R^{g3} are each independently a substituted or unsubstituted linear or branched alkyl, alkoxy, or alkoxycarbonyloxy group having 1 to 12 carbon atoms, a substituted or unsubstituted monocyclic or polycyclic cycloalkyl group having 3 to 12 carbon atoms, a substituted or unsubstituted aromatic hydrocarbon group having 6 to 12 carbon atoms, a hydroxyl group, a halogen atom, or a ring structure formed by combining two or more of these groups together. K11 and k12 are each independently an integer of 0 to 4. When there are two or more R^{g2}s and two or more R^{g3}s, the two or more R^{g2}s may be the same or different from each other, and the two or more R^{g3}s may be the same or different from each other.

In the above formula (X-5), R^{d1} and R^{d2} are each independently a substituted or unsubstituted, straight or branched chain alkyl group, alkoxy group or alkoxycarbonyl group having a carbon number of 1 to 12; a substituted or unsubstituted aromatic hydrocarbon group having a carbon number of 6 to 12; a halogen atom; a halogenated alkyl group having a carbon number of 1 to 4; a nitro group; or a ring structure obtained by combining two or more of these groups. k6 and k7 are each independently an integer of 0 to 5. When there are a plurality of R^{d1} and a plurality of R^{d2}, a plurality of R^{d1} and a plurality of R^{d2} may be each identical or different.

In the above formula (X-6), R^{e1} and R^{e2} are each independently a halogen atom; a substituted or unsubstituted straight or branched chain alkyl group having a carbon number of 1 to 12; or a substituted or unsubstituted aromatic hydrocarbon group having a carbon number of 6 to 12. k8 and k9 are each independently an integer of 0 to 4.

Specific examples of the onium cation include, but not limited thereto, structures represented by formulas (i-2-1) to (i-2-44).

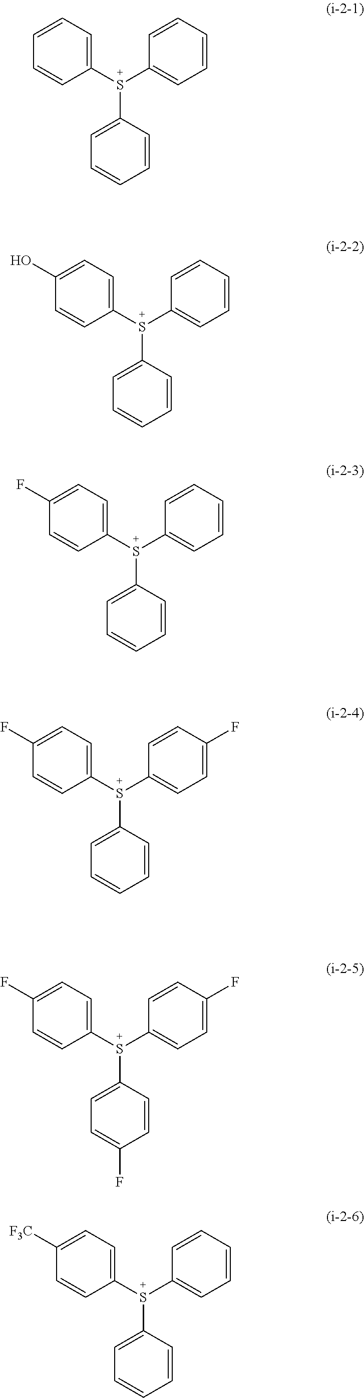

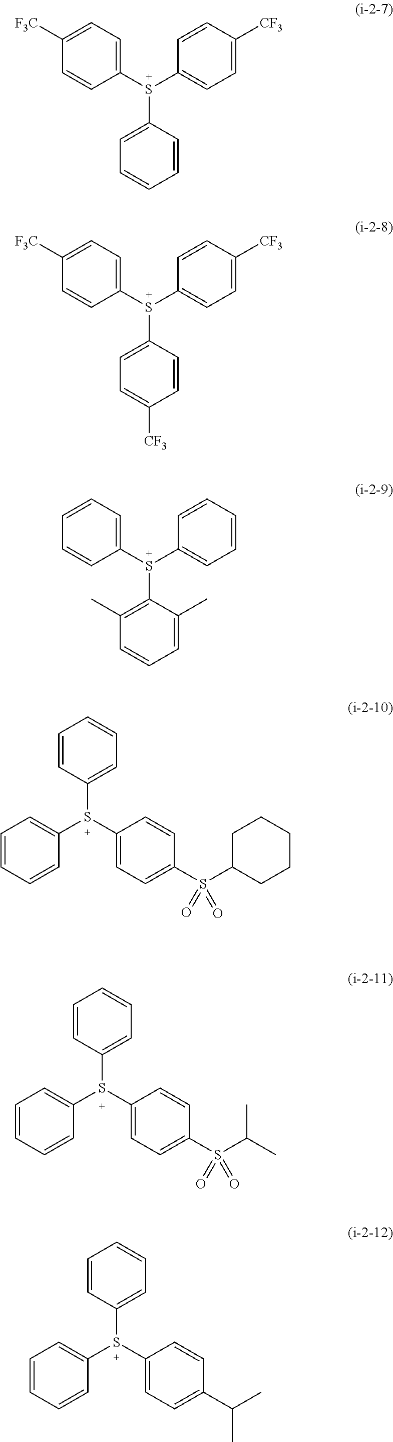

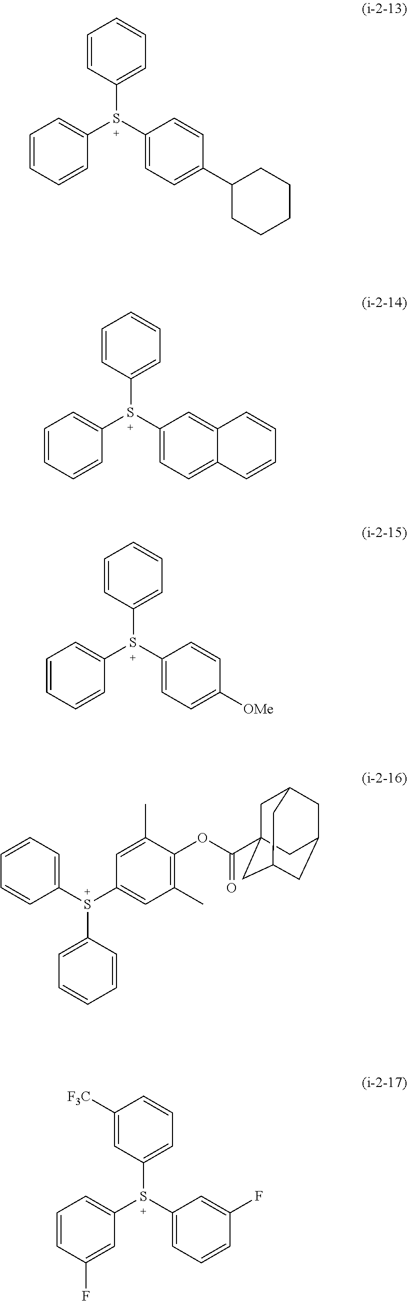

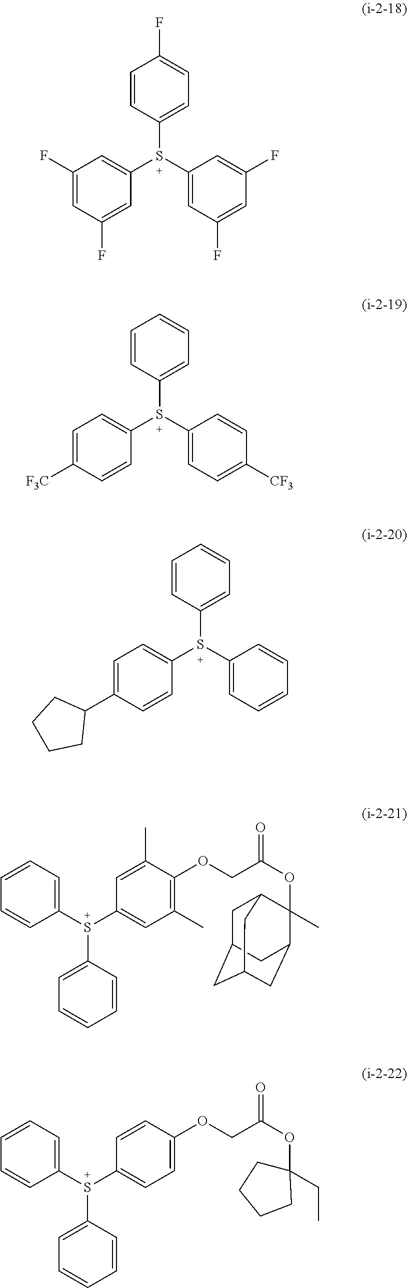

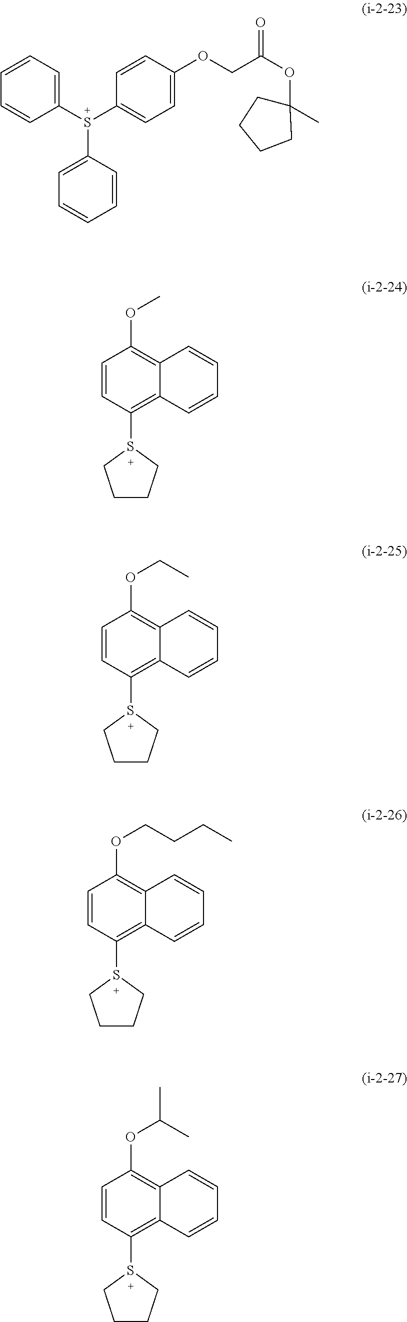

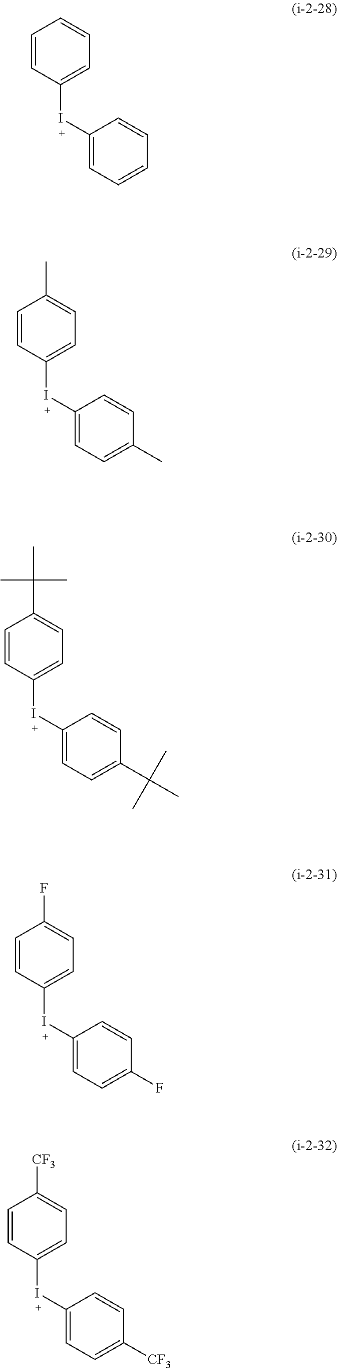

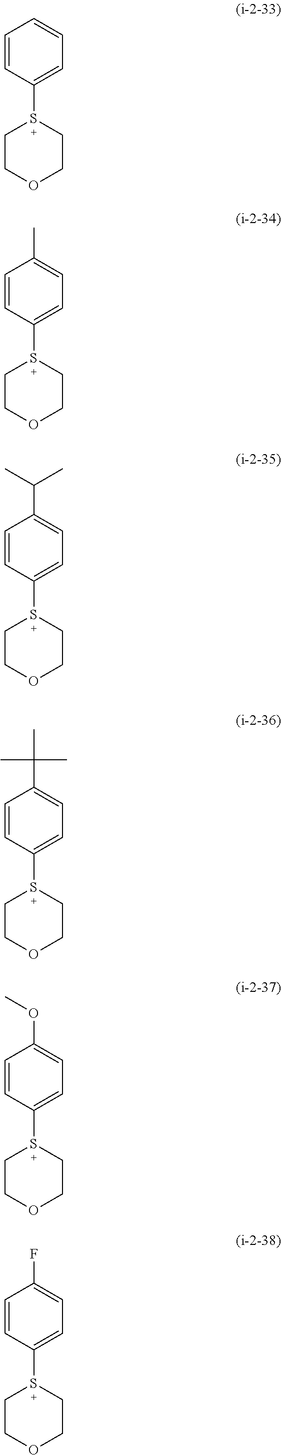

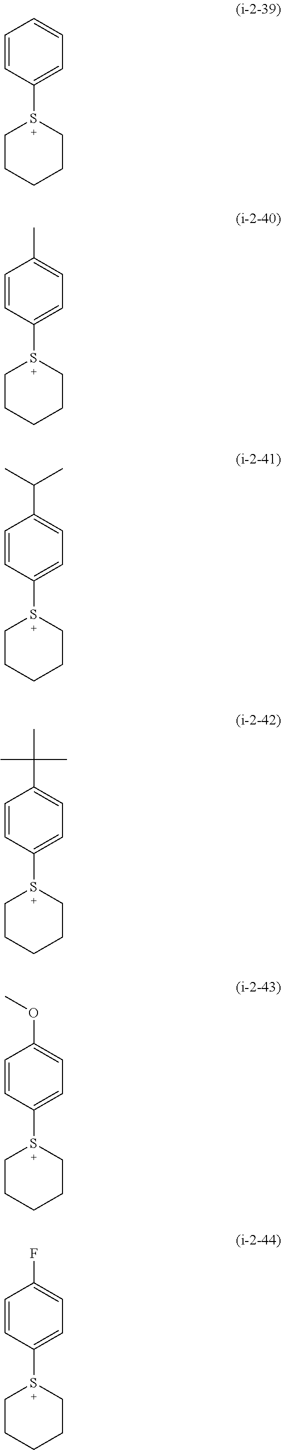

Examples of the onium salt (i) include structures obtained by any combination between the anion moieties and onium cations described above. Specific examples of the onium salt (i) include, but not limited thereto, onium salts represented by formulas (i-1) to (i-41).

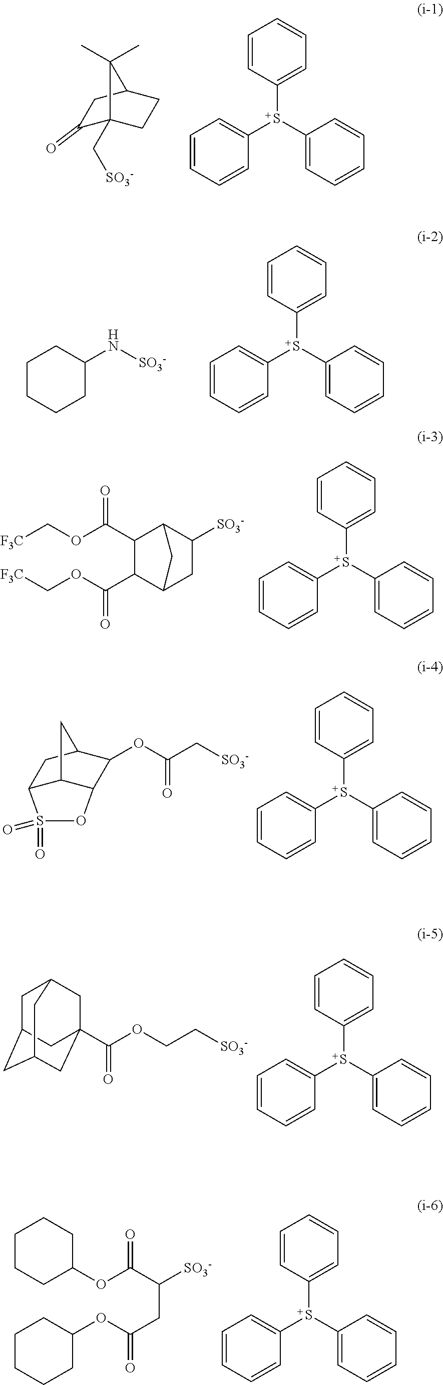

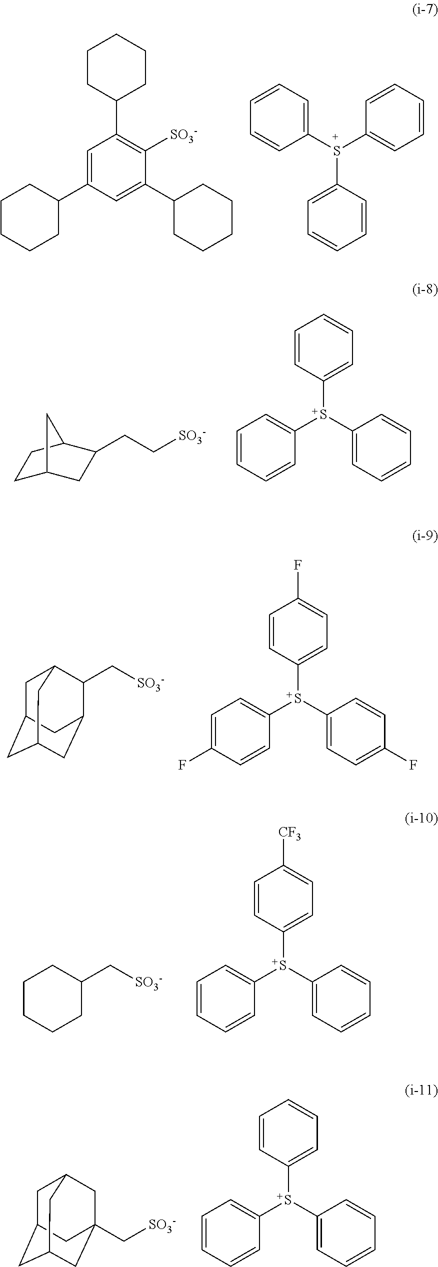

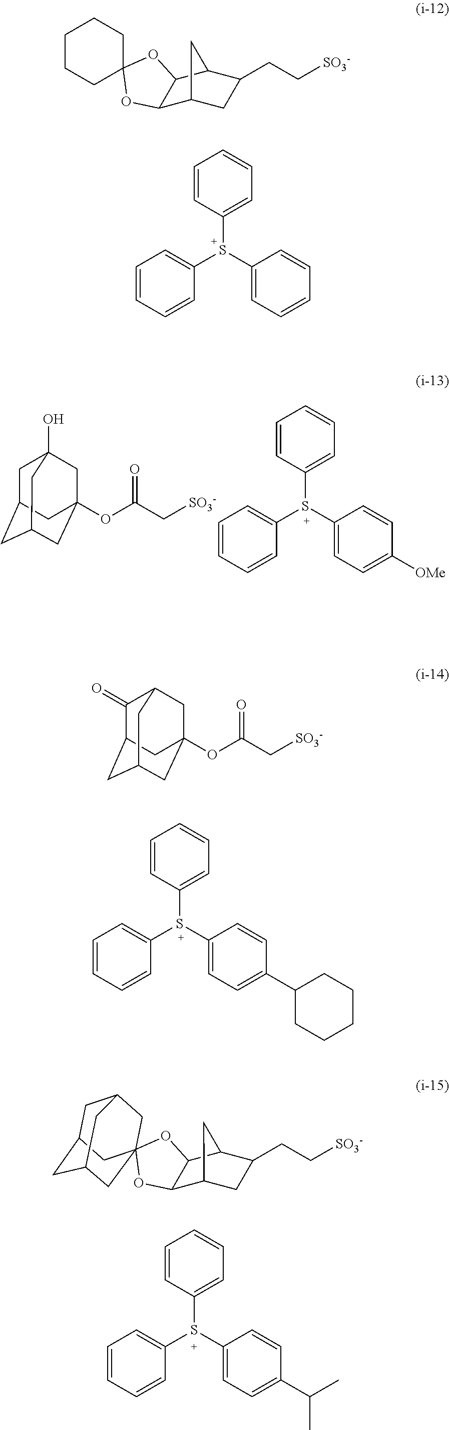

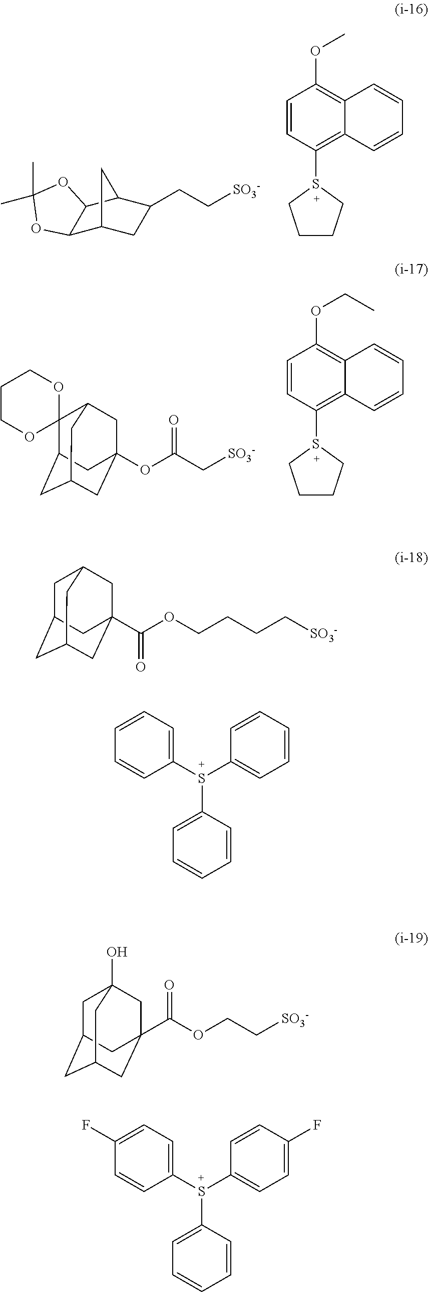

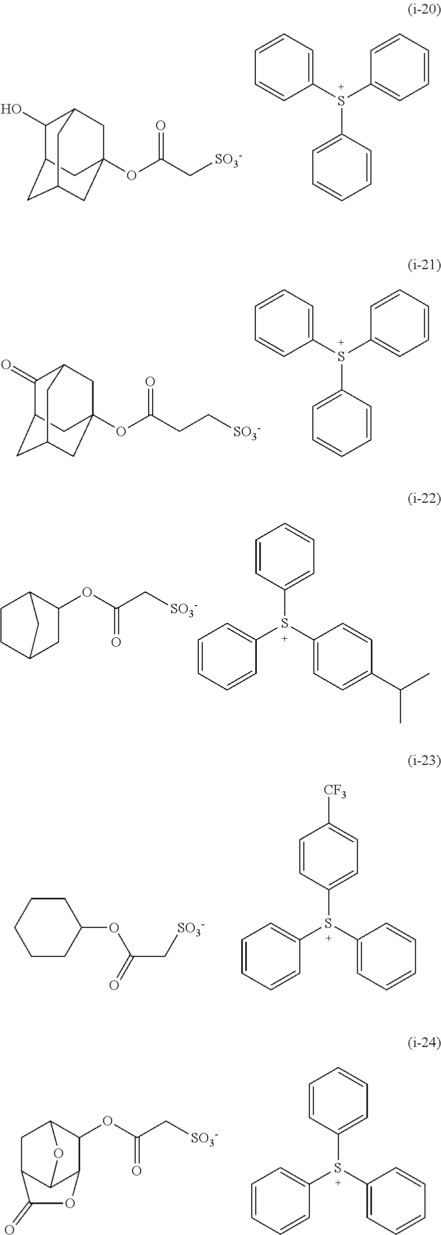

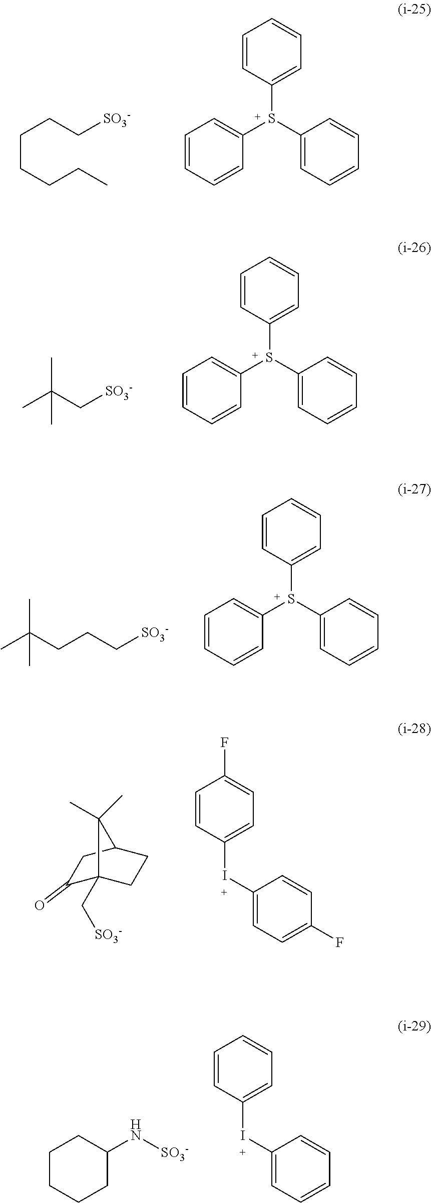

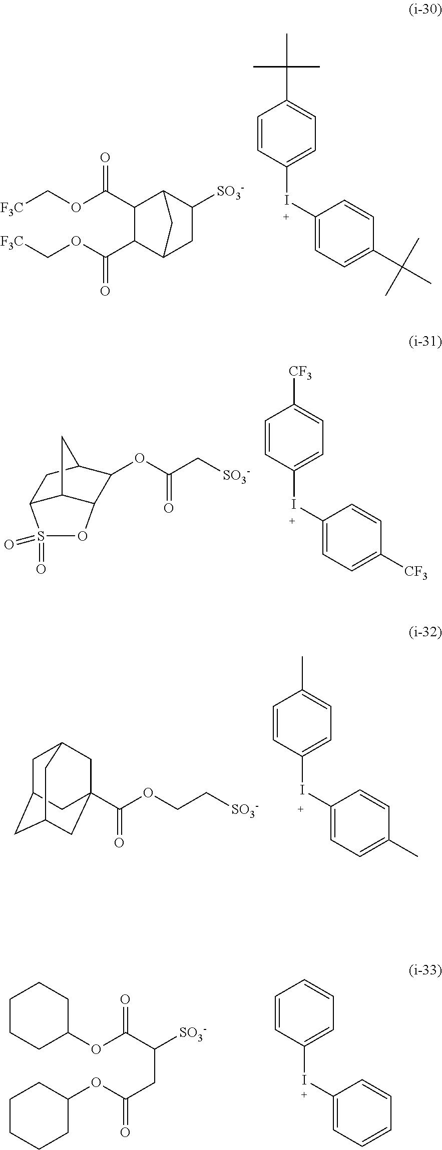

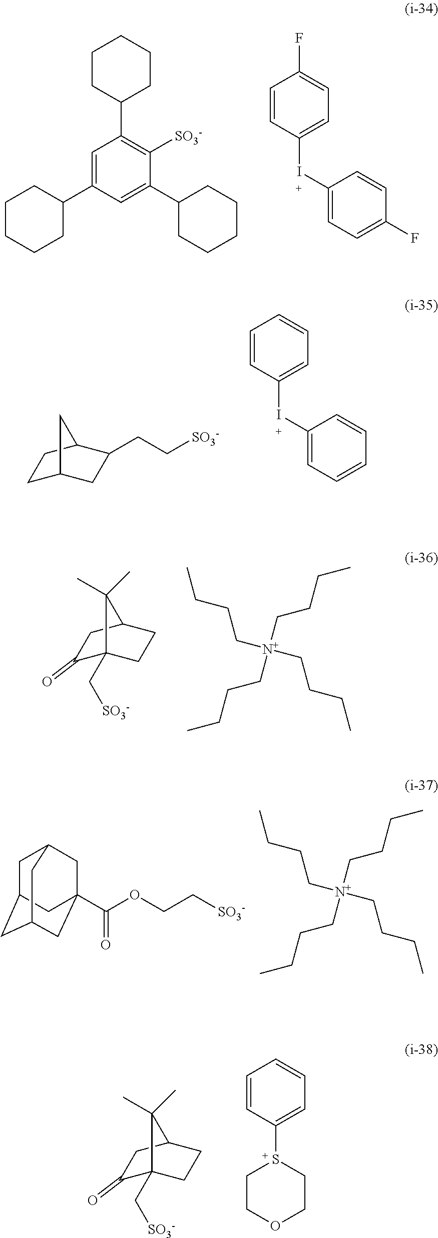

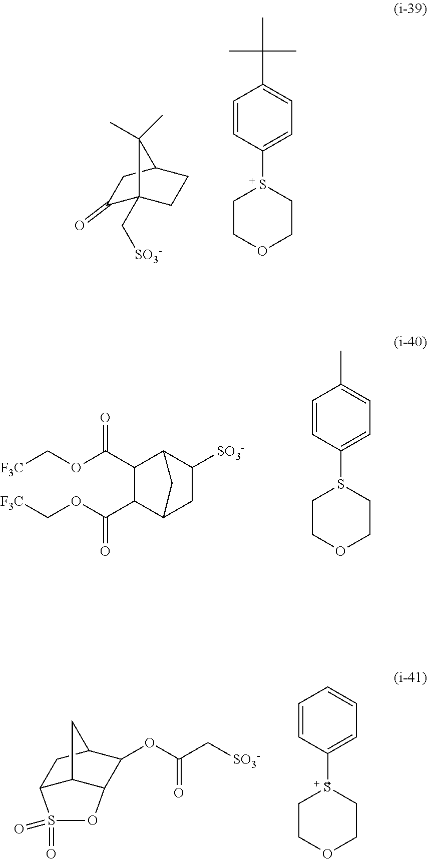

The lower limit of the content of the onium salt (i) (the total when a plurality of onium salts (i) are present) in the total mass of the components other than the solvent in the radiation-sensitive resin composition is preferably 0.1% by mass, more preferably 1% by mass, still more preferably 2% by mass, and particularly preferably 4% by mass. The upper limit of the content of the onium salt (i) is preferably 30% by mass, more preferably 25% by mass, still more preferably 20% by mass, and particularly preferably 15% by mass.

### <Radiation-Sensitive Acid Generator>

The radiation-sensitive acid generator is a component that generates an acid. When the polymer and the base resin contain the structural unit (III) having an acid-dissociable group, the acid generated from the radiation-sensitive acid generator by exposure has a function of dissociating the acid-dissociable group of the structural unit (III) and generating a carboxy group or the like.

When the onium salt (i) contains a radiation-sensitive onium cation, an acid is generated from the onium salt (i) by exposure. Under the conditions of pattern formation using the radiation-sensitive resin composition, this generated acid has a function of substantially preventing the acid-dissociable group of the structural unit (III) from being dissociated, and suppressing, in the unexposed part, the diffusion of the acid generated from the radiation-sensitive acid generator. That is, with or without radiation sensitivity, the onium salt (i) functions as an acid diffusion controlling agent.

The acid generated from the radiation-sensitive acid generator can be said to be relatively stronger (acid having a lower pKa) than the acid generated from the onium salt (i) having radiation sensitivity. The contained state of the radiation-sensitive acid generator in the radiation-sensitive resin composition may be a state in which the radiation-sensitive acid generator is present singly as a compound (released from the polymer), a state in which the radiation-sensitive acid generator is incorporated as a part of the polymer, or both of these states.

When the radiation-sensitive resin composition contains the radiation-sensitive acid generator, the polarity of the resin in the exposed part increases, whereby the resin in the exposed part is soluble in the developer in the case of alkaline aqueous solution development, and is poorly soluble in the developer in the case of organic solvent development.

Examples of the radiation-sensitive acid generator include an onium salt compound, a sulfonimide compound, a halogen-containing compound, and a diazoketone compound. Examples of the onium salt compound include a sulfonium salt, a tetrahydrothiophenium salt, an iodonium salt, a phosphonium salt, a diazonium salt, and a pyridinium salt. Among them, a sulfonium salt and an iodonium salt are preferable.

Examples of the acid generated by exposure include an acid that generates sulfonic acid, carboxylic acid, or sulfonimide by exposure. Examples of such an acid include a compound in which a carbon atom adjacent to a sulfo group is substituted with one or more fluorine atoms or a fluorinated hydrocarbon group.

These radiation-sensitive acid generators may be used singly or in combination of two or more thereof. From the viewpoint of securing the sensitivity and the developability as a resist, the lower limit of the content amount of the radiation-sensitive acid generator (the total when a plurality of radiation-sensitive acid generators are present) is preferably 2 parts by mass, more preferably 5 parts by mass, and still more preferably 8 parts by mass, with respect to 100 parts by mass of the base resin. From the viewpoint of securing the transparency to a radiation, the upper limit of the content amount of the radiation-sensitive acid generator is preferably 30 parts by mass, more preferably 25 parts by mass, and still more preferably 20 parts by mass, with respect to 100 parts by mass of the base resin.

### <Solvent>

The radiation-sensitive resin composition contains a solvent. The solvent is not particularly limited as long as the solvent is capable of dissolving or dispersing at least the polymer, and the base resin and the radiation-sensitive acid generator suitably contained, and the other additives and the like contained as desired.

Examples of the solvent include an alcohol-based solvent, an ether-based solvent, a ketone-based solvent, an amide-based solvent, an ester-based solvent, and a hydrocarbon-based solvent.

Examples of the alcohol-based solvent include:
a monoalcohol-based solvent having a carbon number of 1 to 18, including iso-propanol, 4-methyl-2-pentanol, 3-methoxybutanol, n-hexanol, 2-ethylhexanol, furfuryl alcohol, cyclohexanol, 3,3,5-trimethylcyclohexanol, and diacetone alcohol;
a polyhydric alcohol having a carbon number of 2 to 18, including ethylene glycol, 1,2-propylene glycol, 2-methyl-2,4-pentanediol, 2,5-hexanediol, diethylene glycol, dipropylene glycol, triethylene glycol, and tripropylene glycol; and a partially etherized polyhydric alcohol-based solvent in which a part of hydroxy groups in the polyhydric alcohol-based solvent is etherized.

Examples of the ether-based solvent include:
a dialkyl ether-based solvent, including diethyl ether, dipropyl ether, and dibutyl ether;
a cyclic ether-based solvent, including tetrahydrofuran and tetrahydropyran;
an ether-based solvent having an aromatic ring, including diphenylether and anisole (methyl phenyl ether); and
an etherized polyhydric alcohol-based solvent in which a hydroxy group in the polyhydric alcohol-based solvent is etherized.

Examples of the ketone-based solvent include:
a chain ketone-based solvent, including acetone, butanone, and methyl-iso-butyl ketone;
a cyclic ketone-based solvent, including cyclopentanone, cyclohexanone, and methylcyclohexanone; and
2,4-pentanedione, acetonylacetone, and acetophenone.

Examples of the amide-based solvent include:
a cyclic amide-based solvent, including N, N′-dimethyl imidazolidinone and N-methylpyrrolidone; and
a chain amide-based solvent, including N-methylformamide, N, N-dimethylformamide, N, N-diethylformamide, acetamide, N-methylacetamide, N, N-dimethylacetamide, and N-methylpropionamide.

Examples of the ester-based solvent include:
a monocarboxylate ester-based solvent, including n-butyl acetate and ethyl lactate;
a partially etherized polyhydric alcohol acetate-based solvent, including diethylene glycol mono-n-butyl ether acetate, propylene glycol monomethyl ether acetate, and dipropylene glycol monomethyl ether acetate;
a lactone-based solvent, including γ-butyrolactone and valerolactone;
a carbonate-based solvent, including diethyl carbonate, ethylene carbonate, and propylene carbonate; and
a polyhydric carboxylic acid diester-based solvent, including propylene glycol diacetate, methoxy triglycol acetate, diethyl oxalate, ethyl acetoacetate, ethyl lactate, and diethyl phthalate.

Examples of the hydrocarbon-based solvent include:
an aliphatic hydrocarbon-based solvent, including n-hexane, cyclohexane, and methylcyclohexane;
an aromatic hydrocarbon-based solvent, including benzene, toluene, di-iso-propylbenzene, and n-amylnaphthalene.

Among these examples, an ester-based solvent, an ether-based solvent, and a ketone-based solvent are preferable, a polyhydric alcohol partial ether acetate-based solvent, a polyhydric alcohol ether-based solvent, a polyvalent carboxylic acid diester-based solvent, a cyclic ketone-based solvent, and a lactone-based solvent are more preferable, and propylene glycol monomethyl ether acetate, propylene glycol monomethyl ether, ethyl lactate, cyclohexanone, and Y-butyrolactone are still more preferable. The radiation-sensitive resin composition may contain one or two or more solvents.

### <Other Optional Components>

The radiation-sensitive resin composition may contain other optional components other than the above-descried components. Examples of other optional components include a cross-linking agent, a localization enhancing agent, a surfactant, an alicyclic backbone-containing compound, and a sensitizer. These other optional components may be used singly or in combination of two or more of them.

### <Method for Preparing Radiation-Sensitive Resin Composition>

The radiation-sensitive resin composition can be prepared, for example, by mixing, at the prescribed ratio, the polymer with the base resin, the radiation-sensitive acid generator, the acid diffusion controlling agent, and the solvent as necessary. The radiation-sensitive resin composition is, after the mixing, preferably filtered through, for example, a filter having a pore size of approximately 0.05 μm to 0.2 μm. The solid content concentration of the radiation-sensitive resin composition is usually 0.1% by mass to 50% by mass, preferably 0.5% by mass to 30% by mass, and more preferably 18 by mass to 20% by mass.

### <Method for Forming Pattern>

A pattern forming method according to an embodiment of the present invention includes:
a step (1) of applying the radiation-sensitive resin composition directly or indirectly on a substrate to form a resist film (hereinafter, also referred to as a “resist film forming step”);
a step (2) of exposing the resist film (hereinafter, also referred to as an “exposure step”); and
a step (3) of developing the exposed resist film (hereinafter, also referred to as a “developing step”).

The method for forming a resist pattern uses the radiation-sensitive resin composition that is capable of forming a resist film having excellent sensitivity, LWR performance, water repellency, and properties of suppressing development defects, and that has good storage stability, and therefore a high-quality resist pattern can efficiently be formed. Hereinafter, each of the steps will be described.

### [Resist Film Forming Step]

In this step (the above mentioned step (1)), a resist film is formed with the radiation-sensitive resin composition. Examples of the substrate on which the resist film is formed include one traditionally known in the art, including a silicon wafer, silicon dioxide, and a wafer coated with aluminum. An organic or inorganic antireflection film may be formed on the substrate, as disclosed in JP-B-06-12452 and JP-A-59-93448. Examples of the applicating method include a rotary coating (spin coating), flow casting, and roll coating. After applicating, a prebake (PB) may be carried out in order to evaporate the solvent in the film, if needed. The temperature of PB is typically from 60° C. to 150° C., and preferably from 80° C. to 130° C. The duration of PB is typically from 5 seconds to 600 seconds, and preferably from 10 seconds to 300 seconds. The thickness of the resist film formed is preferably from 10 nm to 1,000 nm, and more preferably from 10 nm to 500 nm.

As for the receding contact angle of the resist film after pre-baking, 70° or more is preferred, 72° or more is more preferred and 74° or more is even more preferred. The method of measuring the receding contact angle is as described in the Examples.

When the immersion exposure is carried out, the formed resist film may have a protective film for the immersion which is not soluble into the immersion liquid on the film in order to prevent a direct contact between the immersion liquid and the resist film. As the protective film for the immersion, a solvent-removable protective film that is removed with a solvent before the developing step (for example, see JP-A-2006-227632); or a developer-removable protective film that is removed during the development of the developing step (for example, see WO2005-069076 and WO2006-035790) may be used. In terms of the throughput, the developer-removable protective film is preferably used.

When the exposing step that is the next step is performed with a radiation having a wavelength of 50 nm or less, it is preferable to use, as the base resin in the composition, a resin containing the structural units (III) and (VI), and the structural unit (V) as necessary.

### [Exposing Step]

In this step (the above mentioned step (2)), the resist film formed in the resist film forming step as the step (1) is exposed by irradiating with a radioactive ray through a photomask (optionally through an immersion medium such as water). Examples of the radioactive ray used for the exposure include visible ray, ultraviolet ray, far ultraviolet ray, extreme ultraviolet ray (EUV); an electromagnetic wave including X ray and γ ray; an electron beam; and a charged particle radiation such as α ray. Among them, far ultraviolet ray, an electron beam, or EUV is preferred. ArF excimer laser light (wavelength is 193 nm), KrF excimer laser light (wavelength is 248 nm), an electron beam, or EUV is more preferred. An electron beam or EUV having a wavelength of 50 nm or less which is identified as the next generation exposing technology is further preferred.

When the exposure is carried out by immersion exposure, examples of the immersion liquid include water and fluorine-based inert liquid. The immersion liquid is preferably a liquid which is transparent with respect to the exposing wavelength, and has a minimum temperature factor of the refractive index so that the distortion of the light image reflected on the film becomes minimum. However, when the exposing light source is ArF excimer laser light (wavelength is 193 nm), water is preferably used because of the ease of availability and ease of handling in addition to the above considerations. When water is used, a small proportion of an additive that decreases the surface tension of water and increases the surface activity may be added. Preferably, the additive cannot dissolve the resist film on the wafer and can neglect an influence on an optical coating at an under surface of a lens. The water used is preferably distilled water.

After the exposure, post exposure bake (PEB) is preferably carried out to promote the dissociation of the acid-dissociable group in the resin by the acid generated from the radiation-sensitive acid generator with the exposure in the exposed part of the resist film. The difference of solubility into the developer between the exposed part and the non-exposed part is generated by the PEB. The temperature of PEB is typically from 50° C. to 180° C., and preferably from 80° C. to 130° C. The duration of PEB is typically from 5 seconds to 600 seconds, and preferably from 10 seconds to 300 seconds.

### [Developing Step]

In this step (the above mentioned step (3)), the resist film exposed in the exposing step as the step (2) is developed. By this step, the predetermined resist pattern can be formed. After the development, the resist pattern is washed with a rinse solution such as water or alcohol, and the dried, in general.

Examples of the developer used for the development include, in the alkaline development, an alkaline aqueous solution obtained by dissolving at least one alkaline compound such as sodium hydroxide, potassium hydroxide, sodium carbonate, sodium silicate, sodium metasilicate, ammonia water, ethylamine, n-propylamine, diethylamine, di-n-propylamine, triethylamine, methyldiethylamine, ethyldimethylamine, triethanolamine, tetramethyl ammonium hydroxide (TMAH), pyrrole, piperidine, choline, 1,8-diazabicyclo-[5.4.0]-7-undecene, 1,5-diazabicyclo-[4.3.0]-5-nonene. Among them, an aqueous TMAH solution is preferred, and 2.38% by mass of aqueous TMAH solution is more preferred.

In the case of the development with organic solvent, examples of the solvent include an organic solvent, including a hydrocarbon-based solvent, an ether-based solvent, an ester-based solvent, a ketone-based solvent, and an alcohol-based solvent; and a solvent containing an organic solvent. Examples of the organic solvent include one, two or more solvents listed as the solvent for the radiation-sensitive resin composition. Among them, an ether-based solvent, an ester-based solvent or a ketone-based solvent is preferred. As the ether-based solvent, a glycol ether-based solvent is preferable, and ethylene glycol monomethyl ether and propylene glycol monomethyl ether are more preferable. The ester-based solvent is preferably an acetate ester-based solvent, and more preferably n-butyl acetate or amyl acetate. The ketone-based solvent is preferably a chain ketone, and more preferably 2-heptanone. The content of the organic solvent in the developer is preferably not less than 80% by mass, more preferably not less than 90% by mass, further preferably not less than 95% by mass, and particularly preferably not less than 99% by mass. Examples of the ingredient other than the organic solvent in the developer include water and silicone oil.

As described above, the developer may be either an alkaline developer or an organic solvent developer, but it is preferable that the developer contains an alkaline aqueous solution and the obtained pattern is a positive pattern.

Examples of the developing method include a method of dipping the substrate in a tank filled with the developer for a given time (dip method); a method of developing by putting and leaving the developer on the surface of the substrate with the surface tension for a given time (paddle method); a method of spraying the developer on the surface of the substrate (spray method); and a method of injecting the developer while scanning an injection nozzle for the developer at a constant rate on the substrate rolling at a constant rate (dynamic dispense method).

### EXAMPLES

Hereinafter, the present disclosure will specifically be described with reference to examples, but the present invention is not limited to these examples. The methods for measuring various physical property values are described below.

### [Weight-Average Molecular Weight (Mw) and Number-Average Molecular Weight (Mn)]

The Mw and the Mn of polymers were measured by gel permeation chromatography (GPC) using GPC columns of Tosoh Corporation (“G2000HXL” x 2, “G3000HXL” x 1, “G4000HXL” x 1) under the following conditions. The degree of dispersion (Mw/Mn) was calculated from the measurement results of Mw and Mn.
Elution solvent: tetrahydrofuran
Flow rate: 1.0 mL/min
Sample concentration: 1.0% by mass
Amount of sample injected: 100 μL
Column temperature: 40° C.
Detector: differential refractometer
Standard substance: monodisperse polystyrene

### [¹³C-NMR Analysis]

The ¹³C-NMR analysis of polymers was performed using a nuclear magnetic resonance apparatus (“JNM-Delta 400” of JEOL

### Ltd.).

### Synthesis of Compound (Monomer) [F]

### Synthesis Example 1

### Synthesis of Compound (F-1)

A compound (F-1) (hereinafter, also referred to as a “monomer (F-1)”) was synthesized according to the following synthesis scheme.

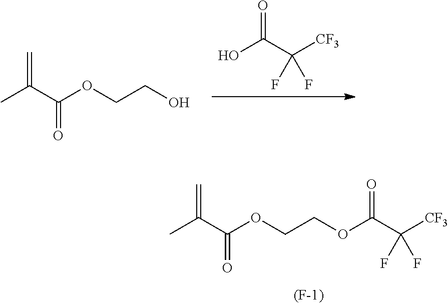

In a reaction container, 20 mmol of 2-hydroxyethyl methacrylate, 20.0 mmol of 1-(3-dimethylaminopropyl)-3-ethylcarbodiimide hydrochloride, 4.0 mmol of 1-dimethylaminopyridine, and 50 g of dichloromethane were mixed and cooled to 0° C. to give a solution. Into this solution, 20.0 mmol of pentafluoropropionic acid was delivered by drops and stirred for 1 hour. Thereafter, the solution was diluted by adding water, and dichloromethane was then added thereto and extracted therefrom to separate an organic layer. The organic layer obtained was washed with an aqueous solution of saturated sodium chloride and then with water. After the organic layer was dried over sodium sulfate, the solvent was distilled off, and the residue was purified by column chromatography, affording a compound (F-1) in a good yield.

### [Synthesis Examples 2 to 6] (Synthesis of Compounds (F-2) to (F-6))

Compounds represented by formulas (F-2) to (F-6) were synthesized in the same manner as in Synthesis Example 1 except that the raw materials and the precursor were changed as appropriate. (Hereinafter, the compounds represented by the formulas (F-2) to (F-6) are sometimes described as “compounds (F-2) to (F-6)” or “monomers (F-2) to (F-6)”. Hereinafter, the compound (F-1) is described together.)

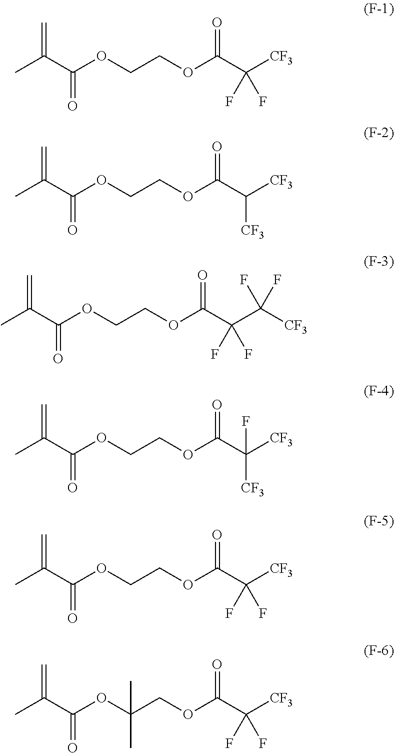

Among the monomers used in the synthesis of polymers and base resins, monomers other than the monomers (F-1) to (F-6) are described below. In the following synthesis examples, unless otherwise specified, a value represented by parts by mass is based on the total mass of the used monomers defined as 100 parts by mass, and a value represented by mol % is based on the total number of moles of the used monomers defined as 100 mol.

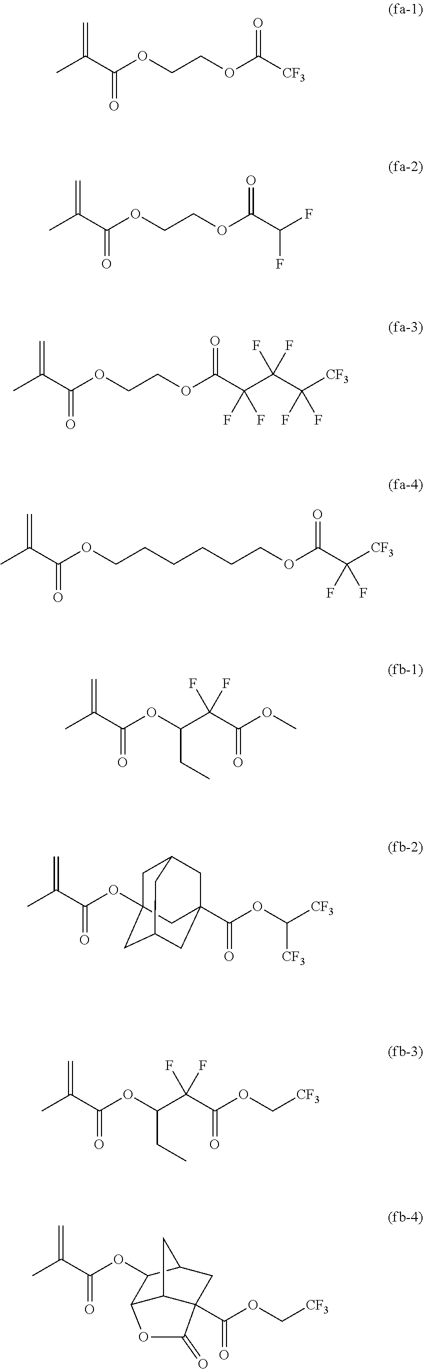

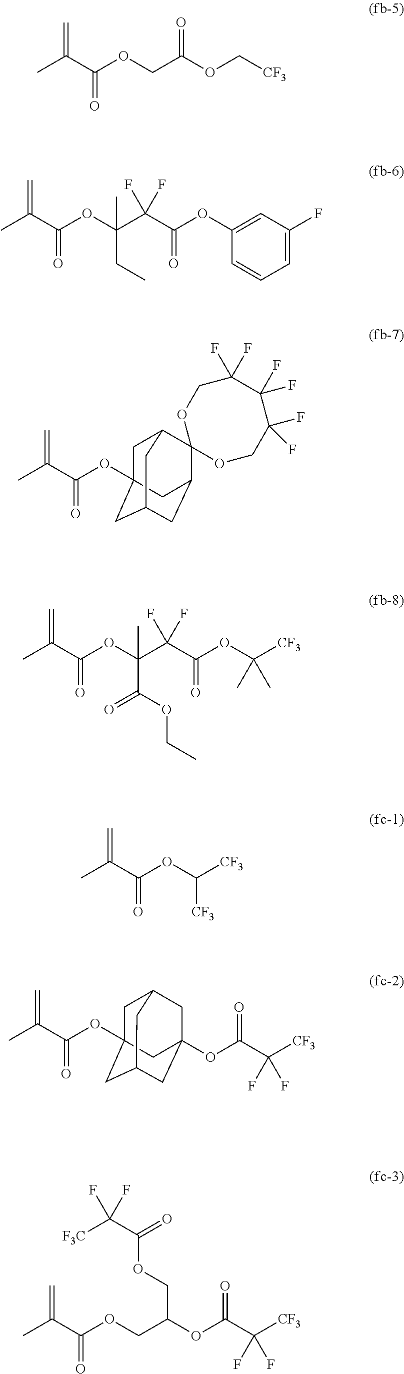

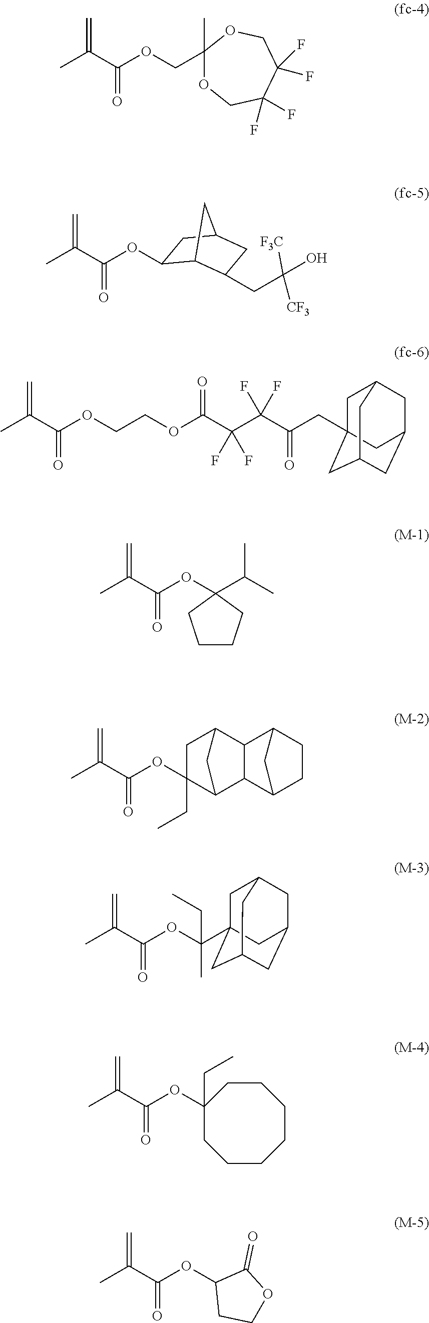

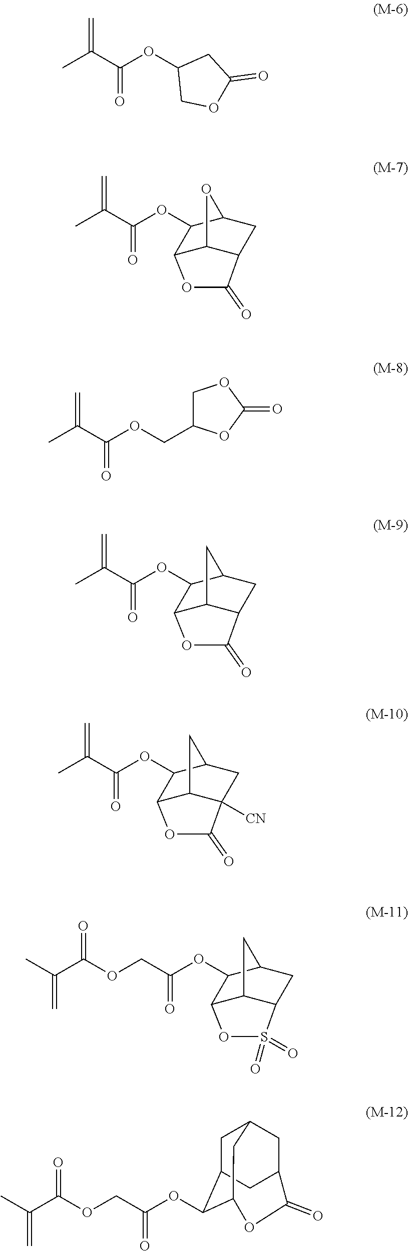

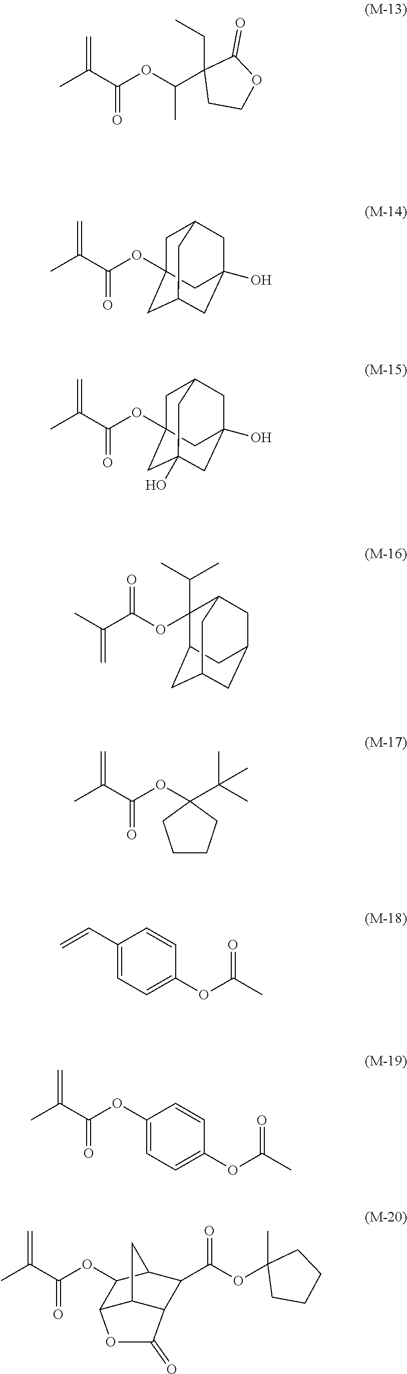

### Synthesis Example 7

### Synthesis of Base Resin (A-1)

The monomer (M-1), the monomer (M-2), and the monomer (M-10) were dissolved in 2-butanone (200 parts by mass) so as to have a molar ratio of 35/20/45 (mol %), and AIBN (azobisisobutyronitrile) (5 mol % with respect to 100 mol % in total of the used monomers) was added as an initiator to prepare a monomer solution. A reaction vessel was charged with 2-butanone (100 parts by mass) and purged with nitrogen for 30 minutes, and inside of the reaction vessel was adjusted to 80° C. Then, the monomer solution was added dropwise thereto over 3 hours with stirring. The polymerization reaction was performed for 6 hours with the start of dropwise addition as the initiation time of the polymerization reaction. After completion of the polymerization reaction, the polymerization solution was cooled to 30° C. or lower by water cooling. The cooled polymerization solution was added to methanol (2,000 parts by mass), and the precipitated white powder was separated by filtration. The separated white powder was washed with methanol twice, then separated by filtration, and dried at 50° C. for 24 hours to obtain a white powdery base resin (A-1) (yield: 74%). The resin (A-1) had a Mw of 5,800 and a Mw/Mn of 1.53. As a result of ¹³C-NMR analysis, the contents of the structural units derived from (M-1), (M-2), and (M-10) were 34.8 mol %, 19.4 mol %, and 45.8 mol %, respectively.

### Synthesis Examples 8 to 17

### Synthesis of base resins (A-2) to (A-11)

Base resins (A-2) to (A-11) were synthesized in the same manner as in Synthesis Example 7 except that the types of monomers and the blending proportion shown in Table 1 below were used. Table 1 also shows the content (mol %) of each of the structural units and the physical property values (Mw and Mw/Min) of the base resins obtained. In Table 1, “-” indicates that the corresponding monomer was not used (the same applies to the following tables).

TABLE 1

| | | Monomer that gives | | | Monomer that gives | | | Monomer that gives | | | | |
|---|---|---|---|---|---|---|---|---|---|---|---|---|
| | | structural unit (III) | | | structural unit (IV) | | | structural unit (V) | | | | |
| | Base | | Blending | Content of | | Blending | Content of | | Blending | Content of | | |
| | resin | | proportion | structural | | proportion | structural | | proportion | structural | | |
| | [A] | Type | (mol %) | unit (mol %) | Type | (mol %) | unit (mol %) | Type | (mol %) | unit (mol %) | Mw | Mw/Mn |
| Synthesis Example 7 | A-1 | M-1 | 35 | 34.8 | M-10 | 45 | 45.8 | — | — | — | 5800 | 1.53 |
| Synthesis Example 8 | A-2 | M-1 | 30 | 29.8 | M-9 | 40 | 43.0 | — | — | — | 6300 | 1.58 |
| Synthesis Example 9 | A-3 | M-1 | 25 | 25.0 | M-11 | 60 | 59.9 | — | — | — | 7000 | 1.58 |
| Synthesis Example 10 | A-4 | M-1 | 35 | 34.8 | M-8 | 45 | 45.6 | — | — | — | 6600 | 1.52 |
| Synthesis Example 11 | A-5 | M-1 | 45 | 46.0 | M-5 | 45 | 44.9 | — | — | — | 5900 | 1.56 |
| Synthesis Example 12 | A-6 | M-1 | 40 | 39.2 | M-7 | 45 | 48.3 | — | — | — | 7600 | 1.67 |
| Synthesis Example 13 | A-7 | M-1 | 40 | 39.7 | M-12 | 45 | 46.1 | M-14 | 15 | 14.2 | 5400 | 1.53 |
| Synthesis Example 14 | A-8 | M-1 | 40 | 39.5 | M-6 | 45 | 47.4 | M-15 | 15 | 13.1 | 5300 | 1.52 |
| Synthesis Example 15 | A-9 | M-1 | 30 | 31.1 | M-5 | 20 | 21.7 | M-14 | 10 | 8.1 | 5200 | 1.52 |
| Synthesis Example 16 | A-10 | M-1 | 30 | 31.5 | M-6 | 20 | 21.1 | M-15 | 10 | 9.6 | 6500 | 1.59 |
| Synthesis Example 17 | A-11 | M-16 | 40 | 38.7 | M-5 | 30 | 31.2 | — | — | — | 6100 | 1.55 |

### Synthesis Example 12

### Synthesis of base resin (A-12)

The monomer (M-1) and the monomer (M-18) were dissolved in 1-methoxy-2-propanol (200 parts by mass) so as to have a molar ratio of 50/50 (mol %), and AIBN (5 mol %) was added as an initiator to prepare a monomer solution. A reaction vessel was charged with 1-methoxy-2-propanol (100 parts by mass) and purged with nitrogen for 30 minutes, and inside of the reaction vessel was adjusted to 80° C. Then, the monomer solution was added dropwise thereto over 3 hours with stirring. The polymerization reaction was performed for 6 hours with the start of dropwise addition as the initiation time of the polymerization reaction. After completion of the polymerization reaction, the polymerization solution was cooled to 30° C. or lower by water cooling. The cooled polymerization solution was added to hexane (2,000 parts by mass), and the precipitated white powder was separated by filtration. The separated white powder was washed twice with hexane, then separated by filtration, and dissolved in 1-methoxy-2-propanol (300 parts by mass). Subsequently, methanol (500 parts by mass), triethylamine (50 parts by mass), and ultrapure water (10 parts by mass) were added, and a hydrolysis reaction was performed at 70° C. for 6 hours with stirring. After completion of the reaction, the remaining solvent was distilled off, and the obtained solid was dissolved in acetone (100 parts by mass). The solution was added dropwise to water (500 parts by mass) to solidify the resin. The resulting solid was separated by filtration and dried at 50° C. for 13 hours to obtain a white powdery base resin (A-12) (yield: 75%). The base resin (A-12) had a Mw of 6,100 and a Mw/Mn of 1.49. As a result of ¹³C-NMR analysis, the contents of the structural units derived from (M-1) and (M-18) were 49.2 mol % and 50.8 mol %, respectively.

### Synthesis Examples 19 to 21

### Synthesis of Base Resins (A-13) to (A-15)

Base resins (A-13) to (A-15) were synthesized in the same manner as in Synthesis Example 18 except that the types of monomers and the blending proportion shown in Table 2 below were used. Table 2 also shows the content (mol %) of each of the structural units and the physical property values (Mw and Mw/Mn) of the resins obtained.

TABLE 2

| | | Monomer that gives | | | Monomer that gives | | | Monomer that gives | | | | |
|---|---|---|---|---|---|---|---|---|---|---|---|---|
| | | structural unit (III) | | | structural unit (V) | | | structural unit (IV) | | | | |
| | | | | Content of | | | Content of | | | Content of | | |
| | Base | | Blending | structural | | Blending | structural | | Blending | structural | | |
| | resin | | proportion | unit | | proportion | unit | | proportion | unit | | |
| | [A] | Type | (mol %) | (mol %) | Type | (mol %) | (mol %) | Type | (mol %) | (mol %) | Mw | Mw/Mn |
| Synthesis | A-12 | M-1 | 50 | 49.2 | — | — | — | M-18 | 50 | 50.8 | 6100 | 1.49 |
| Example 18 | | | | | | | | | | | | |
| Synthesis | A-13 | M-3 | 40 | 39.4 | M-14 | 20 | 19.0 | M-19 | 40 | 41.6 | 6500 | 1.55 |
| Example 19 | | | | | | | | | | | | |
| Synthesis | A-14 | M-2 | 45 | 43.6 | M-15 | 10 | 13.2 | M-18 | 45 | 43.2 | 6500 | 1.54 |
| Example 20 | | | | | | | | | | | | |
| Synthesis | A-15 | M-16 | 50 | 47.2 | M-14 | 25 | 25.1 | M-19 | 25 | 27.7 | 6400 | 1.51 |
| Example 21 | | | | | | | | | | | | |

### Synthesis Example 22

### Synthesis of Polymer (E-1)

The monomer (F-1), monomer (fb-1) and the monomer (M-1) were dissolved in 2-butanone (200 parts by mass) so as to have a molar ratio of 35/30/35 (mol %), and AIBN (3 mol %) was added as an initiator to prepare a monomer solution. A reaction vessel was charged with 2-butanone (100 parts by mass) and purged with nitrogen for 30 minutes, and inside of the reaction vessel was adjusted to 80° C. Then, the monomer solution was added dropwise thereto over 3 hours with stirring. The polymerization reaction was performed for 6 hours with the start of dropwise addition as the initiation time of the polymerization reaction. After completion of the polymerization reaction, the polymerization solution was cooled to 30° C. or lower by water cooling. The operation of replacing the solvent with acetonitrile (400 parts by mass), then adding hexane (100 parts by mass), stirring the mixture, and recovering the acetonitrile layer was repeated three times. The solvent was replaced with propylene glycol monomethyl ether acetate to obtain a solution of a polymer (E-1) (yield: 75%). The polymer (E-1) had a Mw of 9,200 and a Mw/Mn of 1.51. As a result of ¹³C-NMR analysis, the contents of the structural units derived from (F-1), (fb-1) and (M-1) were 34.4 mol %, 31.2 mol % and 34.4 mol %, respectively.

### Synthesis Examples 23 to 60

### Synthesis of polymers (E-2) to (E-34) and polymers (CE-1) to (CE-5)

Polymers (E-2) to (E-34) and polymers (CE-1) to (CE-5) were synthesized in the same manner as in Synthesis Example 22 except that the types of monomers and the blending proportion shown in Tables 3 and 4 below were used. Tables 3 and 4 also show the content (mol %) of each of the structural units and the physical property values (Mw and Mw/Mn) of the polymers obtained.

TABLE 3

| | | Monomer that gives | | | Monomer that gives | | | Monomers that give structural | | | | |
|---|---|---|---|---|---|---|---|---|---|---|---|---|
| | | structural unit (I) | | | structural unit (II) | | | units (III) to (V) | | | | |
| | | | | Content of | | | Content of | | | Content of | | |
| | | | Blending | structural | | Blending | structural | | Blending | structural | | |
| | | | proportion | unit | | proportion | unit | | proportion | unit | | |
| | Polymer [E] | Type | (mol %) | (mol %) | Type | (mol %) | (mol %) | Type | (mol %) | (mol %) | Mw | Mw/Mn |
| Synthesis | E-1 | F-1 | 35 | 34.4 | fb-1 | 30 | 31.2 | M-1 | 35 | 34.4 | 9200 | 1.51 |
| Example 22 | | | | | | | | | | | | |
| Synthesis | E-2 | F-1 | 35 | 35.3 | fb-2 | 35 | 34.4 | M-1 | 30 | 30.3 | 9800 | 1.55 |
| Example 23 | | | | | | | | | | | | |
| Synthesis | E-3 | F-1 | 35 | 35.5 | fb-3 | 30 | 29.1 | M-1 | 35 | 35.4 | 8900 | 1.49 |
| Example 24 | | | | | | | | | | | | |
| Synthesis | E-4 | F-1 | 40 | 40.8 | fb-4 | 45 | 45.0 | M-1 | 15 | 14.2 | 9200 | 1.48 |
| Example 25 | | | | | | | | | | | | |
| Synthesis | E-5 | F-1 | 30 | 30.6 | fb-5 | 20 | 20.8 | M-1 | 50 | 48.6 | 9100 | 1.46 |
| Example 26 | | | | | | | | | | | | |
| Synthesis | E-6 | F-1 | 45 | 44.8 | fb-6 | 45 | 45.4 | M-1 | 10 | 9.8 | 9000 | 1.52 |
| Example 27 | | | | | | | | | | | | |
| Synthesis | E-7 | F-1 | 35 | 34.9 | fb-7 | 35 | 35.2 | M-1 | 30 | 29.9 | 8400 | 1.51 |
| Example 28 | | | | | | | | | | | | |
| Synthesis | E-8 | F-1 | 20 | 20.1 | fb-8 | 20 | 19.8 | M-1 | 60 | 60.1 | 8100 | 1.50 |
| Example 29 | | | | | | | | | | | | |
| Synthesis | E-9 | F-1 | 30 | 30.1 | fc-1 | 40 | 40.5 | M-1 | 30 | 29.4 | 8900 | 1.58 |
| Example 30 | | | | | | | | | | | | |
| Synthesis | E-10 | F-1 | 30 | 29.9 | fc-2 | 40 | 39.9 | M-1 | 30 | 30.2 | 9100 | 1.43 |
| Example 31 | | | | | | | | | | | | |
| Synthesis | E-11 | F-1 | 30 | 30.4 | fc-3 | 20 | 19.8 | M-1 | 50 | 49.8 | 9000 | 1.49 |
| Example 32 | | | | | | | | | | | | |
| Synthesis | E-12 | F-1 | 30 | 30.0 | fc-4 | 40 | 39.6 | M-1 | 30 | 30.4 | 8700 | 1.55 |
| Example 33 | | | | | | | | | | | | |
| Synthesis | E-13 | F-1 | 30 | 31.1 | fc-5 | 30 | 30.3 | M-1 | 40 | 38.6 | 9200 | 1.49 |
| Example 34 | | | | | | | | | | | | |
| Synthesis | E-14 | F-1 | 30 | 30.3 | fc-6 | 30 | 29.8 | M-1 | 40 | 39.9 | 8200 | 1.51 |
| Example 35 | | | | | | | | | | | | |
| Synthesis | E-15 | F-2 | 35 | 34.9 | fb-1 | 30 | 30.2 | M-1 | 35 | 34.9 | 8100 | 1.60 |
| Example 36 | | | | | | | | | | | | |
| Synthesis | E-16 | F-3 | 35 | 35.1 | fb-1 | 30 | 29.8 | M-1 | 35 | 35.1 | 9200 | 1.43 |
| Example 37 | | | | | | | | | | | | |
| Synthesis | E-17 | F-4 | 35 | 35.6 | fb-1 | 30 | 28.8 | M-1 | 35 | 35.6 | 8000 | 1.52 |
| Example 38 | | | | | | | | | | | | |
| Synthesis | E-18 | F-5 | 35 | 34.6 | fb-1 | 30 | 30.8 | M-1 | 35 | 34.6 | 8500 | 1.55 |
| Example 39 | | | | | | | | | | | | |
| Synthesis | E-19 | F-6 | 35 | 35.2 | fb-1 | 30 | 29.6 | M-1 | 35 | 35.2 | 8200 | 1.49 |
| Example 40 | | | | | | | | | | | | |
| Synthesis | E-20 | F-1 | 45 | 45.2 | fb-1 | 40 | 39.6 | M-2 | 15 | 15.2 | 9100 | 1.48 |
| Example 41 | | | | | | | | | | | | |
| Synthesis | E-21 | F-1 | 45 | 45.4 | fb-1 | 40 | 40.1 | M-3 | 15 | 14.5 | 9200 | 1.60 |
| Example 42 | | | | | | | | | | | | |
| Synthesis | E-22 | F-1 | 20 | 20.6 | fb-1 | 40 | 39.6 | M-4 | 40 | 39.8 | 8800 | 1.46 |
| Example 43 | | | | | | | | | | | | |
| Synthesis | E-23 | F-1 | 35 | 34.4 | fb-1 | 40 | 40.1 | M-6 | 25 | 25.5 | 9300 | 1.59 |
| Example 44 | | | | | | | | | | | | |
| Synthesis | E-24 | F-1 | 35 | 35.3 | fb-1 | 30 | 29.8 | M-7 | 35 | 34.9 | 9100 | 1.71 |
| Example 45 | | | | | | | | | | | | |
| Synthesis | E-25 | F-1 | 35 | 35.5 | fb-1 | 30 | 28.8 | M-10 | 35 | 35.7 | 8800 | 1.58 |
| Example 46 | | | | | | | | | | | | |
| Synthesis | E-26 | F-1 | 40 | 40.8 | fb-1 | 30 | 30.8 | M-14 | 30 | 28.4 | 8700 | 1.54 |
| Example 47 | | | | | | | | | | | | |
| Synthesis | E-27 | F-1 | 30 | 30.6 | fb-1 | 30 | 29.6 | M-15 | 40 | 39.8 | 8100 | 1.60 |
| Example 48 | | | | | | | | | | | | |
| Synthesis | E-28 | F-1 | 45 | 45.2 | fb-1 | 30 | 28.8 | M-16 | 25 | 26.0 | 8300 | 1.55 |
| Example 49 | | | | | | | | | | | | |
| Synthesis | E-29 | F-1 | 40 | 41.3 | fb-1 | 40 | 39.5 | M-17 | 20 | 19.2 | 8500 | 1.61 |
| Example 50 | | | | | | | | | | | | |
| Synthesis | E-30 | F-1 | 45 | 44.5 | fb-1 | 40 | 40.3 | M-20 | 15 | 15.2 | 9500 | 1.47 |
| Example 51 | | | | | | | | | | | | |
| Synthesis | E-31 | F-1 | 40 | 41.2 | fb-4 | 20 | 19.3 | M-11 | 10 | 10.0 | 9100 | 1.59 |
| Example 52 | | | | | fc-1 | 20 | 20.0 | M-17 | 10 | 9.5 | | |
| Synthesis | E-32 | F-1 | 40 | 42.4 | fb-5 | 20 | 20.2 | M-12 | 10 | 11.0 | 9000 | 1.72 |
| Example 53 | | | | | fc-6 | 20 | 19.3 | M-16 | 10 | 7.1 | | |
| Synthesis | E-33 | F-1 | 40 | 42.00 | fb-1 | 20 | 20.4 | M-4 | 10 | 8.9 | 9200 | 1.71 |
| Example 54 | | | | | fc-5 | 20 | 19.5 | M-14 | 10 | 9.2 | | |
| Synthesis | E-34 | F-1 | 40 | 41.3 | fb-3 | 20 | 19.5 | M-10 | 10 | 8.9 | 9100 | 1.56 |
| Example 55 | | | | | fc-6 | 20 | 20.7 | M-15 | 10 | 9.6 | | |

TABLE 4

| | | Monomer that gives | | | Monomer that gives | | | Monomer that gives | | | | |
|---|---|---|---|---|---|---|---|---|---|---|---|---|
| | | structural unit (I) | | | structural unit (II) | | | structural unit (III) | | | | |
| | | | | Content of | | | Content of | | | Content of | | |
| | | | Blending | structural | | Blending | structural | | Blending | structural | | |
| | Polymer | | proportion | unit | | proportion | unit | | proportion | unit | | |
| | [E] | Type | (mol %) | (mol %) | Type | (mol %) | (mol %) | Type | (mol %) | (mol %) | Mw | Mw/Mn |
| Synthesis | CE-1 | F-1 | 100 | 100 | — | — | — | — | — | — | 9100 | 1.69 |
| Example 56 | | | | | | | | | | | | |
| Synthesis | CE-2 | — | — | — | fa-1 | 35 | 36.1 | M-1 | 35 | 34.6 | 9200 | 1.68 |
| Example 57 | | | | | fb-1 | 30 | 29.3 | | | | | |
| Synthesis | CE-3 | — | — | — | fa-2 | 35 | 35.7 | M-1 | 35 | 34.6 | 8800 | 1.62 |
| Example 58 | | | | | fb-1 | 30 | 29.7 | | | | | |
| Synthesis | CE-4 | — | — | — | fa-3 | 35 | 35.9 | M-1 | 35 | 35.1 | 8700 | 1.70 |
| Example 59 | | | | | fb-1 | 30 | 29.0 | | | | | |
| Synthesis | CE-5 | — | — | — | fa-4 | 35 | 35.2 | M-1 | 35 | 35.0 | 8900 | 1.62 |
| Example 60 | | | | | fb-1 | 30 | 29.8 | | | | | |

### Preparation of Radiation-Sensitive Resin Composition

Components other than the base resin [A] and the polymer [E] used in preparation of radiation-sensitive resin compositions are shown below.

### [Radiation-Sensitive Acid Generator [B]]

B-1 to B-8: Compounds represented by formulas (B-1) to (B-8)

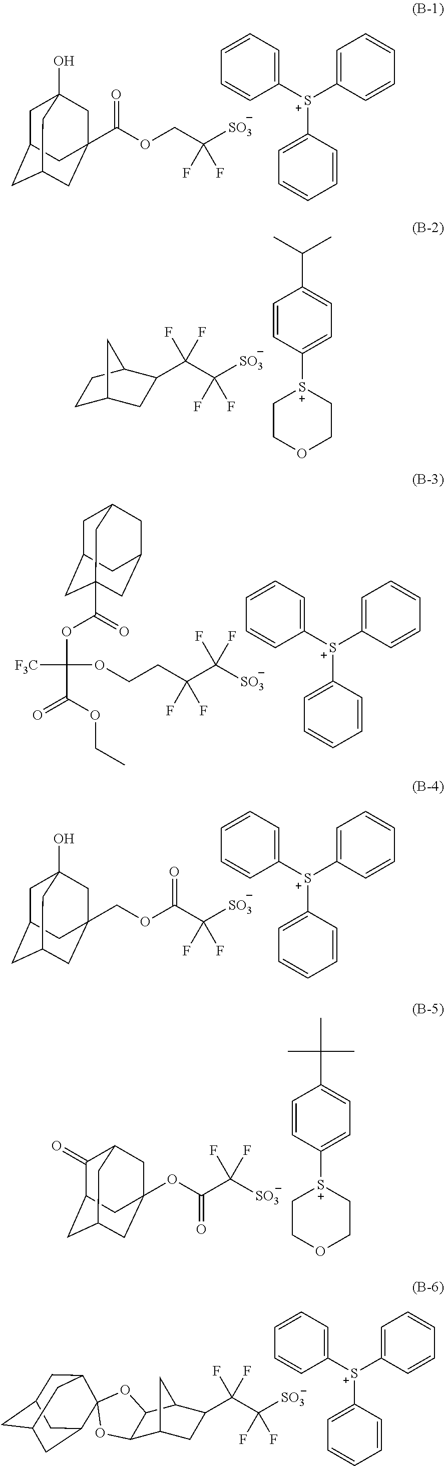

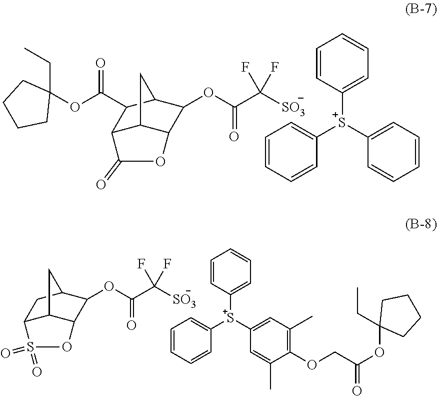

### [Acid Diffusion Controlling Agent (Onium Salt (i)) [C]]

C-1 to C-10: Compounds represented by formulas (C-1) to (C-10)
cc-1 to cc-7: Compounds represented by formulas (cc-1) to (cc-7)

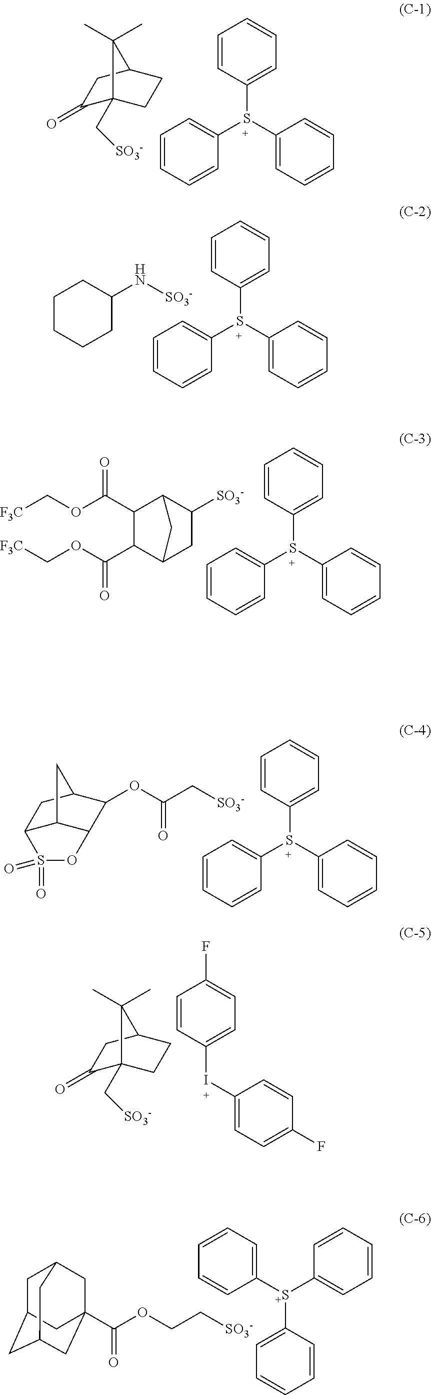

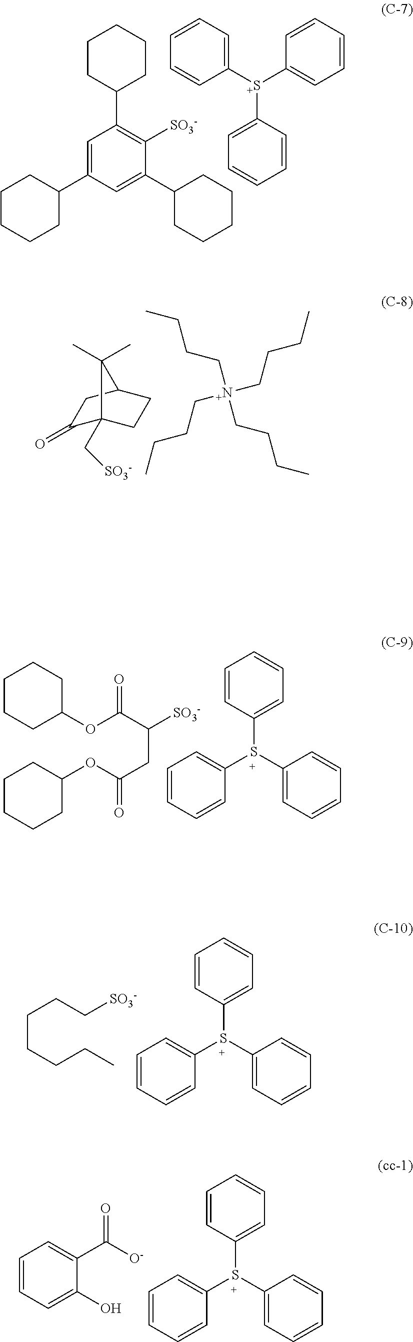

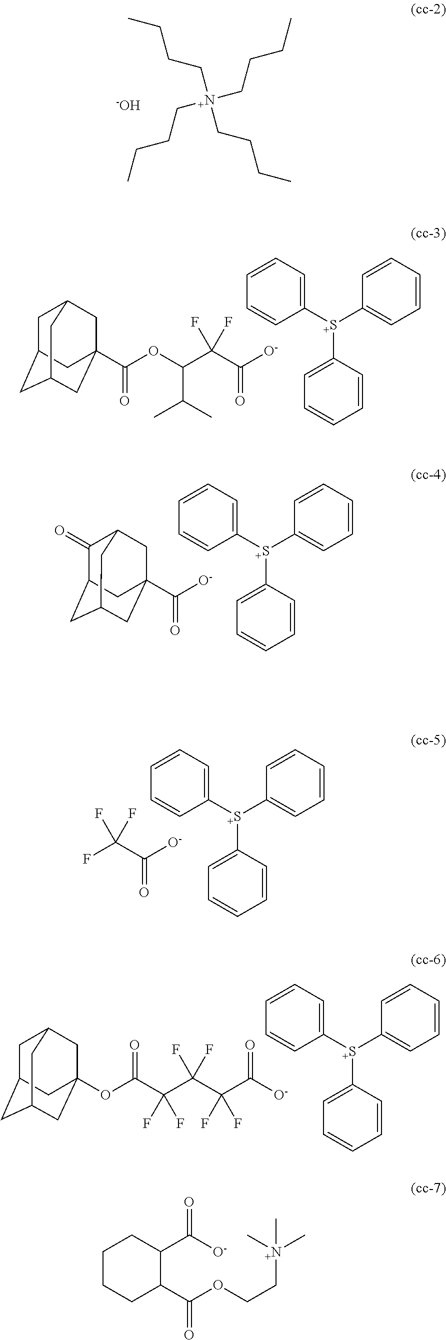

### [Solvent [D]]

D-1: Propylene glycol monomethyl ether acetate
D-2: Propylene glycol monomethyl ether
D-3: γ-Butyrolactone
D-4: Ethyl lactate

### [Preparation of Positive Radiation-Sensitive Resin Composition for ArF Exposure]

### Example 1

100 parts by mass of (A-1) as the base resin [A], 12.0 parts by mass of (B-1) as the radiation-sensitive acid generator [B], 10.0 parts by mass of (C-1) as the acid diffusion controlling agent [C], 3.0 parts by mass (solid content) of (E-1) as the polymer [E], and 3, 230 parts by mass of a mixed solvent of (D-1)/(D-2)/(D-3) as the solvent [D] were mixed, and the mixture was filtered through a membrane filter having a pore size of 0.2 μm to prepare a radiation-sensitive resin composition (J-1).

### Examples 2 to 78 and Comparative Examples 1 to 12

Radiation-sensitive resin compositions (J-2) to (J-78) and (CJ-1) to (CJ-12) were prepared in the same manner as in Example 1 except that the components of the types and contents shown in the following Tables 5-1 and 5-2 were used.

TABLE 5-1

| | | | | Radiation- | | | | | | | |
|---|---|---|---|---|---|---|---|---|---|---|---|
| | | | | sensitive acid | | Acid diffusion | | | | | |
| | | Base resin [A] | | generator [B] | | controlling agent [C] | | Polymer [E] | | Solvent [D] | |
| | Radiation- | | Content | | Content | | Content | | Content | | Content |
| | sensitive | | amount | | amount | | amount | | amount | | amount |
| | resin | | (parts by | | (parts by | | (parts by | | (parts by | | (parts by |
| | composition | Type | mass) | Type | mass) | Type | mass) | Type | mass) | Type | mass) |
| Example 1 | J-1 | A-1 | 100 | B-1 | 12.0 | C-1 | 10.0 | E-1 | 3.0 | D-1/D-2/D-3 | 2240/960/30 |
| Example 2 | J-2 | A-1 | 100 | B-1 | 12.0 | C-1 | 10.0 | E-2 | 3.0 | D-1/D-2/D-3 | 2240/960/30 |
| Example 3 | J-3 | A-1 | 100 | B-1 | 12.0 | C-1 | 10.0 | E-3 | 3.0 | D-1/D-2/D-3 | 2240/960/30 |
| Example 4 | J-4 | A-1 | 100 | B-1 | 12.0 | C-1 | 10.0 | E-4 | 3.0 | D-1/D-2/D-3 | 2240/960/30 |
| Example 5 | J-5 | A-1 | 100 | B-1 | 12.0 | C-1 | 10.0 | E-5 | 3.0 | D-1/D-2/D-3 | 2240/960/30 |
| Example 6 | J-6 | A-1 | 100 | B-1 | 12.0 | C-1 | 10.0 | E-6 | 3.0 | D-1/D-2/D-3 | 2240/960/30 |
| Example 7 | J-7 | A-1 | 100 | B-1 | 12.0 | C-1 | 10.0 | E-7 | 3.0 | D-1/D-2/D-3 | 2240/960/30 |
| Example 8 | J-8 | A-1 | 100 | B-1 | 12.0 | C-1 | 10.0 | E-8 | 3.0 | D-1/D-2/D-3 | 2240/960/30 |
| Example 9 | J-9 | A-1 | 100 | B-1 | 12.0 | C-1 | 10.0 | E-9 | 3.0 | D-1/D-2/D-3 | 2240/960/30 |
| Example 10 | J-10 | A-1 | 100 | B-1 | 12.0 | C-1 | 10.0 | E-10 | 3.0 | D-1/D-2/D-3 | 2240/960/30 |
| Example 11 | J-11 | A-1 | 100 | B-1 | 12.0 | C-1 | 10.0 | E-11 | 3.0 | D-1/D-2/D-3 | 2240/960/30 |
| Example 12 | J-12 | A-1 | 100 | B-1 | 12.0 | C-1 | 10.0 | E-12 | 3.0 | D-1/D-2/D-3 | 2240/960/30 |
| Example 13 | J-13 | A-1 | 100 | B-1 | 12.0 | C-1 | 10.0 | E-13 | 3.0 | D-1/D-2/D-3 | 2240/960/30 |
| Example 14 | J-14 | A-1 | 100 | B-1 | 12.0 | C-1 | 10.0 | E-14 | 3.0 | D-1/D-2/D-3 | 2240/960/30 |
| Example 15 | J-15 | A-1 | 100 | B-1 | 12.0 | C-1 | 10.0 | E-15 | 3.0 | D-1/D-2/D-3 | 2240/960/30 |
| Example 16 | J-16 | A-1 | 100 | B-1 | 12.0 | C-1 | 10.0 | E-16 | 3.0 | D-1/D-2/D-3 | 2240/960/30 |
| Example 17 | J-17 | A-1 | 100 | B-1 | 12.0 | C-1 | 10.0 | E-17 | 3.0 | D-1/D-2/D-3 | 2240/960/30 |
| Example 18 | J-18 | A-1 | 100 | B-1 | 12.0 | C-1 | 10.0 | E-18 | 3.0 | D-1/D-2/D-3 | 2240/960/30 |
| Example 19 | J-19 | A-1 | 100 | B-1 | 12.0 | C-1 | 10.0 | E-19 | 3.0 | D-1/D-2/D-3 | 2240/960/30 |
| Example 20 | J-20 | A-1 | 100 | B-1 | 12.0 | C-1 | 10.0 | E-20 | 3.0 | D-1/D-2/D-3 | 2240/960/30 |
| Example 21 | J-21 | A-1 | 100 | B-1 | 12.0 | C-1 | 10.0 | E-21 | 3.0 | D-1/D-2/D-3 | 2240/960/30 |
| Example 22 | J-22 | A-1 | 100 | B-1 | 12.0 | C-1 | 10.0 | E-22 | 3.0 | D-1/D-2/D-3 | 2240/960/30 |
| Example 23 | J-23 | A-1 | 100 | B-1 | 12.0 | C-1 | 10.0 | E-23 | 3.0 | D-1/D-2/D-3 | 2240/960/30 |
| Example 24 | J-24 | A-1 | 100 | B-1 | 12.0 | C-1 | 10.0 | E-24 | 3.0 | D-1/D-2/D-3 | 2240/960/30 |
| Example 25 | J-25 | A-1 | 100 | B-1 | 12.0 | C-1 | 10.0 | E-25 | 3.0 | D-1/D-2/D-3 | 2240/960/30 |
| Example 26 | J-26 | A-1 | 100 | B-1 | 12.0 | C-1 | 10.0 | E-26 | 3.0 | D-1/D-2/D-3 | 2240/960/30 |
| Example 27 | J-27 | A-1 | 100 | B-1 | 12.0 | C-1 | 10.0 | E-27 | 3.0 | D-1/D-2/D-3 | 2240/960/30 |
| Example 28 | J-28 | A-1 | 100 | B-1 | 12.0 | C-1 | 10.0 | E-28 | 3.0 | D-1/D-2/D-3 | 2240/960/30 |
| Example 29 | J-29 | A-1 | 100 | B-1 | 12.0 | C-1 | 10.0 | E-29 | 3.0 | D-1/D-2/D-3 | 2240/960/30 |
| Example 30 | J-30 | A-1 | 100 | B-1 | 12.0 | C-1 | 10.0 | E-30 | 3.0 | D-1/D-2/D-3 | 2240/960/30 |
| Example 31 | J-31 | A-1 | 100 | B-1 | 12.0 | C-1 | 10.0 | E-31 | 3.0 | D-1/D-2/D-3 | 2240/960/30 |
| Example 32 | J-32 | A-1 | 100 | B-1 | 12.0 | C-1 | 10.0 | E-32 | 3.0 | D-1/D-2/D-3 | 2240/960/30 |
| Example 33 | J-33 | A-1 | 100 | B-1 | 12.0 | C-1 | 10.0 | E-33 | 3.0 | D-1/D-2/D-3 | 2240/960/30 |
| Example 34 | J-34 | A-1 | 100 | B-1 | 12.0 | C-1 | 10.0 | E-34 | 3.0 | D-1/D-2/D-3 | 2240/960/30 |
| Example 47 | J-47 | A-2 | 100 | B-1 | 12.0 | C-1 | 10.0 | E-1 | 3.0 | D-1/D-2/D-3 | 2240/960/30 |
| Example 48 | J-48 | A-3 | 100 | B-1 | 12.0 | C-1 | 10.0 | E-1 | 3.0 | D-1/D-2/D-3 | 2240/960/30 |
| Example 49 | J-49 | A-4 | 100 | B-1 | 12.0 | C-1 | 10.0 | E-1 | 3.0 | D-1/D-2/D-3 | 2240/960/30 |
| Example 50 | J-50 | A-5 | 100 | B-1 | 12.0 | C-1 | 10.0 | E-1 | 3.0 | D-1/D-2/D-3 | 2240/960/30 |
| Example 51 | J-51 | A-6 | 100 | B-1 | 12.0 | C-1 | 10.0 | E-1 | 3.0 | D-1/D-2/D-3 | 2240/960/30 |
| Example 52 | J-52 | A-7 | 100 | B-1 | 12.0 | C-1 | 10.0 | E-1 | 3.0 | D-1/D-2/D-3 | 2240/960/30 |
| Example 53 | J-53 | A-8 | 100 | B-1 | 12.0 | C-1 | 10.0 | E-1 | 3.0 | D-1/D-2/D-3 | 2240/960/30 |
| Example 54 | J-54 | A-9 | 100 | B-1 | 12.0 | C-1 | 10.0 | E-1 | 3.0 | D-1/D-2/D-3 | 2240/960/30 |
| Example 55 | J-55 | A-10 | 100 | B-1 | 12.0 | C-1 | 10.0 | E-1 | 3.0 | D-1/D-2/D-3 | 2240/960/30 |
| Example 56 | J-56 | A-11 | 100 | B-1 | 12.0 | C-1 | 10.0 | E-1 | 3.0 | D-1/D-2/D-3 | 2240/960/30 |
| Example 57 | J-57 | A-1 | 100 | B-2 | 12.0 | C-1 | 10.0 | E-1 | 3.0 | D-1/D-2/D-3 | 2240/960/30 |
| Example 58 | J-58 | A-1 | 100 | B-3 | 12.0 | C-1 | 10.0 | E-1 | 3.0 | D-1/D-2/D-3 | 2240/960/30 |
| Example 59 | J-59 | A-1 | 100 | B-4 | 12.0 | C-1 | 10.0 | E-1 | 3.0 | D-1/D-2/D-3 | 2240/960/30 |
| Example 60 | J-60 | A-1 | 100 | B-5 | 12.0 | C-1 | 10.0 | E-1 | 3.0 | D-1/D-2/D-3 | 2240/960/30 |
| Example 61 | J-61 | A-1 | 100 | B-6 | 12.0 | C-1 | 10.0 | E-1 | 3.0 | D-1/D-2/D-3 | 2240/960/30 |
| Example 62 | J-62 | A-1 | 100 | B-7 | 12.0 | C-1 | 10.0 | E-1 | 3.0 | D-1/D-2/D-3 | 2240/960/30 |
| Example 63 | J-63 | A-1 | 100 | B-8 | 12.0 | C-1 | 10.0 | E-1 | 3.0 | D-1/D-2/D-3 | 2240/960/30 |
| Example 64 | J-64 | A-1 | 100 | B-1 | 12.0 | C-2 | 10.0 | E-1 | 3.0 | D-1/D-2/D-3 | 2240/960/30 |
| Example 65 | J-65 | A-1 | 100 | B-1 | 12.0 | C-3 | 10.0 | E-1 | 3.0 | D-1/D-2/D-3 | 2240/960/30 |
| Example 66 | J-66 | A-1 | 100 | B-1 | 12.0 | C-4 | 10.0 | E-1 | 3.0 | D-1/D-2/D-3 | 2240/960/30 |
| Example 67 | J-67 | A-1 | 100 | B-1 | 12.0 | C-5 | 10.0 | E-1 | 3.0 | D-1/D-2/D-3 | 2240/960/30 |
| Example 68 | J-68 | A-1 | 100 | B-1 | 12.0 | C-6 | 10.0 | E-1 | 3.0 | D-1/D-2/D-3 | 2240/960/30 |
| Example 69 | J-69 | A-1 | 100 | B-1 | 12.0 | C-7 | 10.0 | E-1 | 3.0 | D-1/D-2/D-3 | 2240/960/30 |
| Example 70 | J-70 | A-1 | 100 | B-1 | 12.0 | C-8 | 10.0 | E-1 | 3.0 | D-1/D-2/D-3 | 2240/960/30 |
| Example 71 | J-71 | A-1 | 100 | B-1 | 12.0 | C-9 | 10.0 | E-1 | 3.0 | D-1/D-2/D-3 | 2240/960/30 |
| Example 72 | J-72 | A-1 | 100 | B-1 | 12.0 | C-10 | 10.0 | E-1 | 3.0 | D-1/D-2/D-3 | 2240/960/30 |
| Example 73 | J-73 | A-1 | 100 | B-1 | 12.0 | C-1 | 10.0 | E-1 | 1.5 | D-1/D-2/D-3 | 2240/960/30 |
| Example 74 | J-74 | A-1 | 100 | B-1 | 12.0 | C-1 | 10.0 | E-1 | 7.0 | D-1/D-2/D-3 | 2240/960/30 |
| Example 75 | J-75 | A-1 | 100 | B-1 | 12.0 | C-1 | 10.0 | E-1 | 15.0 | D-1/D-2/D-3 | 2240/960/30 |
| Example 76 | J-76 | A-1 | 100 | B-2/B-3 | 6.0/6.0 | C-1 | 10.0 | E-1 | 3.0 | D-1/D-2/D-3 | 2240/960/30 |
| Example 77 | J-77 | A-1 | 100 | B-4/B-5 | 6.0/6.0 | C-1 | 10.0 | E-1 | 3.0 | D-1/D-2/D-3 | 2240/960/30 |
| Example 78 | J-78 | A-1 | 100 | B-6/B-8 | 6.0/6.0 | C-1 | 10.0 | E-1 | 3.0 | D-1/D-2/D-3 | 2240/960/30 |

TABLE 5-2

| | | | | Radiation- | | | | | | | |
|---|---|---|---|---|---|---|---|---|---|---|---|
| | | | | sensitive acid | | Acid diffusion | | | | | |
| | | Base resin [A] | | generator [B] | | controlling agent [C] | | Polymer [E] | | Solvent [D] | |
| | Radiation- | | Content | | Content | | Content | | Content | | Content |
| | sensitive | | amount | | amount | | amount | | amount | | amount |
| | resin | | (parts by | | (parts by | | (parts by | | (parts by | | (parts by |
| | composition | Type | mass) | Type | mass) | Type | mass) | Type | mass) | Type | mass) |
| Comparative | CJ-1 | A-1 | 100 | B-1 | 12.0 | C-1 | 10.0 | CE-1 | 3.0 | D-1/D-2/D-3 | 2240/960/30 |
| Example 1 | | | | | | | | | | | |
| Comparative | CJ-2 | A-1 | 100 | B-1 | 12.0 | C-1 | 10.0 | CE-2 | 3.0 | D-1/D-2/D-3 | 2240/960/30 |
| Example 2 | | | | | | | | | | | |
| Comparative | CJ-3 | A-1 | 100 | B-1 | 12.0 | C-1 | 10.0 | CE-3 | 3.0 | D-1/D-2/D-3 | 2240/960/30 |
| Example 3 | | | | | | | | | | | |
| Comparative | CJ-4 | A-1 | 100 | B-1 | 12.0 | C-1 | 10.0 | CE-4 | 3.0 | D-1/D-2/D-3 | 2240/960/30 |
| Example 4 | | | | | | | | | | | |
| Comparative | CJ-5 | A-1 | 100 | B-1 | 12.0 | C-1 | 10.0 | CE-5 | 3.0 | D-1/D-2/D-3 | 2240/960/30 |
| Example 5 | | | | | | | | | | | |
| Comparative | CJ-6 | A-1 | 100 | B-1 | 12.0 | cc-1 | 10.0 | E-1 | 3.0 | D-1/D-2/D-3 | 2240/960/30 |
| Example 6 | | | | | | | | | | | |
| Comparative | CJ-7 | A-1 | 100 | B-1 | 12.0 | cc-2 | 10.0 | E-1 | 3.0 | D-1/D-2/D-3 | 2240/960/30 |
| Example 7 | | | | | | | | | | | |
| Comparative | CJ-8 | A-1 | 100 | B-1 | 12.0 | cc-3 | 10.0 | E-1 | 3.0 | D-1/D-2/D-3 | 2240/960/30 |
| Example 8 | | | | | | | | | | | |
| Comparative | CJ-9 | A-1 | 100 | B-1 | 12.0 | cc-4 | 10.0 | E-1 | 3.0 | D-1/D-2/D-3 | 2240/960/30 |
| Example 9 | | | | | | | | | | | |
| Comparative | CJ-10 | A-1 | 100 | B-1 | 12.0 | cc-5 | 10.0 | E-1 | 3.0 | D-1/D-2/D-3 | 2240/960/30 |
| Example 10 | | | | | | | | | | | |
| Comparative | CJ-11 | A-1 | 100 | B-1 | 12.0 | cc-6 | 10.0 | E-1 | 3.0 | D-1/D-2/D-3 | 2240/960/30 |
| Example 11 | | | | | | | | | | | |
| Comparative | CJ-12 | A-1 | 100 | B-1 | 12.0 | cc-7 | 10.0 | E-1 | 3.0 | D-1/D-2/D-3 | 2240/960/30 |
| Example 12 | | | | | | | | | | | |

### <Formation of Resist Pattern Using Positive Radiation-Sensitive Resin Composition for ArF Exposure>

A composition for forming an underlayer antireflective film (“ARC66” manufactured by Brewer Science, Inc.) was applied onto a 12-inch silicon wafer using a spin coater (“CLEAN TRACK ACT12” manufactured by Tokyo Electron Limited), and then heated at 205° C. for 60 seconds to form an underlayer antireflective film having an average thickness of 100 nm. The positive radiation-sensitive resin composition for ArF exposure prepared above was applied onto the underlayer antireflective film using the spin coater, and subjected to PB (prebake) at 100° C. for 60 seconds. Thereafter, cooling was performed at 23° C. for 30 seconds to form a resist film having an average thickness of 90 nm. Subsequently, this resist film was exposed through a mask pattern of 50 nm line-and-space using an ArF excimer laser immersion exposure apparatus (“TWINSCAN XT-1900i” manufactured by ASML Holding N. V.) under optical conditions of a numeral aperture (NA) of 1.35 and dipole illumination (σ=0.9/0.7). After the exposure, PEB (post exposure bake) was performed at 100° C. for 60 seconds. Thereafter, the resist film was subjected to alkaline development using a 2.38 mass % aqueous TMAH solution as an alkaline developer. After the development, the resist film was washed with water and further dried to form a positive resist pattern (50 nm line-and-space pattern).

### Evaluation

The resist patterns formed using the positive radiation-sensitive resin compositions for ArF exposure were evaluated on sensitivity, LWR performance, storage stability, and number of development defects according to the following methods. The receding contact angle of the resist films before ArF exposure was evaluated according to the following method. Table 6 below shows the results. A scanning electron microscope (“CG-5000” of Hitachi High-Tech Corporation) was used for measuring the length of the resist pattern.

### [Sensitivity]

An exposure dose at which a 50 nm line-and-space pattern was formed in formation of a resist pattern using the positive radiation-sensitive resin composition for ArF exposure was defined as an optimum exposure dose, and this optimum exposure dose was defined as sensitivity (mJ/cm²). A case where the sensitivity was 35 mJ/cm² or less was evaluated as “good”, and a case where the sensitivity exceeded 35 mJ/cm² was evaluated as “poor”.

### [LWR Performance]

A resist pattern was formed by irradiation with the optimum exposure dose determined in the evaluation of the sensitivity, with the size of a mask adjusted so as to form a 50-nm line-and-space pattern. The formed resist pattern was observed from above the pattern using the scanning electron microscope. The variation in line width was measured at 500 points in total, the value of 30 was obtained from the distribution of the measured values, and the value of 30 was defined as LWR (nm). A smaller value of LWR indicates smaller roughness of the line and better performance. The LWR performance was evaluated as “good” when the LWR was 3.0 nm or less, and was evaluated as “poor” when the LWR exceeded 3.0 nm.

[Receding Contact Angle after PB]

The receding contact angle of the resist films before ArF exposure in the method for forming a resist pattern was measured by the following procedure in the environment at a room temperature of 23° C., a relative humidity of 40%, and normal pressure, using DSA-10 of KRUSS Optronic GmbH.

A 25-μL water drop was formed on the resist film by discharging water through a needle of DSA-10, and then suctioned by the needle at a rate of 10 μL/min for 90 seconds, and the contact angle was measured every minute (total 90 times). The average value of total 20 contact angles measured from the stabilization of the contact angle in the measurement was calculated and defined as a receding contact angle (°) after PB. When being 70° or more, the receding contact angle after PB was evaluated as “good”, and when less than 70°, evaluated as “poor”.

### [Storage Stability]

The positive radiation-sensitive resin compositions for ArF exposure were stored at 40° C. for 1 month, and then the receding contact angle after PB was evaluated in the same manner as in the method described above. The change rate of the receding contact angle after PB between before and after the storage was obtained by a formula below. When being 0.5% or less, the change rate of the receding contact angle was evaluated as “A” (very good), when more than 0.5% and 1.0% or less, evaluated as “B” (good), and when more than 1.0%, evaluated as “C” (poor).

$$Change rate of receding contact angle ( \% ) = \{ ( {θ}_{0} - {θ}_{1} ) / {θ}_{0} \} \times 100$$

(θ₀ is a receding contact angle before the storage, and θ₁ is a receding contact angle after the 1-month storage.)

### [Number of Development Defects]

The resist films were exposed with the optimum exposure dose to form a 50-nm line-and-space pattern and used as wafers for defect inspection. The number of defects on this wafer for defect inspection was measured using a defect inspection device (“KLA 2810” of KLA-Tencor Corporation). The defects having a diameter of 50 μm or less were determined to be derived from the resist film, and the number of the defects were calculated. When the number of defects determined to be derived from the resist film was 50 or less, the number of development defects was evaluated as “good”, and when more than 50, evaluated as “poor”.

TABLE 6-1

| | | | | Receding contact | | Number of |
|---|---|---|---|---|---|---|
| | Radiation-sensitive | Sensitivity | | angle after | Storage | development |
| | resin composition | (mJ/cm²) | LWR (nm) | PB (°) | stability | defects |
| Example 1 | J-1 | 28 | 2.5 | 77 | A | 5 |
| Example 2 | J-2 | 28 | 2.7 | 80 | A | 4 |
| Example 3 | J-3 | 27 | 2.6 | 79 | A | 7 |
| Example 4 | J-4 | 28 | 2.6 | 81 | A | 8 |
| Example 5 | J-5 | 29 | 2.5 | 72 | A | 5 |
| Example 6 | J-6 | 28 | 2.4 | 75 | A | 3 |
| Example 7 | J-7 | 28 | 2.5 | 78 | A | 6 |
| Example 8 | J-8 | 28 | 2.6 | 81 | A | 8 |
| Example 9 | J-9 | 28 | 2.6 | 79 | A | 5 |
| Example 10 | J-10 | 27 | 2.7 | 79 | A | 4 |
| Example 11 | J-11 | 29 | 2.5 | 74 | A | 3 |
| Example 12 | J-12 | 28 | 2.5 | 75 | A | 2 |
| Example 13 | J-13 | 28 | 2.4 | 72 | A | 1 |
| Example 14 | J-14 | 28 | 2.5 | 78 | A | 3 |
| Example 15 | J-15 | 28 | 2.6 | 79 | A | 8 |
| Example 16 | J-16 | 29 | 2.6 | 75 | A | 1 |
| Example 17 | J-17 | 28 | 2.5 | 79 | A | 2 |
| Example 18 | J-18 | 27 | 2.5 | 75 | A | 5 |
| Example 19 | J-19 | 28 | 2.6 | 79 | A | 1 |
| Example 20 | J-20 | 27 | 2.6 | 78 | A | 6 |
| Example 21 | J-21 | 28 | 2.7 | 77 | A | 1 |
| Example 22 | J-22 | 28 | 2.4 | 80 | A | 6 |
| Example 23 | J-23 | 27 | 2.6 | 72 | A | 7 |
| Example 24 | J-24 | 29 | 2.6 | 73 | A | 8 |
| Example 25 | J-25 | 29 | 2.5 | 74 | A | 6 |
| Example 26 | J-26 | 28 | 2.5 | 80 | A | 8 |
| Example 27 | J-27 | 27 | 2.6 | 76 | A | 2 |
| Example 28 | J-28 | 28 | 2.6 | 82 | A | 4 |
| Example 29 | J-29 | 29 | 2.7 | 82 | A | 6 |
| Example 30 | J-30 | 28 | 2.5 | 80 | A | 2 |
| Example 31 | J-31 | 27 | 2.5 | 77 | A | 8 |
| Example 32 | J-32 | 28 | 2.5 | 81 | A | 6 |
| Example 33 | J-33 | 28 | 2.4 | 82 | A | 5 |
| Example 34 | J-34 | 29 | 2.5 | 80 | A | 7 |
| Example 47 | J-47 | 28 | 2.6 | 77 | A | 7 |
| Example 48 | J-48 | 27 | 2.6 | 78 | A | 4 |
| Example 49 | J-49 | 28 | 2.7 | 77 | A | 6 |
| Example 50 | J-50 | 27 | 2.4 | 76 | A | 7 |
| Example 51 | J-51 | 28 | 2.5 | 77 | A | 8 |
| Example 52 | J-52 | 28 | 2.6 | 78 | A | 4 |
| Example 53 | J-53 | 27 | 2.6 | 78 | A | 3 |
| Example 54 | J-54 | 28 | 2.5 | 77 | A | 7 |
| Example 55 | J-55 | 28 | 2.5 | 78 | A | 4 |
| Example 56 | J-56 | 28 | 2.6 | 77 | A | 7 |
| Example 57 | J-57 | 26 | 2.7 | 77 | A | 3 |
| Example 58 | J-58 | 25 | 2.8 | 77 | A | 6 |
| Example 59 | J-59 | 28 | 2.6 | 77 | A | 7 |
| Example 60 | J-60 | 29 | 2.5 | 78 | A | 8 |
| Example 61 | J-61 | 28 | 2.5 | 77 | A | 2 |
| Example 62 | J-62 | 26 | 2.6 | 77 | A | 6 |
| Example 63 | J-63 | 29 | 2.5 | 76 | A | 7 |
| Example 64 | J-64 | 29 | 2.7 | 77 | A | 2 |
| Example 65 | J-65 | 25 | 2.6 | 77 | A | 4 |
| Example 66 | J-66 | 26 | 2.6 | 78 | A | 1 |
| Example 67 | J-67 | 28 | 2.8 | 78 | A | 5 |
| Example 68 | J-68 | 25 | 2.3 | 78 | A | 6 |
| Example 69 | J-69 | 27 | 2.5 | 76 | A | 8 |
| Example 70 | J-70 | 28 | 2.6 | 76 | A | 2 |
| Example 71 | J-71 | 26 | 2.8 | 78 | A | 5 |
| Example 72 | J-72 | 32 | 2.8 | 77 | A | 25 |
| Example 73 | J-73 | 28 | 2.5 | 73 | A | 8 |
| Example 74 | J-74 | 28 | 2.5 | 77 | A | 11 |
| Example 75 | J-75 | 28 | 2.7 | 72 | A | 21 |
| Example 76 | J-76 | 29 | 2.5 | 76 | A | 5 |
| Example 77 | J-77 | 29 | 2.7 | 77 | A | 6 |
| Example 78 | J-78 | 25 | 2.6 | 77 | A | 3 |

TABLE 6-2

| | | | | Receding contact | | Number of |
|---|---|---|---|---|---|---|
| | Radiation-sensitive | Sensitivity | LWR | angle after | Storage | development |
| | resin composition | (mJ/cm²) | (nm) | PB (°) | stability | defects |
| Comparative Example 1 | CJ-1 | 32 | 3.3 | 62 | A | 128 |
| Comparative Example 2 | CJ-2 | 31 | 3.5 | 65 | C | 231 |
| Comparative Example 3 | CJ-3 | 30 | 3.2 | 62 | B | 285 |
| Comparative Example 4 | CJ-4 | 31 | 3.5 | 68 | B | 264 |
| Comparative Example 5 | CJ-5 | 37 | 3.4 | 62 | A | 375 |
| Comparative Example 6 | CJ-6 | 38 | 3.4 | 66 | C | 210 |
| Comparative Example 7 | CJ-7 | 40 | 3.5 | 58 | C | 423 |
| Comparative Example 8 | CJ-8 | 41 | 3.4 | 60 | C | 395 |
| Comparative Example 9 | CJ-9 | 40 | 3.7 | 62 | C | 259 |
| Comparative Example 10 | CJ-10 | 39 | 3.4 | 67 | C | 294 |
| Comparative Example 11 | CJ-11 | 41 | 3.4 | 61 | C | 301 |
| Comparative Example 12 | CJ-12 | 40 | 3.3 | 68 | C | 354 |

As is apparent from the results in Tables 6-1 and 6-2, the radiation-sensitive resin compositions of the examples were good in sensitivity, LWR performance, receding contact angle after PB, storage stability, and number of development defects when used for ArF exposure, whereas the comparative examples were poorer in the characteristics than the examples. Therefore, when the radiation-sensitive resin compositions of the examples are used for ArF exposure, a resist pattern having good LWR performance, water repellency, storage stability, and defection performance can be formed with high sensitivity.

### [Preparation of Positive Radiation-Sensitive Resin Composition for Extreme Ultraviolet (EUV) Exposure]

### Example 79

One hundred parts by mass of (A-12) as the base resin [A], 15.0 parts by mass of (B-5) as the radiation-sensitive acid generator [B], 12.0 parts by mass of (C-5) as the acid diffusion controlling agent [C], 3.0 parts by mass (solid content) of (E-1) as the polymer [E], and 6, 110 parts by mass of a mixed solvent of (D-1)/(D-4) as the solvent [D] were mixed and filtered through a membrane filter having a pore size of 0.2 μm to prepare a radiation-sensitive resin composition (J-79).

### Examples 80 to 92 and Comparative Examples 13 to 18

Radiation-sensitive resin compositions (J-80) to (J-92) and (CJ-13) to (CJ-18) were prepared in the same manner as in Example 79 except that the types of components and the content amounts shown in Table 7 below were used.

TABLE 7

| | | | | Radiation- | | | | | | | |
|---|---|---|---|---|---|---|---|---|---|---|---|
| | | | | sensitive acid | | Acid diffusion | | | | | |
| | | Base resin [A] | | generator [B] | | controlling agent [C] | | Polymer [E] | | Solvent [D] | |
| | Radiation- | | Content | | Content | | Content | | Content | | Content |
| | sensitive | | amount | | amount | | amount | | amount | | amount |
| | resin | | (parts by | | (parts by | | (parts by | | (parts by | | (parts by |
| | composition | Type | mass) | Type | mass) | Type | mass) | Type | mass) | Type | mass) |
| Example 79 | J-79 | A-12 | 100 | B-5 | 15.0 | C-5 | 12.0 | E-1 | 3.0 | D-1/D-4 | 4280/1830 |
| Example 80 | J-80 | A-12 | 100 | B-5 | 15.0 | C-5 | 12.0 | E-31 | 3.0 | D-1/D-4 | 4280/1830 |
| Example 81 | J-81 | A-12 | 100 | B-5 | 15.0 | C-5 | 12.0 | E-32 | 3.0 | D-1/D-4 | 4280/1830 |
| Example 82 | J-82 | A-12 | 100 | B-5 | 15.0 | C-5 | 12.0 | E-33 | 3.0 | D-1/D-4 | 4280/1830 |
| Example 83 | J-83 | A-12 | 100 | B-5 | 15.0 | C-5 | 12.0 | E-34 | 3.0 | D-1/D-4 | 4280/1830 |
| Example 84 | J-84 | A-12 | 100 | B-5 | 15.0 | C-2 | 12.0 | E-1 | 3.0 | D-1/D-4 | 4280/1830 |
| Example 85 | J-85 | A-12 | 100 | B-5 | 15.0 | C-4 | 12.0 | E-1 | 3.0 | D-1/D-4 | 4280/1830 |
| Example 86 | J-86 | A-12 | 100 | B-5 | 15.0 | C-8 | 12.0 | E-1 | 3.0 | D-1/D-4 | 4280/1830 |
| Example 87 | J-87 | A-13 | 100 | B-5 | 15.0 | C-5 | 12.0 | E-1 | 3.0 | D-1/D-4 | 4280/1830 |
| Example 88 | J-88 | A-14 | 100 | B-5 | 15.0 | C-5 | 12.0 | E-1 | 3.0 | D-1/D-4 | 4280/1830 |
| Example 89 | J-89 | A-15 | 100 | B-5 | 15.0 | C-5 | 12.0 | E-1 | 3.0 | D-1/D-4 | 4280/1830 |
| Example 90 | J-90 | A-12 | 100 | B-3 | 15.0 | C-5 | 12.0 | E-1 | 3.0 | D-1/D-4 | 4280/1830 |
| Example 91 | J-91 | A-12 | 100 | B-7 | 15.0 | C-5 | 12.0 | E-1 | 3.0 | D-1/D-4 | 4280/1830 |
| Example 92 | J-92 | A-12 | 100 | B-8 | 15.0 | C-5 | 12.0 | E-1 | 3.0 | D-1/D-4 | 4280/1830 |
| Comparative | CJ-13 | A-12 | 100 | B-5 | 15.0 | C-5 | 12.0 | CE-1 | 3.0 | D-1/D-4 | 4280/1830 |
| Example 13 | | | | | | | | | | | |
| Comparative | CJ-14 | A-12 | 100 | B-5 | 15.0 | C-5 | 12.0 | CE-2 | 3.0 | D-1/D-4 | 4280/1830 |
| Example 14 | | | | | | | | | | | |
| Comparative | CJ-15 | A-12 | 100 | B-5 | 15.0 | cc-2 | 12.0 | E-1 | 3.0 | D-1/D-4 | 4280/1830 |
| Example 15 | | | | | | | | | | | |
| Comparative | CJ-16 | A-12 | 100 | B-5 | 15.0 | cc-4 | 12.0 | E-1 | 3.0 | D-1/D-4 | 4280/1830 |
| Example 16 | | | | | | | | | | | |
| Comparative | CJ-17 | A-12 | 100 | B-5 | 15.0 | cc-5 | 12.0 | E-1 | 3.0 | D-1/D-4 | 4280/1830 |
| Example 17 | | | | | | | | | | | |
| Comparative | CJ-18 | A-12 | 100 | B-5 | 15.0 | cc-6 | 12.0 | E-1 | 3.0 | D-1/D-4 | 4280/1830 |
| Example 18 | | | | | | | | | | | |

### <Formation of Resist Pattern Using Positive Radiation-Sensitive Resin Composition for EUV Exposure>

A composition for forming an underlayer antireflective film (“ARC66” manufactured by Brewer Science, Inc.) was applied onto a 12-inch silicon wafer using a spin coater (“CLEAN TRACK ACT12” manufactured by Tokyo Electron Limited), and then heated at 205° C. for 60 seconds to form an underlayer antireflective film having an average thickness of 105 nm. The positive radiation-sensitive resin composition for EUV exposure prepared above was applied onto the underlayer antireflective film using the spin coater, and subjected to PB at 130° C. for 60 seconds. Thereafter, cooling was performed at 23° C. for 30 seconds to form a resist film having an average thickness of 55 nm. Subsequently, this resist film was exposed with an EUV exposure apparatus (“NXE3300” manufactured by ASML Holding N.V.) with an NA of 0.33 under an illumination condition of conventional illumination (s=0.89), and with a mask of imecDEFECT32FFR02. After the exposure, PEB was performed at 120° C. for 60 seconds. Thereafter, the resist film was subjected to alkaline development using a 2.38 mass % aqueous TMAH solution as an alkaline developer, and after the development, the resist film was washed with water and further dried to form a positive resist pattern (32 nm line-and-space pattern).

### Evaluation

The resist patterns formed using the positive radiation-sensitive resin compositions for EUV exposure were evaluated on sensitivity, LWR performance, storage stability, and number of development defects according to the following methods. The receding contact angle of the resist films before EUV exposure was evaluated according to the following method. Table 8 below shows the results. A scanning electron microscope (“CG-5000” of Hitachi High-Tech Corporation) was used for measuring the length of the resist pattern.

### [Sensitivity]

In formation of the resist pattern using the positive radiation-sensitive resin composition for EUV exposure, an exposure dose at which a 32 nm line-and-space pattern was formed was defined as an optimum exposure dose, and this optimum exposure dose was defined as sensitivity (mJ/cm²). A case where the sensitivity was 30 mJ/cm² or less was evaluated as “good”, and a case where the sensitivity exceeded 30 mJ/cm² was evaluated as “poor”.

### [LWR Performance]

A resist pattern was formed with the mask size adjusted so as to form a 32 nm line-and-space pattern by irradiation with the optimum exposure dose obtained in the evaluation of the sensitivity. The formed resist pattern was observed from above the pattern using the scanning electron microscope. The variation in line width was measured at 500 points in total, the value of 30 was obtained from the distribution of the measured values, and the value of 30 was defined as LWR (nm). A smaller value of LWR indicates smaller roughness of the line and better performance. A case where the LWR performance was 3.0 nm or less was evaluated as “good”, and a case where the LWR performance exceeded 3.0 nm was evaluated as “poor”.

[Receding Contact Angle after PB]

The receding contact angle of the resist films before EUV exposure in the method for forming a resist pattern was measured by the following procedure in the environment at a room temperature of 23° C., a relative humidity of 40%, and normal pressure, using DSA-10 of KRUSS Optronic GmbH.

A 25-μL water drop was formed on the resist film by discharging water through a needle of DSA-10, and then suctioned by the needle at a rate of 10 μL/min for 90 seconds, and the contact angle was measured every minute (total 90 times). The average value of total 20 contact angles measured from the stabilization of the contact angle in the measurement was calculated and defined as a receding contact angle (°) after PB. When being 70° or more, the receding contact angle after PB was evaluated as “good”, and when less than 70°, evaluated as “poor”.

### [Storage Stability]

The positive radiation-sensitive resin compositions for EUV exposure were stored at 40° C. for 1 month, and then the receding contact angle after PB was evaluated in the same manner as in the method described above. The change rate of the receding contact angle after PB between before and after the storage was obtained by a formula below. When being 0.5% or less, the change rate of the receding contact angle was evaluated as “A” (very good), when more than 0.5% and 1.0% or less, evaluated as “B” (good), and when more than 1.0%, evaluated as “C” (poor).

$$Change rate of receding contact angle ( \% ) = \{ ( {θ}_{0} - {θ}_{1} ) / {θ}_{0} \} \times 100$$

(θ₀ is a receding contact angle before the storage, and θ₁ is a receding contact angle after the 1-month storage.)

### [Number of Development Defects]

The resist films were exposed with the optimum exposure dose to form a 32-nm line-and-space pattern and used as wafers for defect inspection. The number of defects on this wafer for defect inspection was measured using a defect inspection device (“KLA 2810” of KLA-Tencor Corporation). The defects having a diameter of 50 μm or less were determined to be derived from the resist film, and the number of the defects were calculated. When the number of defects determined to be derived from the resist film was 50 or less, the number of development defects was evaluated as “good”, and when more than 50, evaluated as “poor”.

TABLE 8

| | | | | Receding contact | | Number of |
|---|---|---|---|---|---|---|
| | Radiation-sensitive | Sensitivity | LWR | angle after | Storage | development |
| | resin composition | (mJ/cm²) | (nm) | PB (°) | stability | defects |
| Example 79 | J-79 | 21 | 2.6 | 75 | A | 4 |
| Example 80 | J-80 | 20 | 2.7 | 77 | A | 2 |
| Example 81 | J-81 | 22 | 2.2 | 76 | A | 5 |
| Example 82 | J-82 | 22 | 2.8 | 78 | A | 6 |
| Example 83 | J-83 | 23 | 2.5 | 77 | A | 3 |
| Example 84 | J-84 | 24 | 2.2 | 75 | A | 6 |
| Example 85 | J-85 | 22 | 2.6 | 75 | A | 2 |
| Example 86 | J-86 | 22 | 2.3 | 76 | A | 5 |
| Example 87 | J-87 | 21 | 2.5 | 75 | A | 6 |
| Example 88 | J-88 | 21 | 2.4 | 74 | A | 3 |
| Example 89 | J-89 | 20 | 2.6 | 75 | A | 2 |
| Example 90 | J-90 | 22 | 2.8 | 75 | A | 6 |
| Example 91 | J-91 | 22 | 2.7 | 75 | A | 3 |
| Example 92 | J-92 | 21 | 2.5 | 76 | A | 8 |
| Comparative Example 13 | CJ-13 | 22 | 3.3 | 66 | A | 421 |
| Comparative Example 14 | CJ-14 | 23 | 3.1 | 62 | C | 293 |
| Comparative Example 15 | CJ-15 | 32 | 3.1 | 59 | C | 533 |
| Comparative Example 16 | CJ-16 | 33 | 3.4 | 62 | C | 378 |
| Comparative Example 17 | CJ-17 | 34 | 3.2 | 61 | C | 320 |
| Comparative Example 18 | CJ-18 | 33 | 3.3 | 63 | C | 398 |

As is apparent from the results in Table 8, the radiation-sensitive resin compositions of the examples were good in sensitivity, LWR performance, receding contact angle after PB, storage stability and number of development defects when used for EUV exposure, whereas the comparative examples were poorer in the characteristics than the examples.

Therefore, when the radiation-sensitive resin compositions of the examples are used for EUV exposure, a resist pattern having good LWR performance, water repellency, storage stability, and defection performance can be formed with high sensitivity.

[Preparation of Negative Radiation-Sensitive Resin Composition for ArF exposure, formation of resist pattern using this composition, and evaluation]

### Example 93

One hundred parts by mass of (A-10) as the base resin [A], 12.0 parts by mass of (B-4) as the radiation-sensitive acid generator [B], 12.0 parts by mass of (C-3) as the acid diffusion controlling agent [C], 4.0 parts by mass (solid content) of (E-32) as the polymer [E], and 3,230 parts by mass of a mixed solvent of (D-1)/(D-2)/(D-3) (mass ratio 2240 parts/960 parts/30 parts) as the solvent [D] were mixed and filtered through a membrane filter having a pore size of 0.2 μm to prepare a radiation-sensitive resin composition (J-93).

A composition for forming an underlayer antireflective film (“ARC66” manufactured by Brewer Science, Inc.) was applied onto a 12-inch silicon wafer using a spin coater (“CLEAN TRACK ACT12” manufactured by Tokyo Electron Limited), and then heated at 205° C. for 60 seconds to form an underlayer antireflective film having an average thickness of 100 nm. The negative radiation-sensitive resin composition for ArF exposure (J-93) prepared above was applied onto the underlayer antireflective film using the spin coater, and subjected to PB (prebake) at 100° C. for 60 seconds. Thereafter, cooling was performed at 23° C. for 30 seconds to form a resist film having an average thickness of 90 nm. Subsequently, this resist film was exposed through a mask pattern of 40 nm hole and 105 nm pitch using an ArF excimer laser immersion exposure apparatus (“TWINSCAN XT-1900i” manufactured by ASML Holding N. V.) under optical conditions of a numeral aperture (NA) of 1.35 and annular illumination (σ=0.8/0.6). After the exposure, PEB (post exposure bake) was performed at 100° C. for 60 seconds. Thereafter, the resist film was developed with n-butyl acetate as an organic solvent developer, and dried to form a negative resist pattern (40 nm hole, 105 nm pitch).

### Evaluation

The resist patterns formed using the negative radiation-sensitive resin compositions for ArF exposure were evaluated on sensitivity, receding contact angle after PB, number of development defects, and storage stability according to the following methods. A scanning electron microscope (“CG-5000” of Hitachi High-Tech Corporation) was used for measuring the length of the resist pattern.

The resist pattern and resist film before ArF exposure formed using the negative radiation-sensitive resin composition for ArF exposure were evaluated in the same manner as in the evaluation of the resist patterns formed using the positive radiation-sensitive resin compositions for ArF exposure. As a result, when forming a negative resist pattern by ArF exposure, the radiation-sensitive resin composition of Example 93 gave high sensitivity and good receding contact angle after PB, number of development defects, and storage stability.

[Preparation of Negative Radiation-Sensitive Resin Composition for EUV exposure, formation of resist pattern using this composition, and evaluation]

### Example 94

One hundred parts by mass of (A-14) as the base resin [A], 20.0 parts by mass of (B-8) as the radiation-sensitive acid generator [B], 18.0 parts by mass of (C-2) as the acid diffusion controlling agent [C], 3.0 parts by mass (solid content) of (E-32) as the polymer [E], and 6,110 parts by mass of a mixed solvent of (D-1)/(D-4) (mass ratio 4280 parts/1830 parts) as the solvent [D] were mixed and filtered through a membrane filter having a pore size of 0.2 μm to prepare a radiation-sensitive resin composition (J-94).

A composition for forming an underlayer antireflective film (“ARC66” manufactured by Brewer Science, Inc.) was applied onto a 12-inch silicon wafer using a spin coater (“CLEAN TRACK ACT12” manufactured by Tokyo Electron Limited), and then heated at 205° C. for 60 seconds to form an underlayer antireflective film having an average thickness of 105 nm. The negative radiation-sensitive resin composition for EUV exposure (J-94) prepared above was applied onto the underlayer antireflective film using the spin coater, and subjected to PB at 130° C. for 60 seconds. Thereafter, cooling was performed at 23° C. for 30 seconds to form a resist film having an average thickness of 55 nm. Subsequently, this resist film was exposed with an EUV exposure apparatus (“NXE3300” manufactured by ASML Holding N.V.) with an NA of 0.33 under an illumination condition of conventional illumination (s=0.89), and with a mask of imecDEFECT32FFR02. After the exposure, PEB was performed at 120° C. for 60 seconds. Thereafter, the resist film was developed with n-butyl acetate as an organic solvent developer, and dried to form a negative resist pattern (40 nm hole, 105 nm pitch).

The resist pattern formed using the negative radiation-sensitive resin composition for EUV exposure was evaluated in the same manner as in the evaluation of the resist pattern formed using the negative radiation-sensitive resin composition for ArF exposure. As a result, when forming a negative resist pattern by EUV exposure, the radiation-sensitive resin composition of Example 94 gave high sensitivity and good receding contact angle after PB, number of development defects, and storage stability.

According to the radiation-sensitive resin composition and the method for forming a resist pattern of the present disclosure, the composition has excellent storage stability and good sensitivity to exposure light, and is capable of forming a resist pattern having LWR performance, water repellency, and less defects. Therefore, these composition and method can suitably be used for a machining process and the like of a semiconductor device that is expected to be further miniaturized in the future.

Obviously, numerous modifications and variations of the present invention(s) are possible in light of the above teachings. It is therefore to be understood that within the scope of the appended claims, the invention(s) may be practiced otherwise than as specifically described herein.

What is claimed is:

1. A radiation-sensitive resin composition comprising:
a polymer comprising a structural unit (I) represented by formula (1) and a structural unit different from the structural unit (I);
an onium salt represented by formula (i); and
a solvent, 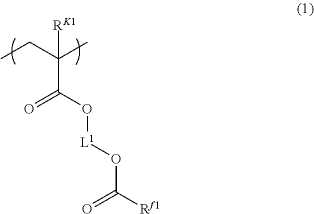
wherein, in formula (1),
R^{K1} is a hydrogen atom, a fluorine atom, a methyl group, or a trifluoromethyl group,
L¹ is an alkanediyl group having 1 to 5 carbon atoms, and
R^{f1} is a fluorinated hydrocarbon group having 2 to 10 carbon atoms and 5 to 7 fluorine atoms; 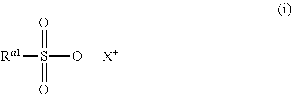
wherein, in formula (i),
R^{a1} is a substituted or unsubstituted monovalent organic group having 1 to 40 carbon atoms with no fluorine atom or fluorinated hydrocarbon group attached to an atom adjacent to the sulfur atom, and
X⁺ is a monovalent onium cation.

2. The radiation-sensitive resin composition according to claim 1, wherein in formula (i), R^{a1} comprises a cyclic structure.

3. The radiation-sensitive resin composition according to claim 1, wherein a content of the onium salt is 0.1% by mass or more and 30% by mass or less relative to 100% by mass of a total mass of components other than the solvent in the radiation-sensitive resin composition.

4. The radiation-sensitive resin composition according to claim 1, wherein in formula (1), R^{f1} is a fluorinated linear hydrocarbon group having 2 to 4 carbon atoms and 5 fluorine atoms.

5. The radiation-sensitive resin composition according to claim 1, wherein in formula (1), L¹ is a methanediyl group or an ethanediyl group.

6. The radiation-sensitive resin composition according to claim 1, wherein a content of the structural unit (I) in the polymer is 5.0 mol % or more and 95.0 mol % or less relative to 100 mol % of all structural units constituting the polymer.

7. The radiation-sensitive resin composition according to claim 1, wherein the polymer further comprises, as a structural unit different from the structural unit (I), a structural unit (II) represented by a formula (2) but excluding a structure corresponding to the structural unit (I), 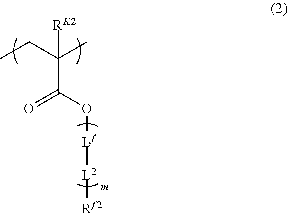
wherein, in formula (2)
R^{K2} is a hydrogen atom, a fluorine atom, a methyl group, or a trifluoromethyl group,
L^{f} is a fluorine-substituted or unsubstituted divalent organic group having 1 to 20 carbon atoms,
L² is *—COO— or *—OCO—, * is a bond on an L^{f} side,
m is an integer of 0 to 2, when a plurality of L^{f}s and L²s are present, the plurality of L^{f}s are identical or different from each other, and the plurality of Les are identical or different from each other,
R^{f2} is a fluorine-substituted or unsubstituted monovalent organic group having 1 to 20 carbon atoms, and
at least one of L^{f} and R^{f2} comprises one or more fluorine atom in total.

8. The radiation-sensitive resin composition according to claim 1, wherein the polymer further comprises, as a structural unit different from the structural unit (I), a structural unit (III) represented by a formula (3), 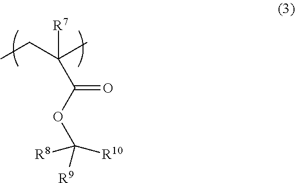
wherein
R⁷ is a hydrogen atom, a fluorine atom, a methyl group, or a trifluoromethyl group,
R⁸ is a monovalent hydrocarbon group having 1 to 20 carbon atoms, and
R⁹ and R¹⁰ each independently represent a monovalent chain hydrocarbon group having 1 to 10 carbon atoms or a monovalent alicyclic hydrocarbon group having 3 to 20 carbon atoms, or R⁹ and R¹⁰ taken together represent a divalent alicyclic group having 3 to 20 carbon atoms together with the carbon atom to which R⁹ and R¹⁰ are bonded.

9. The radiation-sensitive resin composition according to claim 1, further comprising a resin that contains a structural unit having an acid-dissociable group, and that has a lower mass content of fluorine atoms than a mass content of fluorine atoms of the polymer.

10. The radiation-sensitive resin composition according to claim 1, further comprising a radiation-sensitive acid generator.

11. A method for forming a pattern, the method comprising:
directly or indirectly applying the radiation-sensitive resin composition according to claim 1 to a substrate to form a resist film;
exposing the resist film; and
developing the exposed resist film with a developer.

12. The method according to claim 11, wherein the developing is performed using an alkaline aqueous solution.
